(19) United States
(12) Patent Application Publication
(10) Pub. No.: US 2023/0201118 A1
(43) Pub. Date: Jun. 29, 2023

(54) PRODRUG COPOLYMERS AND POLYMERIC MICELLES THEREOF FOR THE DELIVERY OF SHORT-CHAIN FATTY ACIDS, THE PROMOTION OF GUT HEALTH, AND THE TREATMENT OF IMMUNE AND/OR INFLAMMATORY CONDITIONS AND FOOD ALLERGY

(71) Applicant: The University of Chicago, Chicago, IL (US)

(72) Inventors: Jeffrey Hubbell, Chicago, IL (US); Ruyi Wang, Harrison, NJ (US); Shijie Cao, Chicago, IL (US); Cathryn R. Nagler, Chicago, IL (US); D. Scott Wilson, Baltimore, MD (US); Mohamed H. Bashir, Oak Lawn, IL (US)

(21) Appl. No.: 18/052,449

(22) Filed: Nov. 3, 2022

Related U.S. Application Data
(60) Provisional application No. 63/329,913, filed on Apr. 12, 2022.
(60) Provisional application No. 63/275,260, filed on Nov. 3, 2021.

(51) Int. Cl. A61K 9/107; A61K 47/32

(52) U.S. Cl. CPC A61K 9/1075; A61K 47/32

(57) ABSTRACT

Provided herein are polymer materials that find use in, for example, delivery of short-chain fatty acids. In particular, polymers are provided that form stable nanoscale structures and release their payload, for example, by cleavage of a covalent bond (e.g., via hydrolysis or enzymatic cleavage). The polymers are useful, for example, for delivery of payloads (e.g., SCFAs) to the intestine for applications in health and treatment of disease, and have broad applicability in diseases linked to changes in the human microbiota including inflammatory, autoimmune, allergic, metabolic, and central nervous system diseases, among others.

### CROSS-REFERENCE TO RELATED APPLICATIONS

This application claims the benefit of U.S. Provisional Pat. Application No. 63/329,913, filed on Apr. 12, 2022, and U.S. Provisional Pat. Application No. 63/275,260, filed on Nov. 3, 2021, both of which are incorporated by reference herein.

### FIELD

Provided herein are polymer materials that find use in, for example, delivery of short-chain fatty acids. The copolymers are provided that form stable nanoscale structures (e.g., micelles) and release their payload, for example, by cleavage of a covalent bond (e.g., via hydrolysis or enzymatic cleavage). The polymers are useful, for example, for delivery of payloads (e.g., short-chain fatty acids (SCFAs)) to the intestine for applications in health and treatment of disease, and have broad applicability in diseases linked to changes in the human microbiota including inflammatory, autoimmune, allergic, metabolic, and central nervous system diseases, among others. In particular embodiments, provided herein are prodrug polymeric micelles that find use in the delivery of short-chain fatty acids to the intestine for the promotion of gut health, establishment of healthy microbiota, treatment of immune and/or inflammatory conditions, such as inflammatory bowel disease and food allergies.

### BACKGROUND OF THE INVENTION

The gut microbiome has many effects on both mucosal and systemic health (Ref. B9; incorporated by reference in its entirety). Resident commensal bacteria play a critical role in the maintenance of mucosal homeostasis, in part through their production of short-chain fatty acids, especially butyrate (Refs. B10-B12; incorporated by reference in their entireties). Butyrate is produced by a subset of intestinal bacteria through the fermentation of dietary fiber (Ref. B13; incorporated by reference in its entirety). Butyrate is the preferred energy substrate for colonic epithelial cells and strengthens gut barrier function by stabilizing hypoxia-inducible factor and maintaining epithelial tight junctions (Refs. B12, B14; incorporated by reference in their entireties). Butyrate also promotes the production of antimicrobial peptides (AMPs), which regulate intestinal homeostasis by shaping the composition of the microbiome (Ref. B15; incorporated by reference in its entirety). To mediate its immunomodulatory functions butyrate acts via signaling through specific G protein coupled receptors or as an inhibitor of histone deacetylase activity (HDACs) (Ref. B5; incorporated by reference in its entirety). HDAC inhibition by SCFAs promotes the differentiation of colonic regulatory T cells (Tregs) (Refs. B16-B18: incorporated by reference in their entireties).

Food allergy is a prevalent and severe condition that affects over 32 million Americans (Ref. B7; incorporated by reference in its entirety). Among adults with food allergy in the US, 38% reported at least one emergency department visit related to food allergy in their lifetime (Ref. B7; incorporated by reference in its entirety). Recently, Palforzia was approved by the US FDA as an oral immunotherapy (OIT) for peanut allergy, becoming the first approved therapeutic for a food allergy (Ref. B7; incorporated by reference in its entirety). The goal of the therapy is to establish a desensitized state by exposing patients to gradually increasing doses of peanut protein. Although OIT shows efficacy in inducing desensitization to peanut antigen, it requires a prolonged period of up-dosing, during which gastrointestinal symptoms are common (Ref. B7; incorporated by reference in its entirety). Moreover, OIT is unlikely to achieve long-lasting non-responsiveness to peanut antigen in its current form⁸. Due to the adverse effects and limited efficacy of OIT, there is an urgent need to develop new therapies for food allergies.

Experiments have shown that neonatal administration of antibiotics reduces intestinal microbial diversity and impairs epithelial barrier function, resulting in increased access of food allergens to the systemic circulation (Ref. B19; incorporated by reference in its entirety). Administration of a consortium of spore-forming bacteria in the Clostridia class restored the integrity of the epithelial barrier and prevented allergic sensitization to food (Ref. B19; incorporated by reference in its entirety). Experiments have further demonstrated a causal role for bacteria present in the healthy infant microbiota in protection against cow’s milk allergy (Ref. B3; incorporated by reference in its entirety). Transfer of the microbiota from healthy, but not cow’s milk allergic (CMA), human infants into germ free (GF) mice protected against an anaphylactic response to a cow’s milk allergen. By integrating differences in the microbiome signatures present in the healthy and CMA microbiotas with the changes each induced in ileal gene expression upon colonization of GF mice, a single butyrate producing Clostridial species, Anaerostipes caccae, was identified that mimicked the effects of the healthy microbiota upon monocolonization of GF mice (Ref. B3; incorporated by reference in its entirety). Recent findings from a diverse cohort of twin children and adults concordant and discordant for food allergy validated the mouse model data with human microbiome samples. It was found that most of the operational taxonomic units (OTUs) differentially abundant between healthy and allergic twins were in the Clostridia class; the broad age range of the twins studied indicated that an early-life depletion of allergy-protective Clostridia is maintained throughout life (Ref. B2; incorporated by reference in its entirety). There is substantial interest in the use of butyrate-producing Clostridia as live biotherapeutics, but long-term engraftment of oxygen-sensitive anaerobic bacteria has proven challenging (Refs. B20-B21; incorporated by reference in their entireties).

Butyrate, produced by commensal bacteria via metabolizing dietary fiber, is known to be an agonist to G-protein coupled receptor and an inhibitor to histone deacetylase (HDAC) (Ref. A1; incorporated by reference in its entirety). Butyrate is also a preferred substrate for intestinal epithelial cells (Ref. A2; incorporated by reference in its entirety), and strengthens the gut barrier function by stabilizing hypoxia-inducible factor and maintaining tight junctions (Ref. A3; incorporated by reference in its entirety). In addition, butyrate has been demonstrated to induce the colonic regulatory T cells (Refs. A4-A6; incorporated by reference in their entireties). The important roles that butyrate plays in gut immunity make it as a good candidate drug to protect the gut immunity and to induce oral tolerance. However, butyrate, and other short-chain fatty acids, are not suitable for oral administration. Butyrate, even with enteric coating or encapsulation, possesses a foul and lasting odor and taste. As a sodium salt, orally administered butyrate is not absorbed in the part of the gut where it can have a therapeutic effect and is metabolized too rapidly to maintain a pharmacologic effect²². Previous work in murine models that demonstrated therapeutic effects of butyrate relied on high concentration, ad libitum exposure to butyrate (mM quantities in drinking water) or utilized butyrylated starches (Refs. B16-B18, B23-B25; incorporated by reference in their entireties). A more controlled and practical delivery strategy is needed to exploit the potential therapeutic benefits of butyrate clinically to treat allergic and inflammatory diseases (e.g., food allergies, inflammatory bowel disease, etc.) of the lower gastrointestinal (GI) tract.

Translating short-chain fatty acid treatments to clinical use has been challenging because degradation occurs rapidly during transit through the gut and the molecules themselves, both as free base and acid salts, are unpalatable, malodorous, and upsetting to the stomach. Enterically-coated short-chain fatty acid products are commercially available but not widely used, partly due to the aforementioned drawbacks, as well as their inability to densely pack the pharmaceutically-active short-chain fatty acid in sufficient quantities to demonstrate therapeutic effects.

Delivery systems that overcome the above limitations would be useful for all the known clinical applications of short-chain fatty acids enumerated above.

### SUMMARY OF THE INVENTION

Provided herein are prodrug polymeric micelles that find use in the delivery of short-chain fatty acids to the intestine for the promotion of gut health, establishment of healthy microbiota, treatment of immune and/or inflammatory conditions, such as inflammatory bowel disease and food allergies.

Provided herein are copolymers (e.g., random or block) that are delivery vehicles for short- to medium-chain hydrophobic or amphiphilic carboxylic acids (e.g., 3-12 carbon atoms in the chain, collectively referred to herein are short-chain fatty acids [“SCFA”s]) and functionalized derivatives of those acids. In some embodiments, the copolymers are delivery vehicles for butyrate. The polymers provide delivery of the SCFAs to the gut, including the mucosal lining of the small and large intestine, and in particular embodiments, the ileum and cecum. The SCFAs and/or their derivatives are attached to the copolymer backbone with a covalent bond, which is cleavable by hydrolysis or enzyme, thereby releasing the SCFA to have a desired therapeutic effect on human disease. The therapeutic effect is targeted at the barrier function of the intestine and the mucus layer of the gut and all diseases in which mucus layer thickness or barrier function are implicated may be treated. In some embodiments, the therapeutic effect is the promotion of gut health, establishment or maintenance of healthy gut microflora (e.g., clostridia species), treatment of inflammatory conditions (e.g., IBD), and/or treatment of immune conditions (e.g., food allergies). Exemplary human diseases that are treatable with the polymers described herein include, but are not limited to, autoimmune diseases (e.g., rheumatoid arthritis, celiac disease), allergic and atopic diseases (e.g., food allergies of all types, eosinophilic esophagitis, allergic rhinitis, allergic asthma, pet allergies, drug allergies), inflammatory conditions (e.g., inflammatory bowel disease, ulcerative colitis, Crohn’s disease), infectious diseases, metabolic disorders, diseases of the central nervous system (e.g., multiple sclerosis, Alzheimer’s disease, Parkinson’s disease), blood disorders (e.g., beta-thalassemia) colorectal cancer, diseases effecting gut motility (e.g., diarrhea), Type I diabetes, and autism spectrum disorders, among others. The copolymers are administered by any suitable route of administration (e.g., orally, rectally, etc.), and overcome the known limitations associated with the administration of short-chain fatty acids (e.g., butyrate) on their own.

Embodiments herein relate to copolymers (e.g., random or block) of (i) a monomer comprising MAA and (ii) a monomer that displays an SCFA moiety (e.g., butyrate) and is attached to the copolymer by a methacrylate or methacrylamide group, supramolecular assemblies (e.g., micelles) thereof, nanoparticles comprising such copolymers, and methods of use thereof. In some embodiments, the copolymers comprise a random, or pseudo-random distribution of the two types of monomers. In other embodiments, the copolymer is a block copolymer comprising a MAA block and a block comprising monomers that display an SCFA moiety (e.g., butyrate) and are attached to the copolymer by a methacrylate or methacrylamide group (e.g., SFCA-displaying poly(N-oxyethyl methacrylate) block, SFCA-displaying poly(N-oxyethyl methacrylamide) block, SFCA-displaying poly(N-(4-hydroxybenzoyloxy)alkyl methacrylamide) block, SFCA-displaying poly(N-(4-hydroxybenzoyloxy)alkyl methacrylate) block, etc.).

Advantages of the polymer drug delivery systems described herein, in which the pharmaceutically-active SCFAs (e.g., butyrate) are covalently attached to the polymer chain, include: masking odor of SCFAs, enhancing palatability of SCFAs, and increasing the bioavailability of SCFAs, especially in the distal gut, which are otherwise ill-suited for therapeutic use. In some embodiments, provided herein are micelles comprising the copolymers described herein. In some embodiments, micelles carrying SCFAs can further pack more densely, delivering therapeutically relevant doses of the bioactive molecule. In some embodiments, the delivery systems described herein can survive stomach transit and deliver a therapeutic payload of SCFAs targeted at the intestinal barrier upon hydrolysis, triggered by pH change, or by enzymatic cleavage, e.g., by bacterial or host esterases, and therefore represent attractive options for short-chain fatty acid delivery.

In some embodiments, provided herein are copolymers (e.g., block or random) of: (i) MAA monomers and (ii) a N-oxyalkyl methacrylamide monomer (or poly(N-oxyalkyl methacrylamide) block) with a SCFA moiety (e.g, butyrate) or other pharmaceutically-relevant small molecule attached to this block via a covalent bond.

In some embodiments, the N-oxyalkyl methacrylamide monomer (or poly(N-oxyalkyl methacrylamide) block) comprises monomers selected from the group consisting of oxymethyl methacrylamide, 2-oxyethyl methacrylamide, 3-oxypropyl methacrylamide, N-oxyisopropyl methacrylamide, 4-oxybutyl methacrylamide, N-oxyisobutyl methacrylamide, or N-oxyalkyl methacrylamide with longer or otherwise branched or substituted alkyl chains. In some embodiments, the N-oxyalkyl methacrylamide (or poly(N-oxyalkyl methacrylamide) block) comprises a linear alkyl chain of 1-20 carbons (e.g., 1, 2, 3, 4, 5, 6, 7, 8, 9, 10, 11, 12, 13, 14, 15, 16, 17, 18, 19, 20, or ranges therebetween (e.g., 2-8)). In some embodiments, the N-oxyalkyl methacrylamide (or poly(N-oxyalkyl methacrylamide) block) comprises a branched alkyl group of 1-20 carbons (e.g., 1, 2, 3, 4, 5, 6, 7, 8, 9, 10, 11, 12, 13, 14, 15, 16, 17, 18, 19, 20, or ranges therebetween (e.g., 2-8)), such as 2-methylpentyl, 3-ethylpentyl, 3,3-dimethylhexyl, 2,3-dimethylhexyl, 4-ethyl-2-methylhexyl, or any other suitable branched alkyl groups. In some embodiments, the N-oxyalkyl methacrylamide (or poly(N-oxyalkyl methacrylamide) block) comprises one or more double or triple carbon-carbon bonds (e.g., alkenyl or alkynyl instead of alkanyl). In some embodiments, the N-oxyalkyl methacrylamide (or poly(N-oxyalkyl methacrylamide) block) comprises a hetero alkyl group comprising one of the aforementioned alkyl groups (e.g., linear or branched) with one or more heteroatoms (e.g., O, S, NH, etc.) substituted for one of the carbons in the alkyl group (e.g., (CH₂)ₙX(CH₂)ₘ, wherein m and n are independently 1-10 and X is O, S, or NH). In some embodiments, the N-oxyalkyl methacrylamide (or poly(N-oxyalkyl methacrylamide) block) comprises a substituted alkyl group comprising one of the aforementioned alkyl groups (e.g., linear or branched) with one or more pendant substituent groups (e.g., OH, NH2, ═O, halogen, (e.g., Cl, F, Br, I), CN, CF3, etc.). In some embodiments, the poly(N-oxyalkyl methacrylamide) comprises a linear or branched alkyl group comprising any suitable combination of heteroatoms, pendant substituents, double bonds, etc. In particular embodiments, the N-oxyalkyl methacrylamide (or poly(N-oxyalkyl methacrylamide) block) is 2-oxyalkyl methacrylamide (or poly(2-oxyalkyl methacrylamide) block).

In some embodiments, provided herein are copolymers (e.g., block or random) of: (i) MAA monomers and (ii) a N-oxyalkyl phenol ester methacrylamide (or poly(N-oxyalkyl phenol ester methacrylamide) block) with a SCFA moiety (e.g., butyrate) or other pharmaceutically-relevant small molecule attached to this block via a covalent bond.

In some embodiments, the N-oxyalkyl 4-phenol ester methacrylamide monomer (or poly(N-oxyalkyl 4-phenol ester methacrylamide) block) comprises monomers selected from the group consisting of oxymethyl 4-phenol methacrylamide, 2-oxyethyl 4-phenol methacrylamide, 3-oxypropyl 4-phenol methacrylamide, 4-oxybutyl 4-phenol methacrylamide, or N-oxyalkyl 4-phenol methacrylamide with longer or otherwise branched or substituted alkyl chains. In some embodiments, the N-oxyalkyl 4-phenol ester methacrylamide monomer (or poly(N-oxyalkyl 4-phenol ester methacrylamide) block) comprises a linear alkyl chain of 1-20 carbons (e.g., 1, 2, 3, 4, 5, 6, 7, 8, 9, 10, 11, 12, 13, 14, 15, 16, 17, 18, 19, 20, or ranges therebetween (e.g., 2-8)). In some embodiments, the N-oxyalkyl 4-phenol ester methacrylamide monomer (or poly(N-oxyalkyl 4-phenol ester methacrylamide) block) comprises a branched alkyl group of 1-20 carbons (e.g., 1, 2, 3, 4, 5, 6, 7, 8, 9, 10, 11, 12, 13, 14, 15, 16, 17, 18, 19, 20, or ranges therebetween (e.g., 2-8)), such as 2-methylpentyl, 3-ethylpentyl, 3,3-dimethylhexyl, 2,3-dimethylhexyl, 4-ethyl-2-methylhexyl, or any other suitable branched alkyl groups. In some embodiments, the N-oxyalkyl 4-phenol ester methacrylamide monomer (or poly(N-oxyalkyl 4-phenol ester methacrylamide) block) comprises one or more double or triple carbon-carbon bonds (e.g., alkenyl or alkynyl instead of alkanyl). In some embodiments, the N-oxyalkyl 4-phenol ester methacrylamide monomer (or poly(N-oxyalkyl 4-phenol ester methacrylamide) block) comprises a hetero alkyl group comprising one of the aforementioned alkyl groups (e.g., linear or branched) with one or more heteroatoms (e.g., O, S, NH, etc.) substituted for one of the carbons in the alkyl group (e.g., (CH₂)ₙX(CH₂)ₘ, wherein m and n are independently 1-10 and X is O, S, or NH). In some embodiments, the N-oxyalkyl 4-phenol ester methacrylamide monomer (or poly(N-oxyalkyl 4-phenol ester methacrylamide) block) comprises a substituted alkyl group comprising one of the aforementioned alkyl groups (e.g., linear or branched) with one or more pendant substituent groups (e.g., OH, NH2, ═O, halogen, (e.g., Cl, F, Br, I), CN, CF3, etc.). In some embodiments, the poly(N-oxyalkyl methacrylamide) comprises a linear or branched alkyl group comprising any suitable combination of heteroatoms, pendant substituents, double bonds, etc. In particular embodiments, the N-oxyalkyl 4-phenol methacrylamide is poly(2-oxyethyl 4-phenol methacrylamide).

In some embodiments, poly(N-oxyalkyl 4-phenol methacrylamide), with or without any alkyl modifications described above, is substituted at any position on the phenol ring with moieties selected from the groups including, but not limited to, alkyl, hydroxyl, alkoxyl, amine, N-alkyl amine, carboxyl, halogen, nitro, and derivatives thereof.

In some embodiments, provided herein are copolymers (e.g., block or random) of: (i) MAA monomers and (ii) a N-oxyalkyl methacrylate monomer (or poly(N-oxyalkyl methacrylate) block) with a SCFA moiety or other pharmaceutically-relevant small molecule attached to this block via a covalent bond.

In some embodiments, the N-oxyalkyl methacrylate monomer (or poly(N-oxyalkyl methacrylate) block) comprises monomers selected from the group consisting of oxymethyl methacrylate, 2-oxyethyl methacrylate, 3-oxypropyl methacrylate, N-oxyisopropyl methacrylate, 4-oxybutyl methacrylate, N-oxyisobutyl methacrylate, or N-oxyalkyl methacrylate with longer or otherwise branched or substituted alkyl chains. In some embodiments, the N-oxyalkyl methacrylate (or poly(N-oxyalkyl methacrylate) block) comprises a linear alkyl chain of 1-20 carbons (e.g., 1, 2, 3, 4, 5, 6, 7, 8, 9, 10, 11, 12, 13, 14, 15, 16, 17, 18, 19, 20, or ranges therebetween (e.g., 2-8)). In some embodiments, the N-oxyalkyl methacrylate (or poly(N-oxyalkyl methacrylate) block) comprises a branched alkyl group of 1-20 carbons (e.g., 1, 2, 3, 4, 5, 6, 7, 8, 9, 10, 11, 12, 13, 14, 15, 16, 17, 18, 19, 20, or ranges therebetween (e.g., 2-8)), such as 2-methylpentyl, 3-ethylpentyl, 3,3-dimethylhexyl, 2,3-dimethylhexyl, 4-ethyl-2-methylhexyl, or any other suitable branched alkyl groups. In some embodiments, the N-oxyalkyl methacrylate (or poly(N-oxyalkyl methacrylate) block) comprises one or more double or triple carbon-carbon bonds (e.g., alkenyl or alkynyl instead of alkanyl). In some embodiments, the N-oxyalkyl methacrylate (or poly(N-oxyalkyl methacrylate) block) comprises a hetero alkyl group comprising one of the aforementioned alkyl groups (e.g., linear or branched) with one or more heteroatoms (e.g., O, S, NH, etc.) substituted for one of the carbons in the alkyl group (e.g., (CH₂)ₙX(CH₂)ₘ, wherein m and n are independently 1-10 and X is O, S, or NH). In some embodiments, the N-oxyalkyl methacrylate (or poly(N-oxyalkyl methacrylate) block) comprises a substituted alkyl group comprising one of the aforementioned alkyl groups (e.g., linear or branched) with one or more pendant substituent groups (e.g., OH, NH2, ═O, halogen, (e.g., Cl, F, Br, I), CN, CF3, etc.). In some embodiments, the poly(N-oxyalkyl methacrylate) comprises a linear or branched alkyl group comprising any suitable combination of heteroatoms, pendant substituents, double bonds, etc. In particular embodiments, the N-oxyalkyl methacrylate (or poly(N-oxyalkyl methacrylate) block) is 2-oxyalkyl methacrylate (or poly(2-oxyalkyl methacrylate) block).

In some embodiments, provided herein are copolymers (e.g., block or random) of: (i) a MAA monomers or block and (ii) a N-oxyalkyl phenol ester methacrylate (or poly(N-oxyalkyl phenol ester methacrylate) block) with a SCFA moiety or other pharmaceutically-relevant small molecule attached to this block via a covalent bond.

In some embodiments, the N-oxyalkyl 4-phenol ester methacrylate monomer (or poly(N-oxyalkyl 4-phenol ester methacrylate) block) comprises monomers selected from the group consisting of oxymethyl 4-phenol methacrylate, 2-oxyethyl 4-phenol methacrylate, 3-oxypropyl 4-phenol methacrylate, 4-oxybutyl 4-phenol methacrylate, or N-oxyalkyl 4-phenol methacrylate with longer or otherwise branched or substituted alkyl chains. In some embodiments, the N-oxyalkyl 4-phenol ester methacrylate monomer (or poly(N-oxyalkyl 4-phenol ester methacrylate) block) comprises a linear alkyl chain of 1-20 carbons (e.g., 1, 2, 3, 4, 5, 6, 7, 8, 9, 10, 11, 12, 13, 14, 15, 16, 17, 18, 19, 20, or ranges therebetween (e.g., 2-8)). In some embodiments, the N-oxyalkyl 4-phenol ester methacrylate monomer (or poly(N-oxyalkyl 4-phenol ester methacrylate) block) comprises a branched alkyl group of 1-20 carbons (e.g., 1, 2, 3, 4, 5, 6, 7, 8, 9, 10, 11, 12, 13, 14, 15, 16, 17, 18, 19, 20, or ranges therebetween (e.g., 2-8)), such as 2-methylpentyl, 3-ethylpentyl, 3,3-dimethylhexyl, 2,3-dimethylhexyl, 4-ethyl-2-methylhexyl, or any other suitable branched alkyl groups. In some embodiments, the N-oxyalkyl 4-phenol ester methacrylate monomer (or poly(N-oxyalkyl 4-phenol ester methacrylate) block) comprises one or more double or triple carbon-carbon bonds (e.g., alkenyl or alkynyl instead of alkanyl). In some embodiments, the N-oxyalkyl 4-phenol ester methacrylate monomer (or poly(N-oxyalkyl 4-phenol ester methacrylate) block) comprises a hetero alkyl group comprising one of the aforementioned alkyl groups (e.g., linear or branched) with one or more heteroatoms (e.g., O, S, NH, etc.) substituted for one of the carbons in the alkyl group (e.g., (CH₂)ₙX(CH₂)ₘ, wherein m and n are independently 1-10 and X is O, S, or NH). In some embodiments, the N-oxyalkyl 4-phenol ester methacrylate monomer (or poly(N-oxyalkyl 4-phenol ester methacrylate) block) comprises a substituted alkyl group comprising one of the aforementioned alkyl groups (e.g., linear or branched) with one or more pendant substituent groups (e.g., OH, NH2, ═O, halogen, (e.g., Cl, F, Br, I), CN, CF3, etc.). In some embodiments, the poly(N-oxyalkyl methacrylate) comprises a linear or branched alkyl group comprising any suitable combination of heteroatoms, pendant substituents, double bonds, etc. In particular embodiments, the N-oxyalkyl 4-phenol methacrylate is poly(2-oxyethyl 4-phenol methacrylate).

In some embodiments, poly(N-oxyalkyl 4-phenol methacrylate), with or without any alkyl modifications described above, is substituted at any position on the phenol ring with moieties selected from the groups including, but not limited to, alkyl, hydroxyl, alkoxyl, amine, N-alkyl amine, carboxyl, halogen, nitro, and derivatives thereof.

In some embodiments, a block comprises a polymer of MAA monomers. In particular embodiments, an MAA block is poly(MAA). In certain embodiments, the molecular weight of the polyMAA block is 7000-15,000 Da (e.g., 7000 Da, 8000 Da, 9000 Da, 10000 Da, 11000 Da, 12000 Da, 13000 Da, 14000 Da, 15000 Da, or ranges therebetween (e.g., 9000-14000 Da)).

In particular embodiments, the copolymer comprises a covalently-attached SCFA moiety or other pharmaceutically-relevant small molecule. In some embodiments, the SCFA moiety is selected from the group consisting of acetic acid, propionic acid, isopropionic acid, butyric acid, isobutyric acid, valeric acid, isovaleric acid, caproic acid, caprylic acid, capric acid, lauric acid, and derivatives thereof. In some embodiments, any fatty acids with an aliphatic tail of 12 or fewer carbons (e.g., 1, 2, 3, 4, 5, 6, 7, 8, 9, 10, 11, 12, or any ranges therein (e.g., 3-10) may find use in embodiments herein. In certain embodiments, the SCFA moiety is butyrate (butyric acid) or iso-butyrate (iso-butyric acid).

In some embodiments, the ratio of the MAA block to the SCFA-displaying (or other pharmaceutically-relevant-small-molecule-displaying) block is between 0.25 and 3.5 (e.g., 0.25, 0.3, 0.35, 0.4, 0.45, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, or ranges therebetween (e.g., 0.7-1.8)). In some embodiments, the ratio of the MAA monomer to the SCFA-displaying (or other pharmaceutically-relevant-small-molecule-displaying) monomer is between 0.5 and 2.0 (e.g., 0.5, 0.6, 0.7, 0.8, 0.9, 1.0, 1.1, 1.2, 1.3, 1.4, 1.5, 1.6, 1.7, 1.8, 1.9, 2.0, or ranges therebetween (e.g., 0.7-1.8)).

In some embodiments, provided herein is a polymer comprising a MAA to SCFA-displaying monomer incorporation ratio of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20 (or any ranges therebetween).

In some embodiments, a polymer comprises 20-80 percent by weight (e.g., 20 wt%, 25 wt%, 30 wt%, 35 wt%, 40 wt%, 45 wt%, 50 wt%, 55 wt%, 60 wt%, 65 wt%, 70 wt%, 75 wt%, 80 wt%, or ranges therebetween) MAA monomer. In some embodiments, a polymer comprises 20-80 percent by weight (e.g., 20 wt%, 25 wt%, 30 wt%, 35 wt%, 40 wt%, 45 wt%, 50 wt%, 55 wt%, 60 wt%, 65 wt%, 70 wt%, 75 wt%, 80 wt%, or ranges therebetween) SCFA-displaying monomer.

In some embodiments, provided herein are copolymers comprising MAA monomers (or a polyMAA block), and SCFA moieties (e.g., butyrate or iso-butyrate) or other pharmaceutically-relevant small molecule covalently attached, via a linker group, to the copolymer by a methacrylate or methacrylamide group.

In some embodiments, provided herein are supramolecular assemblies (e.g., micelles) comprising a plurality of the copolymers (e.g., comprising SCFAs or other small molecular cargo) described herein (e.g., dispersed in a liquid). In some embodiments, an assembly is a nanoparticle between 10-1000 nm in diameter (e.g., 10, 20, 50, 100, 200, 500, 1000 nm, or ranges therebetween (e.g., 50-500 nm)). In certain embodiments, the plurality of block copolymers comprises linear and branched copolymers self-assembled or covalently linked to form the nanoparticle. In other embodiments, the assembly is a micelle. In yet other embodiments, the supramolecular assemblies (e.g., micelles) are isolated (e.g., as a powder) and redispersed (e.g., in a liquid).

In some embodiments, provided herein are methods of delivering a target molecule (e.g., SCFA) to a subject (e.g., a human subject, a male subject, a female subject, etc.), the method comprising providing a supramolecular assembly (e.g., micelle) of the copolymers described herein, wherein the supramolecular assembly (e.g., micelle) comprises the target molecule (e.g., SCFA); and contacting the subject with the supramolecular assembly (e.g., micelle), thereby delivering the target molecule to the subject. In some embodiments, a composition (e.g., pharmaceutical composition) comprising the block copolymers described herein and/or supramolecular assemblies (e.g., micelles) thereof are administered to a subject by any suitable route of administration. In some embodiments, the target molecule (e.g., SCFA) is covalently attached to the supramolecular assembly (e.g., micelle). In particular embodiments, the supramolecular assembly (e.g., micelle) is contacted (e.g., administered) orally when given to the subject. In some embodiments, the supramolecular assembly (e.g., micelle) is dispersed in a liquid carrier when contacted with the subject. In other embodiments, the supramolecular assembly (e.g., micelle is a solid when contacted with the subject. In some embodiments the supramolecular assembly (e.g., micelle) is for use as a medicament.

In some embodiments, provided herein is the use of a supramolecular assembly (e.g., micelle) of the copolymers described herein in the manufacture of a medicament.

In some embodiments, provided herein are pharmaceutical compositions comprising the supramolecular assemblies (e.g., micelles) described herein. In particular embodiments, a supramolecular assembly (e.g., micelle) is combined with a pharmaceutically acceptable carrier (e.g., considered to be safe and effective) and is administered to a subject (e.g., without causing undesirable biological side effects or unwanted interactions).

In some embodiments, provided herein are compositions comprising a copolymer of (i) a monomer comprising methacrylic acid (MAA) and (ii) a monomer of formula (I):

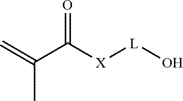

, wherein X is O, NH, or S; wherein L is a linker selected from an alkyl chain, an heteroalkyl chain, a substituted alkyl chain, or a substituted heteroalkyl chain; wherein the copolymer displays one or more short-chain fatty acid (SCFA) moieties.

In some embodiments, provided herein are compositions comprising a copolymer of (i) a monomer comprising methacrylic acid (MAA) and (ii) a monomer of formula (II):

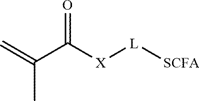

, wherein X is O, NH, or S; wherein L is a linker selected from an alkyl chain, an heteroalkyl chain, a substituted alkyl chain, or a substituted heteroalkyl chain; and wherein SCFA is a short-chain fatty acid.

In some embodiments, L of formula (I) or formula (II) is (CH₂)ₙ, wherein n is 1-16 (e.g., 1, 2, 3, 4, 5, 6, 7, 8, 9, 10, 11, 12, 13, 14, 15, 16, or ranges therebetween). In some embodiments, L is (CH₂)ₙO(CO)-benzene. In some embodiments, the SCFA is covalently attached to the monomer of formula (I). In some embodiments, the SCFA attached to the monomer of formula (I) comprises formula (II):

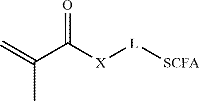

. In some embodiments, the SCFA attached to the monomer of formula (I) or formula (II) comprises formula (III):

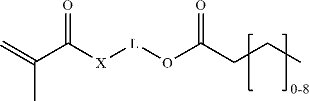

. In some embodiments, the SCFA is selected from the group consisting of acetic acid, propionic acid, isopropionic acid, butyric acid, isobutyric acid, valeric acid, isovaleric acid, caproic acid, caprylic acid, capric acid, lauric acid, branched versions thereof, and derivatives thereof. In some embodiments, the SCFA is butyric acid.

In some embodiments, the copolymer is a block copolymer comprising an MAA block and a block of formula (I) or formula (II). In some embodiments, the block copolymer

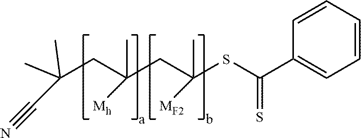

comprises the formula (IV) ; wherein Mₕ comprises MAA, M_{F2} is the side chain of the monomer of Formula (II):

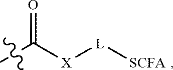

wherein a is 1-1000 (e.g., 10, 20, 30, 40, 50, 60, 70, 80, 90, 100, 125, 133, 150, 175, 200, 250, 300, 400, 500, 600, 700, 800, 900, 1000, or ranges therebetween) and b is 1-1000 (e.g., 10, 20, 30, 40, 50, 60, 70, 80, 90, 100, 125, 133, 150, 175, 200, 250, 300, 400, 500, 600, 700, 800, 900, 1000, or ranges therebetween).

In some embodiments, the copolymer is a random copolymer. In some embodiments, the random copolymer comprises formula (V):

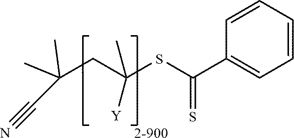

; wherein each Y is independently selected from the side chain of a polymer formed from formula (II):

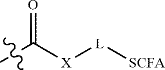

, and the side chain of MAA:

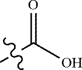

. In some embodiments, there are 2, 5, 10, 20, 30, 40, 50, 75, 100, 125, 150, 175, 200, 250, 300, 350, 400, 450, 500, 550, 600, 650, 700, 750, 800, 850, 900, or ranges therebetween of the repeated Y-displaying groups.

In some embodiments, the monomer of formula (II) comprises N-butanoyloxyalkyl methacrylamide. In some embodiments, the N-butanoyloxyalkyl methacrylamide monomer is 2-butanoyloxyethyl methacrylamide. In some embodiments, the copolymer is a block copolymer and comprises formula (VI):

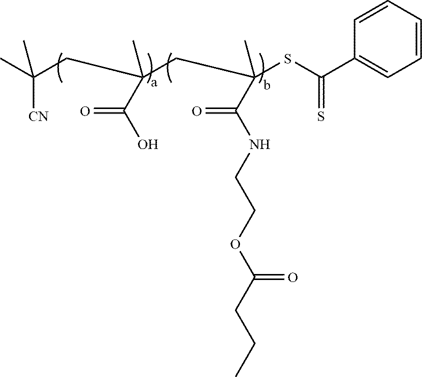

; wherein a and b are independently 1-1000 (e.g., 10, 20, 30, 40, 50, 60, 70, 80, 90, 100, 125, 133, 150, 175, 200, 250, 300, 400, 500, 600, 700, 800, 900, 1000, or ranges therebetween). In some embodiments, the copolymer is a random copolymer and comprises formula (V):

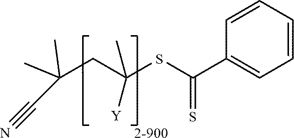

; wherein each Y is independently selected from (i) the side chain of MAA:

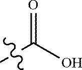

, and (ii) the side chain of poly(2-butanoyloxyethyl methacrylamide). In some embodiments, there are 2, 5, 10, 20, 30, 40, 50, 75, 100, 125, 150, 175 200, 250, 300, 350, 400, 450, 500, 550, 600, 650, 700, 750, 800, 850, 900, or ranges therebetween of the repeated Y-displaying groups.

In some embodiments, the monomer of formula (II) comprises an N-butanoyloxyalkyl methacrylate. In some embodiments, the N-butanoyloxyalkyl methacrylate monomer is an 2-butanoyloxyethyl methacrylate. In some embodiments, the copolymer is a block copolymer and comprises formula (VII):

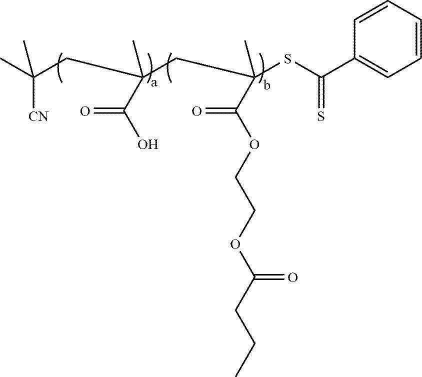

; wherein a and b are independently 1-1000 (e.g., 10, 20, 30, 40, 50, 60, 70, 80, 90, 100, 125, 133, 150, 175, 200, 250, 300, 400, 500, 600, 700, 800, 900, 1000, or ranges therebetween). In some embodiments, the copolymer is a random copolymer and comprises formula (V):

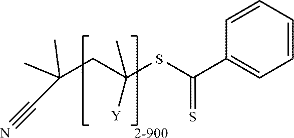

; wherein each Y is independently selected from (i) the side chain of MAA:

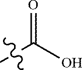

, and (ii) the side chain of poly(2-butanoyloxyethyl methacrylate). In some embodiments, there are 2, 5, 10, 20, 30, 40, 50, 75, 100, 125, 150, 175, 200, 250, 300, 350, 400, 450, 500, 550, 600, 650, 700, 750, 800, 850, 900, or ranges therebetween of the repeated Y-displaying groups.

In some embodiments, the monomer of formula (II) comprises an N-(4-butanoyloxybenzoyloxy)alkyl methacrylate. In some embodiments, the N-(4-butanoyloxybenzoyloxy)alkyl methacrylate monomer is 2-(4-butanoyloxybenzoyloxy)ethyl methacrylate. In some embodiments, the copolymer is a block copolymer and comprises formula (VIII):

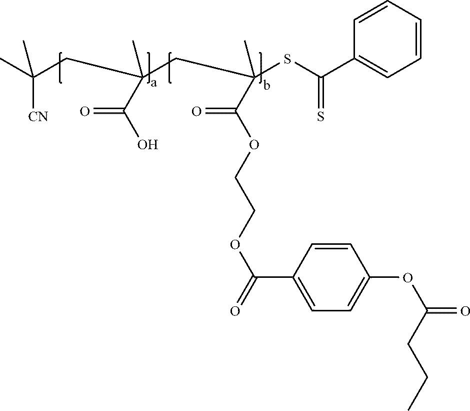

; wherein a and b are independently 1-1000 (e.g., 10, 20, 30, 40, 50, 60, 70, 80, 90, 100, 125, 133, 150, 175, 200, 250, 300, 400, 500, 600, 700, 800, 900, 1000, or ranges therebetween). In some embodiments, the copolymer is a random copolymer and comprises formula (V):

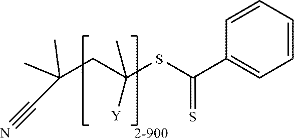

; wherein each Y is independently selected from (i) the side chain of MAA:

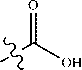

and (ii) the side chain of poly(2-(4-butanoyloxybenzoyloxy)ethyl methacrylate):

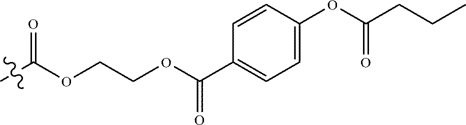

. In some embodiments, there are 2, 5, 10, 20, 30, 40, 50, 75, 100, 125, 150, 175, 200, 250, 300, 350, 400, 450, 500, 550, 600, 650, 700, 750, 800, 850, 900, or ranges therebetween of the repeated Y-displaying groups.

In some embodiments, the monomer of formula (II) comprises an N-(4-butanoyloxybenzoyloxy)alkyl methacrylamide. In some embodiments, the N-(4-butanoyloxybenzoyloxy)alkyl methacrylamide monomer is 2-(4-butanoyloxybenzoyloxy)ethyl methacrylamide. In some embodiments, the copolymer is a block copolymer and comprises formula (IX):

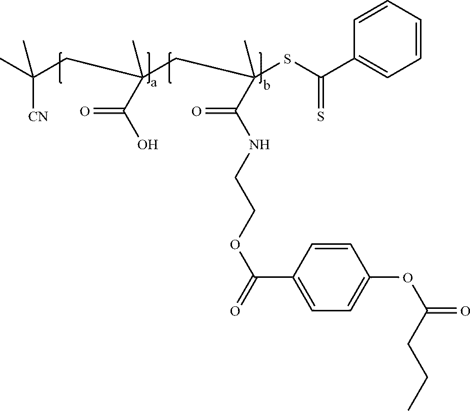

; wherein a and b are independently 1-1000 (e.g., 10, 20, 30, 40, 50, 60, 70, 80, 90, 100, 125, 133, 150, 175, 200, 250, 300, 400, 500, 600, 700, 800, 900, 1000, or ranges therebetween). In some embodiments, the copolymer is a random copolymer and comprises formula (V):

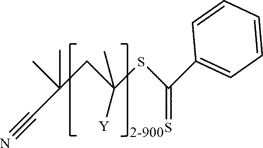

; wherein each Y is independently selected from (i) the side chain of MAA:

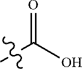

, and (ii) the side chain of poly(2-(4-butanoyloxybenzoyloxy)ethyl methacrylamide):

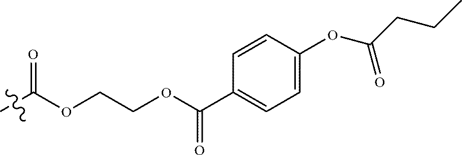

. In some embodiments, there are 2, 5, 10, 20, 30, 40, 50, 75, 100, 125, 150, 175, 200, 250, 300, 350, 400, 450, 500, 550, 600, 650, 700, 750, 800, 850, 900, or ranges therebetween of the repeated Y-displaying groups.

In some embodiments, compositions herein comprise a second copolymer (e.g., in addition to an MAA-containing copolymer) or micelles thereof, the second copolymer comprising: (i) a monomer comprising N-(2-hydroxyethyl) methacrylamide (HPMA) and (ii) a monomer of formula (II)₂:

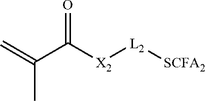

, wherein X₂ is O, NH, or S; wherein L₂ is a linker selected from an alkyl chain, an heteroalkyl chain, a substituted alkyl chain, or a substituted heteroalkyl chain; and wherein SCFA₂ is a short-chain fatty acid. In some embodiments, L₂ is (CH₂)ₙ, wherein n is 1-16. In some embodiments, L₂ is (CH₂)ₙO(CO)-benzene. In some embodiments, the monomer of formula (II)₂ comprises formula (III)₂:

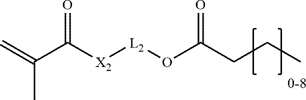

In some embodiments, the SCFA₂ is selected from the group consisting of acetic acid, propionic acid, isopropionic acid, butyric acid, isobutyric acid, valeric acid, isovaleric acid, caproic acid, caprylic acid, capric acid, lauric acid, branched versions thereof, and derivatives thereof. In some embodiments, the SCFA₂ is butyric acid. In some embodiments, the second copolymer comprises 10-80 wt% butyric acid. In some embodiments, the second copolymer is a block copolymer comprising a HPMA block and a block of formula (II)₂. In some embodiments, the second copolymer is a random copolymer. For example, in some embodiments, the second copolymer comprises the formula (V)₂:

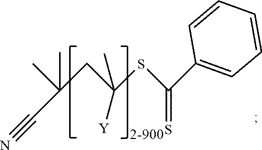

wherein each Y₂ is independently selected from the side chain of formula (II)₂:

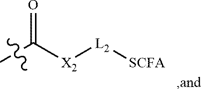

the side chain of polyHPMA:

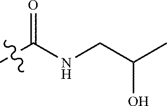

. In some embodiments, the monomer of formula (II)₂ comprises N-butanoyloxyalkyl methacrylamide. In some embodiments, the N-butanoyloxyalkyl methacrylamide monomer is 2-butanoyloxyethyl methacrylamide.

In some embodiments, the second copolymer is a block copolymer and comprises formula (VI)₂:

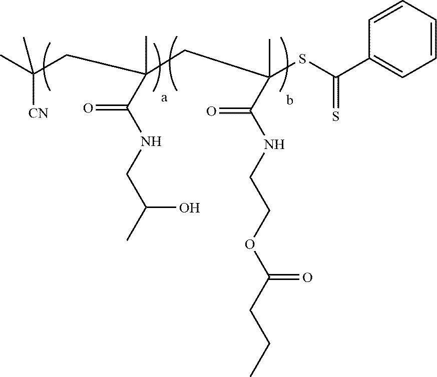

; wherein a and b are independently 1-1000. In some embodiments, the second copolymer is a random copolymer and comprises formula (V)₂:

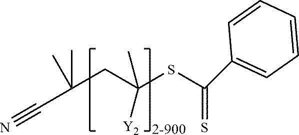

; wherein each Y₂ is independently selected from (i) the side chain of polyHPMA:

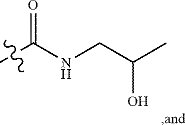

(ii) the side chain of poly(2-butanoyloxyethyl methacrylamide):

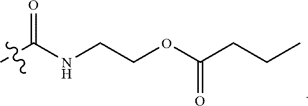

In some embodiments, the monomer of formula (II)₂ comprises an N-butanoyloxyalkyl methacrylate. In some embodiments, the N-butanoyloxyalkyl methacrylate monomer is a 2-butanoyloxyethyl methacrylate. In some embodiments, the second copolymer is a block copolymer and comprises formula (VII)₂:

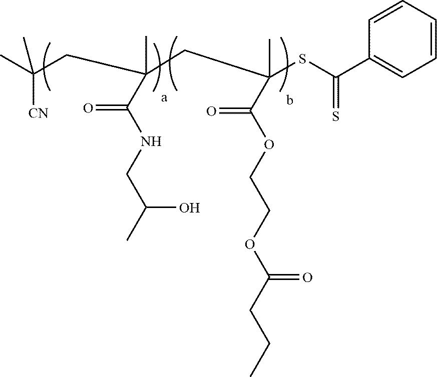

; wherein a and b are independently 1-1000. In some embodiments, the second copolymer is a random copolymer and comprises formula (V)₂:

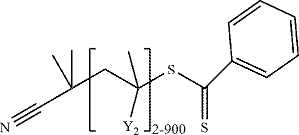

; wherein each Y₂ is independently selected from (i) the sidechain of polyHPMA:

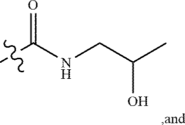

(ii) an 2-butanoyloxyethyl methacrylate. In some embodiments, the monomer of formula (II)₂ comprises an N-(4-butanoyloxybenzoyloxy)alkyl methacrylate monomer. In some embodiments, the N-(4-butanoyloxybenzoyloxy)alkyl methacrylate monomer is 2-(4-butanoyloxybenzoyloxy)ethyl methacrylate. In some embodiments, the second copolymer is a block copolymer and comprises formula (VIII)₂:

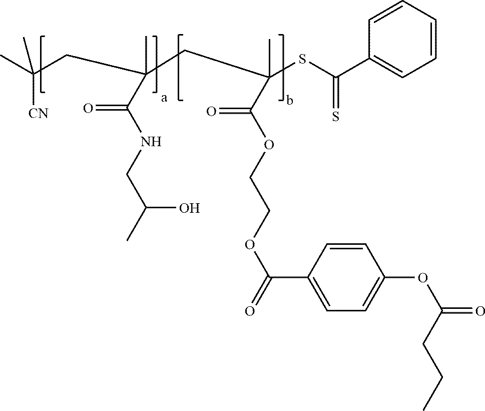

; wherein a and b are independently 1-1000. In some embodiments, the second copolymer is a random copolymer and comprises formula (V)₂:

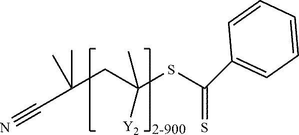

; wherein each Y₂ is independently selected from (i) the side chain of polyHPMA:

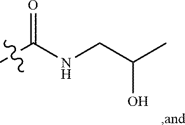

(ii) the side chain of poly(2-(4-butanoyloxybenzoyloxy)ethyl methacrylate):

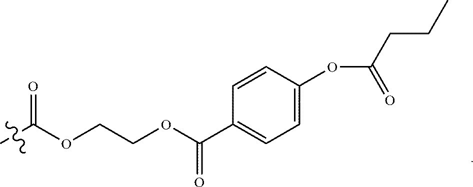

In some embodiments, the monomer of formula (II)₂ comprises an N-(4-butanoyloxybenzoyloxy)alkyl methacrylamide monomer. In some embodiments, the N-(4-butanoyloxybenzoyloxy)alkyl methacrylamide monomer is 2-(4-butanoyloxybenzoyloxy)ethyl methacrylamide. In some embodiments, the second copolymer is a block copolymer and comprises formula (IX)₂:

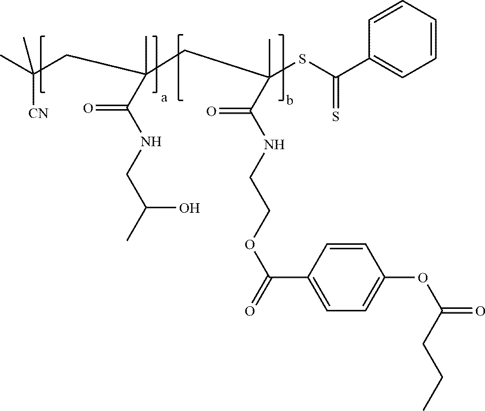

; wherein a and b are independently 1-1000. In some embodiments, the second copolymer is a random copolymer and comprises formula (V)₂:

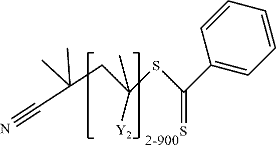

; wherein each Y₂ is independently selected from (i) the side chain of polyHPMA:

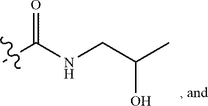

(ii) the side chain of poly(2-(4-butanoyloxybenzoyloxy)ethyl methacrylamide):

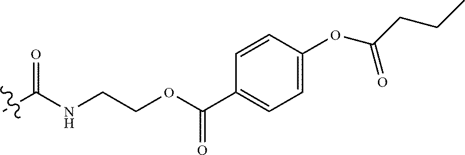

In some embodiments, provided herein are compositions comprising: (a) poly(2-hydroxypropyl methacrylamide)-b-poly(N-(2-alkanoyloxyethyl) methacrylamide) (pHPMA-b-pAMA); and (b) poly(methacrylic acid)-b-poly(N-(2-alkanoyloxyethyl) methacrylamide (pMAA-b-pAMA). In some embodiments, the pMAA-b-pAMA is present as negatively-charged micelles. In some embodiments, the pHPMA-b-pAMA is present as neutrally-charged micelles. In some embodiments, the pMAA-b-pAMA comprises one or more of poly(methacrylic acid)-b-poly(N-(2-methanoyloxyethyl) methacrylamide) (pMAA-b-pMMA), poly(methacrylic acid)-b-poly(N-(2-alkanoyloxyethyl) methacrylamide) (pMAA-b-pEMA), poly(methacrylic acid)-b-poly(N-(2-propanoyloxyethyl) methacrylamide) (pMAA-b-pPMA), poly(methacrylic acid)-b-poly(N-(2-butanoyloxyethyl) methacrylamide) (pMAA-b-pBMA), poly(methacrylic acid)-b-poly(N-(2-pentanoyloxyethyl) methacrylamide) (pMAA-b-pPeMA), poly(methacrylic acid)-b-poly(N-(2-hexanoyloxyethyl) methacrylamide) (pMAA-b-pHMA), or longer SCFA-containing copolymers. In some embodiments, the pHPMA-b-pAMA comprises one or more of poly(2-hydroxypropyl methacrylamide)-b-poly(N-(2-methanoyloxyethyl) methacrylamide) (pHPMA-b-pMMA), poly(2-hydroxypropyl ethacrylamide)-b-poly(N-(2-alkanoyloxyethyl) methacrylamide) (pHPMA-b-pEMA), poly(2-hydroxypropyl methacrylamide)-b-poly(N-(2-propanoyloxyethyl) methacrylamide) (pHPMA-b-pPMA), poly(2-hydroxypropyl methacrylamide)-b-poly(N-(2-butanoyloxyethyl) methacrylamide) (pHPMA-b-pBMA), poly(2-hydroxypropyl methacrylamide)-b-poly(N-(2-pentanoyloxyethyl) methacrylamide) (pHPMA-b-pPeMA), poly(2-hydroxypropyl methacrylamide)-b-poly(N-(2-hexanoyloxyethyl) methacrylamide) (pHPMA-b-pHMA), or longer SCFA-containing copolymers. In some embodiments, provided herein are pharmaceutical composition comprising the compositions described herein and a pharmaceutically-acceptable carrier. In some embodiments, provided herein are foods or nutraceutical compositions comprising compositions described herein and an edible carrier. In some embodiments, provided herein are methods comprising administering to a subject a pharmaceutical composition, food or nutraceutical composition described herein to a subject in need thereof. In some embodiments, the subject suffers from food allergies. In some embodiments, the subject suffers from dysbiosis. In some embodiments, the subject has been administered antibiotics. In some embodiments, the method results in an increase in the abundance and/or relative abundance of Enterococcus, Coprobacter, and Clostridium Cluster XIVa. In some embodiments, the method results in an increase in the abundance and/or relative abundance of bacteria of the family Lachnospiraceae. In some embodiments, the method results in an increase in the abundance and/or relative abundance of Clostridium Cluster XIVa, IV and/or XVIII bacteria. In some embodiments, methods result in improved intestinal barrier function, reduced inflammation, improved physician scores, improved patient-reported outcomes, and/or reduced sensitivity to allergens. In some embodiments, the method results in increased production of butyrate and other beneficial metabolites by the gut microflora of the subject.

In some embodiments, provided herein are methods of establishing a healthy gut microflora in a subject comprising administering a composition comprising (a) poly(2-hydroxypropyl methacrylamide)-b-poly(N-(2-alkanoyloxyethyl) methacrylamide) (pHPMA-b-pAMA); or (b) poly(methacrylic acid)-b-poly(N-(2-alkanoyloxyethyl) methacrylamide (pMAA-b-pAMA) to a subject in need thereof. In some embodiments, the pHPMA-b-pAMA comprises one or more of poly(2-hydroxypropyl methacrylamide)-b-poly(N-(2-methanoyloxyethyl) methacrylamide) (pHPMA-b-pMMA), poly(2-hydroxypropyl ethacrylamide)-b-poly(N-(2-alkanoyloxyethyl) methacrylamide) (pHPMA-b-pEMA), poly(2-hydroxypropyl methacrylamide)-b-poly(N-(2-propanoyloxyethyl) methacrylamide) (pHPMA-b-pPMA), poly(2-hydroxypropyl methacrylamide)-b-poly(N-(2-butanoyloxyethyl) methacrylamide) (pHPMA-b-pBMA), poly(2-hydroxypropyl methacrylamide)-b-poly(N-(2-pentanoyloxyethyl) methacrylamide) (pHPMA-b-pPeMA), poly(2-hydroxypropyl methacrylamide)-b-poly(N-(2-hexanoyloxyethyl) methacrylamide) (pHPMA-b-pHMA), or longer SCFA-containing copolymers. In some embodiments, the pMAA-b-pAMA comprises one or more of poly(methacrylic acid)-b-poly(N-(2-methanoyloxyethyl) methacrylamide) (pMAA-b-pMMA), poly(methacrylic acid)-b-poly(N-(2-alkanoyloxyethyl) methacrylamide) (pMAA-b-pEMA), poly(methacrylic acid)-b-poly(N-(2-propanoyloxyethyl) methacrylamide) (pMAA-b-pPMA), poly(methacrylic acid)-b-poly(N-(2-butanoyloxyethyl) methacrylamide) (pMAA-b-pBMA), poly(methacrylic acid)-b-poly(N-(2-pentanoyloxyethyl) methacrylamide) (pMAA-b-pPeMA), poly(methacrylic acid)-b-poly(N-(2-hexanoyloxyethyl) methacrylamide) (pMAA-b-pHMA), or longer SCFA-containing copolymers. In some embodiments, the subject suffers from dysbiosis. In some embodiments, the subject has been administered antibiotics. In some embodiments, the method results in an increase in the relative abundance of Enterococcus, Coprobacter, and Clostridium Cluster XIVa. In some embodiments, the method results in an increase in the abundance and/or relative abundance of bacteria of the family Lachnospiraceae. In some embodiments, the method results in an increase in the abundance and/or relative abundance of Clostridium Cluster XIVa, IV and/or XVIII bacteria. In some embodiments, methods result in improved intestinal barrier function, reduced inflammation, improved physician scores, improved patient-reported outcomes, and/or reduced sensitivity to allergens. In some embodiments, the method results in increased production of butyrate and other beneficial metabolites by the gut microflora of the subject. In some embodiments, provided herein are methods comprising (a) detecting bacteria and/or a bacterial metabolite in the stool of a subject; and (b) administering a composition comprising (i) poly(2-hydroxypropyl methacrylamide)-b-poly(N-(2-alkanoyloxyethyl) methacrylamide) (pHPMA-b-pAMA); or (ii) poly(methacrylic acid)-b-poly(N-(2-alkanoyloxyethyl) methacrylamide (pMAA-b-pAMA) to the subject. In some embodiments, the pHPMA-b-pAMA comprises one or more of poly(2-hydroxypropyl methacrylamide)-b-poly(N-(2-methanoyloxyethyl) methacrylamide) (pHPMA-b-pMMA), poly(2-hydroxypropyl ethacrylamide)-b-poly(N-(2-alkanoyloxyethyl) methacrylamide) (pHPMA-b-pEMA), poly(2-hydroxypropyl methacrylamide)-b-poly(N-(2-propanoyloxyethyl) methacrylamide) (pHPMA-b-pPMA), poly(2-hydroxypropyl methacrylamide)-b-poly(N-(2-butanoyloxyethyl) methacrylamide) (pHPMA-b-pBMA), poly(2-hydroxypropyl methacrylamide)-b-poly(N-(2-pentanoyloxyethyl) methacrylamide) (pHPMA-b-pPeMA), poly(2-hydroxypropyl methacrylamide)-b-poly(N-(2-hexanoyloxyethyl) methacrylamide) (pHPMA-b-pHMA), or longer SCFA-containing copolymers. In some embodiments, the pMAA-b-pAMA comprises one or more of poly(methacrylic acid)-b-poly(N-(2-methanoyloxyethyl) methacrylamide) (pMAA-b-pMMA), poly(methacrylic acid)-b-poly(N-(2-alkanoyloxyethyl) methacrylamide) (pMAA-b-pEMA), poly(methacrylic acid)-b-poly(N-(2-propanoyloxyethyl) methacrylamide) (pMAA-b-pPMA), poly(methacrylic acid)-b-poly(N-(2-butanoyloxyethyl) methacrylamide) (pMAA-b-pBMA), poly(methacrylic acid)-b-poly(N-(2-pentanoyloxyethyl) methacrylamide) (pMAA-b-pPeMA), poly(methacrylic acid)-b-poly(N-(2-hexanoyloxyethyl) methacrylamide) (pMAA-b-pHMA), or longer SCFA-containing copolymers.In some embodiments, the composition is administered if it is determined that the subject suffers from dysbiosis or a gut metabolite deficiency. In some embodiments, detecting is performed before and/or after the administration of the composition. In some embodiments, detecting is used to determine whether continued administration of the composition is beneficial to the subject. In some embodiments, detecting is used to determine proper dosing of the composition.

In some embodiments, provided herein are methods comprising: (a) administering a first dose of a composition comprising (i) poly(2-hydroxypropyl methacrylamide)-b-poly(N-(2-alkanoyloxyethyl) methacrylamide) (pHPMA-b-pAMA); and/or (ii) poly(methacrylic acid)-b-poly(N-(2-alkanoyloxyethyl) methacrylamide (pMAA-b-pAMA) to a subject; (b) administering a second lower dose of the composition to the subject. In some embodiments, the pHPMA-b-pAMA comprises one or more of poly(2-hydroxypropyl methacrylamide)-b-poly(N-(2-methanoyloxyethyl) methacrylamide) (pHPMA-b-pMMA), poly(2-hydroxypropyl ethacrylamide)-b-poly(N-(2-alkanoyloxyethyl) methacrylamide) (pHPMA-b-pEMA), poly(2-hydroxypropyl methacrylamide)-b-poly(N-(2-propanoyloxyethyl) methacrylamide) (pHPMA-b-pPMA), poly(2-hydroxypropyl methacrylamide)-b-poly(N-(2-butanoyloxyethyl) methacrylamide) (pHPMA-b-pBMA), poly(2-hydroxypropyl methacrylamide)-b-poly(N-(2-pentanoyloxyethyl) methacrylamide) (pHPMA-b-pPeMA), poly(2-hydroxypropyl methacrylamide)-b-poly(N-(2-hexanoyloxyethyl) methacrylamide) (pHPMA-b-pHMA), or longer SCFA-containing copolymers. In some embodiments, the pMAA-b-pAMA comprises one or more of poly(methacrylic acid)-b-poly(N-(2-methanoyloxyethyl) methacrylamide) (pMAA-b-pMMA), poly(methacrylic acid)-b-poly(N-(2-alkanoyloxyethyl) methacrylamide) (pMAA-b-pEMA), poly(methacrylic acid)-b-poly(N-(2-propanoyloxyethyl) methacrylamide) (pMAA-b-pPMA), poly(methacrylic acid)-b-poly(N-(2-butanoyloxyethyl) methacrylamide) (pMAA-b-pBMA), poly(methacrylic acid)-b-poly(N-(2-pentanoyloxyethyl) methacrylamide) (pMAA-b-pPeMA), poly(methacrylic acid)-b-poly(N-(2-hexanoyloxyethyl) methacrylamide) (pMAA-b-pHMA), or longer SCFA-containing copolymers. In some embodiments, the first dose is administered multiple times over a first time span before the second lower dose is administered. In some embodiments, the first dose is administered twice daily, once daily, or once weekly of the first time span. In some embodiments, the first time span is one (1) week, two (2) weeks, three (3) weeks, one (1) month, two (months), four (4) months, six (6) months, one (1) year, or more or ranges therebetween. In some embodiments, the first dose contains 1-40 g (e.g., 1 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 31 g, 32 g, 33 g, 34 g, 35 g, 36 g, 37 g, 38 g, 39 g, 40 g, or ranges therebetween) of (i) poly(2-hydroxypropyl methacrylamide)-b-poly(N-(2-butanoyloxyethyl) methacrylamide (pHPMA-b-pBMA); and/or (ii) poly(methacrylic acid)-b-poly(N-(2-butanoyloxyethyl) methacrylamide (pMAA-b-pBMA). In some embodiments, the second lower dose is between one tenth (⅒) and one half (½) of the first dose (e.g., 0.1X, 0.2X, 0.3X, 0.4X, 0.5X, or ranges therebetween). In some embodiments, step (b) is performed following performing step (a) for a predetermined time span (e.g., one week, two weeks, one month two months, three months, four months, five month, six months. In some embodiments, step (b) is performed following an assessment of gut microflora of the subject. In some embodiments, the subject suffered from dysbiosis prior to step (a). In some embodiments, step (b) is performed following an assessment of levels of one or more gut metabolites of the subject. In some embodiments, one or more gut metabolites comprises a short chain fatty acid. In some embodiments, the short chain fatty acid comprises butyrate. In some embodiments, methods further comprise one or more steps of assessing gut microflora of the subject and/or assessing levels of one or more gut metabolites of the subject prior to step (a), between steps (a) and (b), and/or following step (b).

In some embodiments, provided herein are compositions comprising a first micelle of a first copolymer of methacrylic acid (MAA) and N-(2-alkanoyloxyethyl) methacrylamide (AMA). In some embodiments, the copolymer is a block copolymer having the structure:

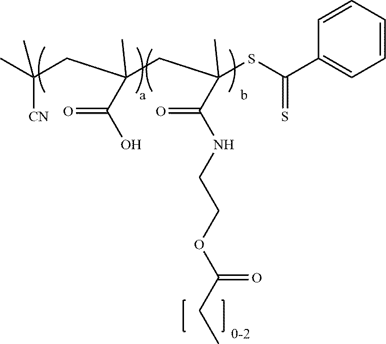

; wherein a and b are independently 1-1000. In some embodiments, the copolymer is a random copolymer having the structure:

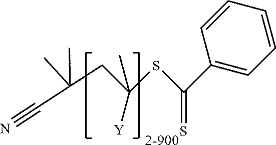

; wherein each Y is independently selected from:

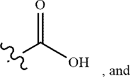

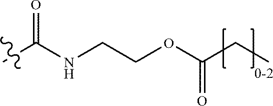

. In some embodiments, the copolymer is a block copolymer having the structure:

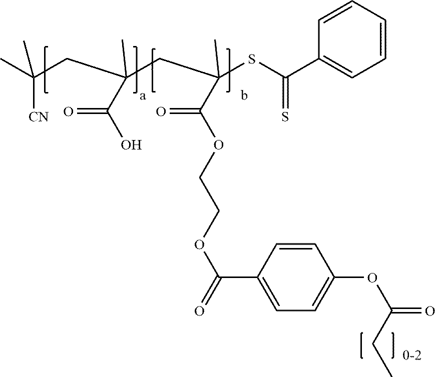

; wherein a and b are independently 1-1000. In some embodiments, the copolymer is a random copolymer having the structure:

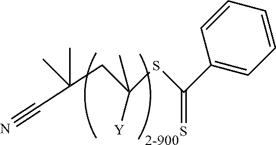

; wherein each Y is independently selected from:

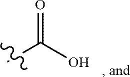

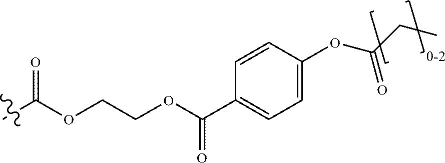

In some embodiments, the copolymer is a block copolymer having the structure:

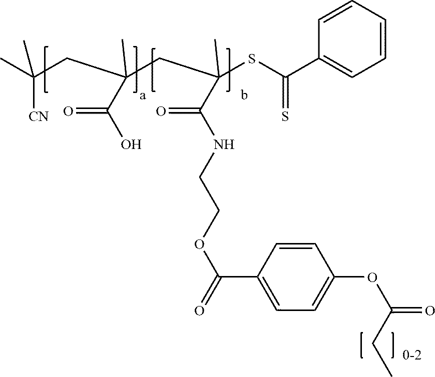

; wherein a and b are independently 1-1000. In some embodiments, the copolymer is a random copolymer having the structure:

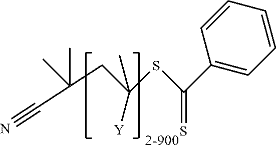

; wherein each Y is independently selected from:

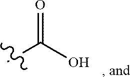

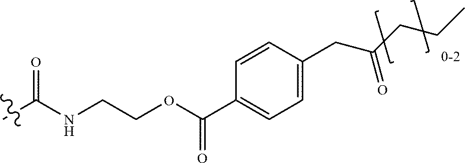

In some embodiments, the copolymer is a block copolymer and has the structure:

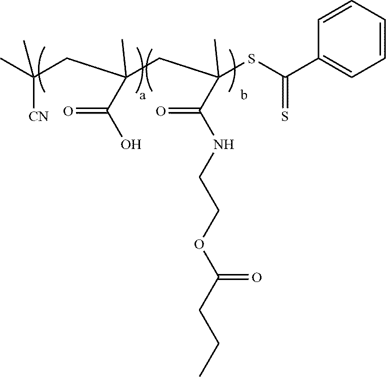

; wherein a and b are independently 1-1000. In some embodiments, the copolymer is a random copolymer and has the structure

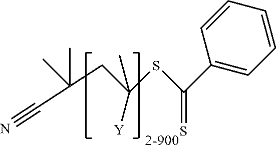

; wherein each Y is independently selected from:

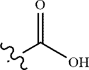

, and

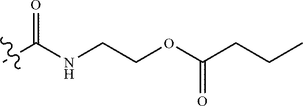

In some embodiments, the copolymer is a block copolymer having the structure:

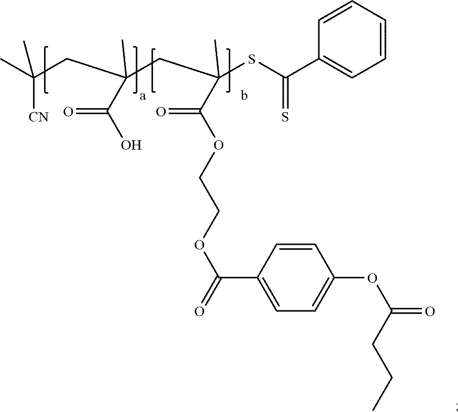

wherein a and b are independently 1-1000. In some embodiments, the copolymer is a random copolymer and has the structure:

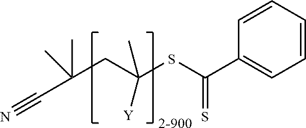

; wherein each Y is independently selected from:

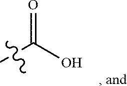

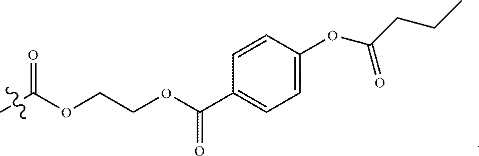

In some embodiments, the copolymer is a block copolymer having the structure:

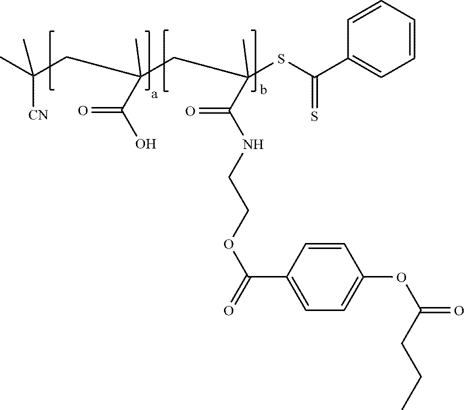

; wherein a and b are independently 1-1000. In some embodiments, the copolymer is a random copolymer having the structure:

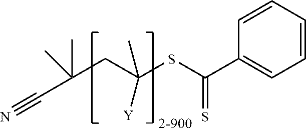

; wherein each Y is independently selected from:

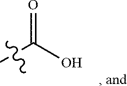

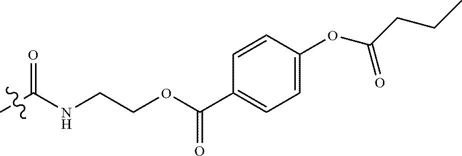

In some embodiments, a composition further comprises a second micelle of a second copolymer of 2-hydroxypropyl methacrylamide (HPMA) and N-(2-alkanoyloxyethyl) methacrylamide (AMA). In some embodiments, In some embodiments, the second copolymer is a block copolymer having the structure:

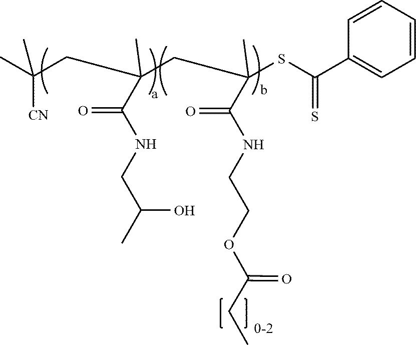

; wherein a and b are independently 1-1000. In some embodiments, the second copolymer is a random copolymer having the structure:

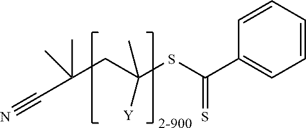

; wherein each Y is independently selected from:

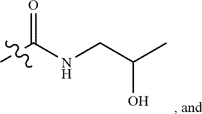

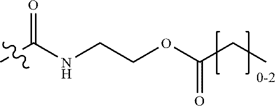

In some embodiments, the second copolymer is a block copolymer having the structure:

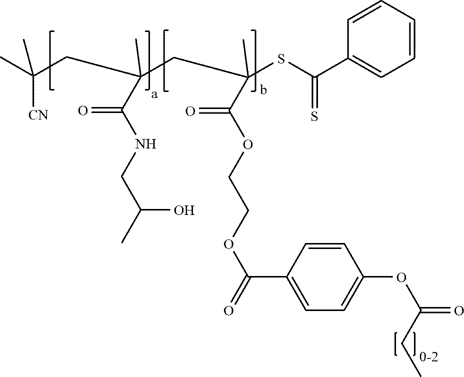

; wherein a and b are independently 1-1000. In some embodiments, the second copolymer is a random copolymer having the structure:

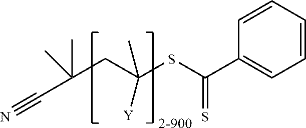

; wherein each Y is independently selected from:

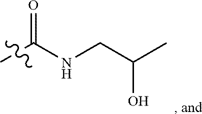

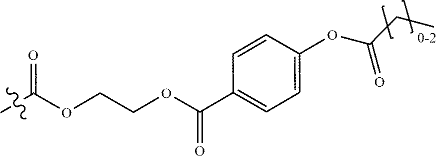

In some embodiments, the second copolymer is a block copolymer having the structure:

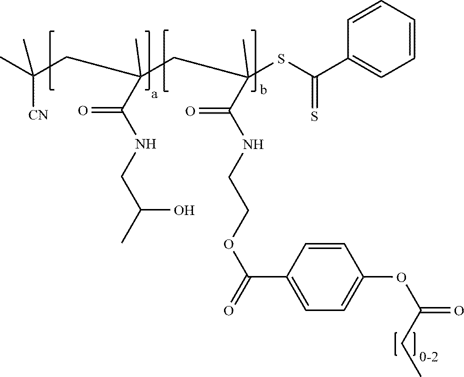

; wherein a and b are independently 1-1000.

In some embodiments, the second copolymer is a random copolymer having the structure:

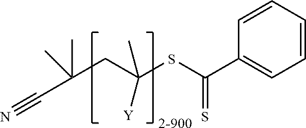

; wherein each Y is independently selected from:

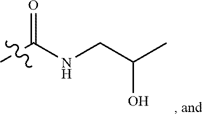

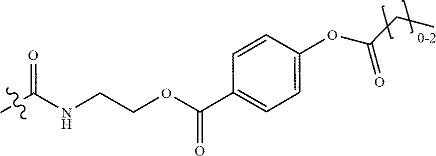

In some embodiments, the second copolymer is a block copolymer and has the structure:

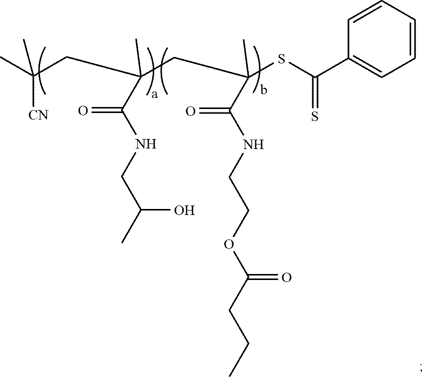

wherein a and b are independently 1-1000.

In some embodiments, the second copolymer is a random copolymer and has the structure

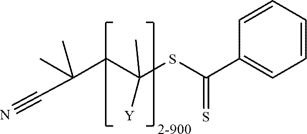

; wherein each Y is independently selected from:

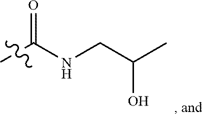

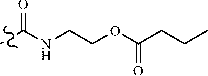

In some embodiments, the second copolymer is a block copolymer having the structure:

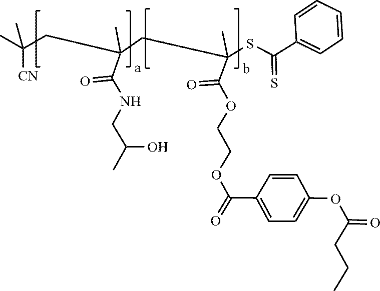

; wherein a and b are independently 1-1000. In some embodiments, the second copolymer is a random copolymer and has the structure:

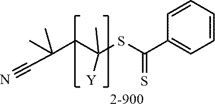

; wherein each Y is independently selected from:

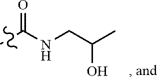

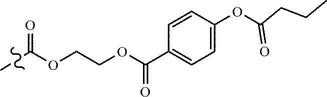

In some embodiments, the second copolymer is a block copolymer having the structure:

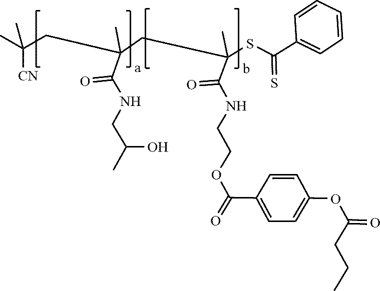

; wherein a and b are independently 1-1000. In some embodiments, the second copolymer is a random copolymer having the structure:

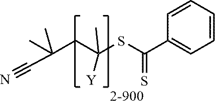

; wherein each Y is independently selected from:

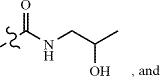

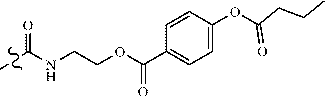

In some embodiments, provided herein are supramolecular assemblies (e.g., micelles) of the copolymers described herein. In some embodiments, the supramolecular assembly is a micelle or nanoparticle. In some embodiments, provided herein are compositions comprising two or more different types of supramolecular assemblies (e.g., micelles), for example comprising different copolymers (e.g., MAA-AMA and HPMA-AMA copolymers).

In some embodiments, provided herein are pharmaceutical compositions comprising the supramolecular assemblies (e.g., micelles) or copolymers described herein and a pharmaceutically-acceptable carrier.

In some embodiments, provided herein are foods or nutraceutical compositions comprising the supramolecular assemblies (e.g., micelles) or copolymers described herein.

In some embodiments, provided herein are methods comprising administering to a subject a pharmaceutical composition, food, or nutraceutical composition described herein. In some embodiments, the method is performed to treat or prevent a disease or condition. In some embodiments, the disease or condition is selected from the group consisting of autoimmune diseases, allergies, inflammatory conditions, infections, metabolic disorders, diseases of the central nervous system, colon cancer, diabetes, autism spectrum disorders.

In some embodiments, provided herein are methods of synthesizing or manufacturing a copolymer, supramolecular assembly (e.g., micelle), pharmaceutical composition, food, and/or nutraceutical composition described herein.

In some embodiments, provided herein is the use of a copolymer, supramolecular assembly (e.g., micelle), pharmaceutical composition, food, and/or nutraceutical composition described herein for the treatment or prevention of a disease or condition.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1¹H-NMR (500 MHz, CDCl₃) of HEMA (2)

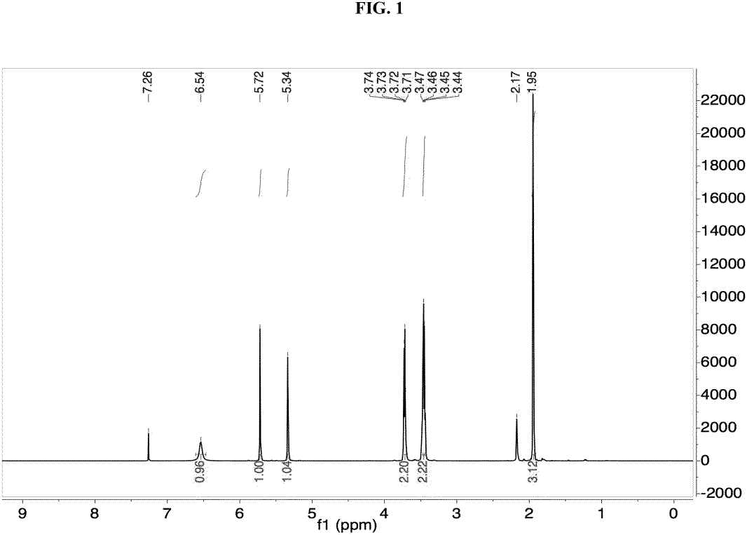

FIG. 2. ¹H-NMR (500 MHz, CDCl₃) of BMA (3)

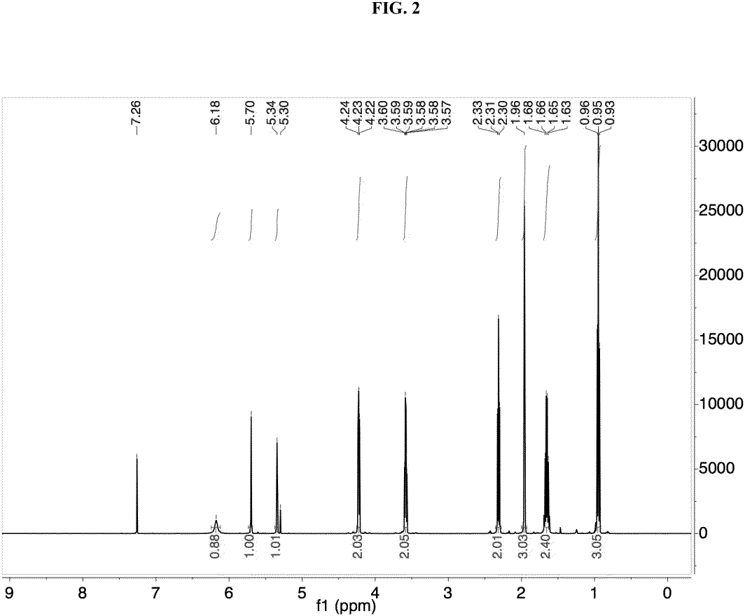

FIG. 3¹H-NMR (500 MHz, DMSO-d6) of pMAA (7)

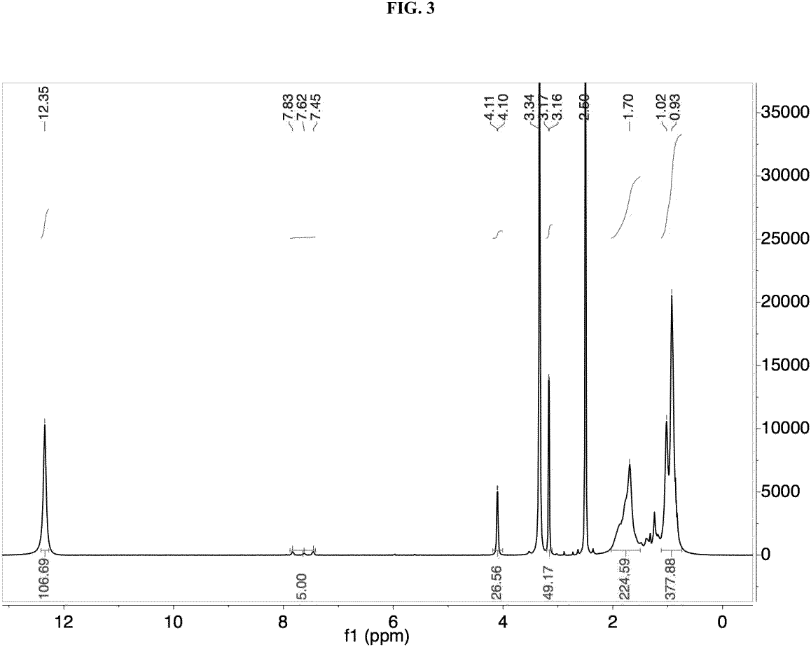

FIG. 4¹H-NMR (500 MHz, DMSO-d6) of pMAA-b-pBMA (8)

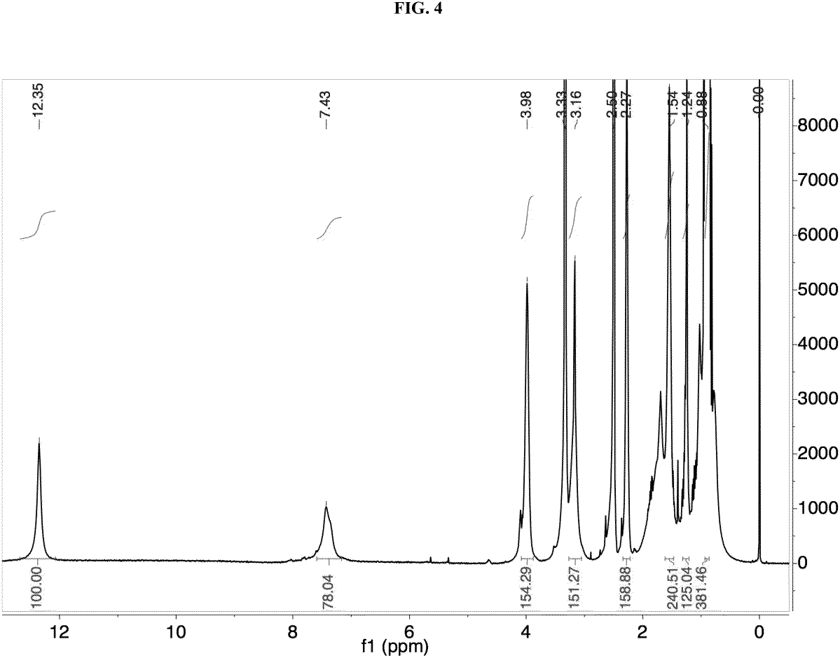

FIG. 5. Chemical composition and structural characterization of butyrate-prodrug micelles, namely NtL-ButM, consisting of the neutral block copolymer pHPMA-b-pBMA, and Neg-ButM, consisting of the anionic block copolymer pMAA-b-pBMA. A, Synthetic route of pHPMA-b-pBMA and pMAA-b-pBMA. B, (upper) The NtL-ButM contains a hydrophilic (HPMA) block as the micelle corona, while a hydrophobic (BMA) block forms the micelle core. (lower) The Neg-ButM contains a hydrophilic (MAA) block that forms a negatively charged micelle corona, and the same hydrophobic (BMA) block as NtL-ButM. C, D, Cryogenic electron microscopy (CryoEM) images show the spherical structures of micelles NtL-ButM (C) or Neg-ButM (D). E, Table summarizing the characterization of micelles NtL-ButM and Neg-ButM, including hydrodynamic diameter and zeta-potential from DLS, critical micelle concentration, radius of gyration and aggregation number from SAXS.

FIG. 6. A, Both NtL-ButM and Neg-ButM released butyrate slowly in the simulated gastric fluid over 20 days. B, Both NtL-ButM and Neg-ButM released their complete butyrate load within minutes in simulated intestinal fluid (SIF) containing high levels of the esterase pancreatin. Neither polymer released butyrate in PBS on these timescales. n = 3.

FIG. 7. The biodistribution of NtL-ButM or Neg-ButM in the gastrointestinal tract (GI) measured by In Vivo Imaging System (IVIS). Both polymers were chemically modified with azide and labeled with dye IR750. IVIS showed Neg-ButM stuck to stomach for more than 6 hours while NtL-ButM moved to cecum quickly after single oral administration to mice. Both polymers got cleared from the GI tract after 24 hours.

FIG. 8. The amount of butyrate released in the ileum, cecum, or colon contents after a single intragastric administration of NtL-ButM or Neg-ButM at 0.8 mg/g to SPF C3H/HeJ mice. Butyrate was derivatized with 3-nitrophenylhydrazine and quantified with LC-MS/MS (ileum samples) or LC-UV (cecum and colon samples). n = 9-10 mice per group. Data represent mean ± s.e.m.

FIG. 9. Tissue and cellular biodistribution of butyrate-releasing polymers. A. Representative IVIS images of lymph nodes from mice s.c. injected with fluorescently-labeled NtL-ButM or Neg-ButM in abdomen. B. Quantification of fluorescent signal from different tissues at 3 day or 7 days after s.c. injection. n = 3 mice per group. C. Percentage of fluorescent NtL-ButM or Neg-ButM positive cells among different cell subsets in inguinal LNs, small intestine draining LNs, or spleen. n = 6 mice per group per time point. Statistical anylsis was done using two-way ANOVA. ^{∗}p<0.05, ^{∗∗}p<0.005, ^{∗∗∗}p<0.0005, ^{∗∗∗∗}p<0.0001.

FIG. 10. CD40 and CD86 expression on mouse APCs in draining LNs upon s.c. LPS stimulation after s,c, administration of butyrate micelles. n = 5 mice per group. Statistical anylsis was done using two-way ANOVA. ^{∗}p<0.05, ^{∗∗}p<0.005.

FIG. 11. A. C57B/6 WT Foxp3GFP+ mice were given antibiotic water or regular water throughout the experiment, and were treated with either PBS, NtL-ButM or Neg-ButM once a week starting at 3 days after weaning for three weeks. Mice were sacrificed a week after final dose to analyze Treg populations in different tissues by flow cytometry, and the amount of butyrate by LC-MS. B. Percentage of Tregs (CD25+GFP+) among CD4+ T cells (CD45+CD3+CD4+) in different LNs and spleen of mice. C. Percentage of RoRγt+ cells among Tregs (CD45+CD3+CD4+Foxp3+CD25+) in different LNs and spleen of mice. D. Butyrate analysis in liver, spleen, serum and colon content from mice in different groups. n = 3-6 mice per group. Data represent mean ± s.e.m. Statistical analysis was done using two-way ANOVA. ^{∗}p<0.05, ^{∗∗}p<0.005, ^{∗∗∗}p<0.0005, ^{∗∗∗∗}p<0.0001.

FIGS. 12A-E. Chemical composition and structural characterization of butyrate-prodrug micelles, namely NtL-ButM, consisting of the neutral block copolymer pHPMA-b-pBMA, and Neg-ButM, consisting of the anionic block copolymer pMAA-b-pBMA. a, Synthetic route of pHPMA-b-pBMA and pMAA-b-pBMA. b, (upper) The NtL-ButM contains a hydrophilic (HPMA) block as the micelle corona, while a hydrophobic (BMA) block forms the micelle core. (lower) The Neg-ButM contains a hydrophilic (MAA) block that forms a negatively charged micelle corona, and the same hydrophobic (BMA) block as NtL-ButM. c, d, Cryogenic electron microscopy (CryoEM) images show the spherical structures of micelles NtL-ButM (c) or Neg- ButM (d). e, Table summarizing the characterization of micelles NtL-ButM and Neg-ButM, including hydrodynamic diameter and zeta-potential from DLS, critical micelle concentration, radius of gyration and aggregation number from SAXS.

FIGS. 13A-E. In vitro and in vivo butyrate release in the GI tract from NtL-ButM and Neg-ButM. a, Both NtL-ButM and Neg-ButM released butyrate slowly in the simulated gastric fluid over 20 days. b, Both NtL-ButM and Neg-ButM released their complete butyrate load within minutes in simulated intestinal fluid (SIF) containing high levels of the esterase pancreatin. Neither polymer released butyrate in PBS on these timescales. n = 3. c-e, The amount of butyrate released in the ileum, cecum, or colon contents after a single intragastric administration of NtL-ButM or Neg-ButM at 0.8 mg/g to SPF C3H/HeJ mice. Butyrate was derivatized with 3- nitrophenylhydrazine and quantified with LC-MS/MS (ileum samples) or LC-UV (cecum and colon samples). The dotted red lines represent butyrate content in untreated mice. n = 9-10 mice per group. Data represent mean ± s.e.m.

FIGS. 14A-C. NtL-ButM induced an ileal gene expression signature that is almost entirely anti- microbial peptides (AMPs). a, One week of daily dosing of 0.8 mg/g NtL-ButM to germ-free (GF) C3H/HeN mice induces a unique gene expression signature in the ileum compared to untreated and inactive polymer controls as measured by RNA sequencing of isolated intestinal epithelial cells. Top 100 significant differentially expressed genes (DEGs) at False Discovery Rate (FDR)-adjusted P<0.005 and fold change (FC) ≥ 1.5 or ≤-1.5 are shown. Annotation bars of the three groups, experiment batches (E2 and E3), and gender (female, male) are shown above the heatmap. b, Fluorescent imaging of intelectin protein in small intestine sections from control or treated mice. Blue (DAPI), red (intelectin). c, Intelectin protein is quantified by total fluorescence signal per crypt of small intestine. n = 3 PBS-treated and 4 NtL-ButM treated mice, with >15 crypts quantified per mouse. Data represent mean ± s.e.m. limma voom with precision weights was used in a. Two-sided Student’s t-test was used in c. ^{∗∗∗}P<0.001.

FIGS. 15A-D. Butyrate micelle treatment repaired intestinal barrier integrity in DSS-treated or antibiotic-treated mice. a, Mice were given 2.5% DSS in the drinking water for 7 days to induce epithelial barrier dysfunction. DSS was removed from the drinking water on days 7-10. For treatment mice were intragastrically (i.g.) dosed daily with either PBS, cyclosporin A (CsA), or ButM at different concentrations. QD: once a day, BID: twice daily at 10-12 hr intervals. On day 10, all mice received an i.g. administration of 4 kDa FITC-dextran. Fluorescence was measured in the serum 4 hr later. b, Concentration of FITC-dextran in the serum. n=8 mice per group, except for high dose ButM which had 16 mice per group. c, Mice were treated with a mixture of antibiotics, beginning at 2 wk of age, for 7 days. After weaning, mice were i.g. administered either PBS (n=10) or ButM (n=11) at 800 mg/kg twice daily for 7 days. All mice then received an i.g. administration of 4 kDa FITC-dextran. Fluorescence was measured in the serum 1.5 hr later. d, Concentration of FITC-dextran in the serum. Data in d is pooled from two independent experiments. Data represent mean ± s.e.m. Comparisons were made using one- way ANOVA with Dunnett’s post-test (c), or Student’s t-test (d). ^{∗}P<0.05, ^{∗∗}P<0.01, ^{∗∗∗}P<0.001.

FIGS. 16A-F. Butyrate micelle treatment reduced the anaphylactic response to peanut challenge. a, b, Experimental schema and dosing strategy. All of the mice were sensitized weekly by intragastric gavage of 6 mg of peanut extract (PN) plus 10 mg of the mucosal adjuvant cholera toxin. After 4 weeks of sensitization one group of mice (n=20) was challenged by i.p. administration of 1 mg of PN to confirm that the sensitization protocol induced a uniform allergic response. Fecal samples were collected before and after treatment for microbiome analysis in FIG. 6. (c). Change in core body temperature following PN challenge where core body temperature drop indicates anaphylaxis. The remaining mice were randomized into two treatment groups. One group was treated with PBS (n=32) and the other group was treated with a 1:1 mix of NtL-ButM and Neg-ButM polymers 0.4 mg/g each (n=41). QD: once a day, BID: twice daily at 10-12 hr intervals. d, Change in core body temperature following challenge with PN in PBS or ButM treated mice. The area under curve (AUC) values were compared between two groups. e,f, Serum mMCPT-1 (e) and peanut-specific IgE (f) from mice in d. Data represent mean ± s.e.m. Data in c, d and e is pooled from two independent experiments. Data analyzed using two-sided Student’s t-test. ^{∗}P<0.05, ^{∗∗∗}P<0.001.

FIGS. 17A-D. Butyrate micelles alter the fecal microbiome and promote recovery of Clostridium Cluster XIVa after antibiotic exposure. a, 16S rRNA sequencing analysis of relative abundance of bacterial taxa in fecal samples of allergic mice collected before (left) or after (right) treatment with PBS (n = 8) or ButM (n = 17) (see FIG. 5a). b, Differentially abundant taxa between mice treated with PBS or ButM after treatment as analyzed by LEfSe c, Relative abundance of Clostridium Cluster XIVa in fecal samples after treatment with PBS or ButM (from a) or d, analyzed by qPCR. For c and d, Student’s t-test with Welch’s correction was used for statistical analysis. ^{∗∗}P<0.01.

FIG. 18. 1H-NMR (500 MHz, CDCl3) of HEMA (2).

FIG. 19. 1H-NMR (500 MHz, CDCl3) of BMA (3).

FIG. 20. 1H-NMR (500 MHz, DMSO-d6) of pHPMA (5).

FIG. 21. 1H-NMR (500 MHz, DMSO-d6) of pHPMA-b-pBMA (6).

FIG. 22. 1H-NMR (500 MHz, DMSO-d6) of pMAA (7).

FIG. 23. 1H-NMR (500 MHz, DMSO-d6) of pMAA-b-pBMA (8).

FIG. 24. 1H-NMR (500 MHz, CDCl3) of N3-PEG4-MA (9).

FIG. 25. 1H-NMR (500 MHz, CDCl3) of N-hexyl methacrylamide (10).

FIG. 26. 1H-NMR (500 MHz, DMSO-d6) of control polymer pHPMA-b-pHMA.

FIG. 27. Dynamic light scattering (DLS) shows that the micelles NtL-ButM and Neg-ButM have similar hydrodynamic diameters, at sub-hundred nanometer.

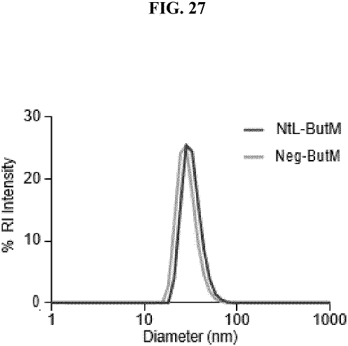

FIGS. 28A-B. Critical micelle concentrations (CMC) of NtL-ButM (left) and Neg-ButM (right) measured by pyrene fluorescent intensity of peak 1 over peak 3. The CMC was determined by the IC50 fitted by a sigmoidal curve.

FIGS. 29A-G. Small-angle X-ray scattering (SAXS) characterization of NtL-ButM and Neg-ButM micelles. a, SAXS data of NtL-ButM and Neg-ButM. Data are fitted with polydisperse core-shell model. b, Gunier plot (ln(q) vs. q2) of NtL-ButM revealed the radius of gyration of the micelle. c, Kratky plot (I q2 vs. q) of NtL-ButM revealed the spherical structure if the micelle. d, Gunier plot of Neg-ButM micelle. e, Kratky plot of Neg-ButM micelle. f, Table of fitting parameters of NtL- ButM and Neg-ButM using a polydisperse core-shell sphere model. g, Table of the mean distance between micelles d, number of micelles per unit volume N, molecular weight of the micelle Mw, and the aggregation number Nagg, calculated from the fitting parameters of a polydisperse core-shell sphere model.

FIGS. 30A-B. Derivatization of butyrate for LC-MS/MS analysis and the release of butyrate from NtL- ButM/Neg-ButM in simulated gastric/intestinal fluids. a, Derivatization reaction of butyrate with 3-nitrophenylhydrazine (NPH) to generate UV active butyrate-NPH. b, The multiple reaction monitoring (MRM) of 222 → 137 was used to quantify butyrate-NPH in LC-MS/MS.

FIGS. 31A-D. Stability of pHPMA-b-pBMA polymer in vitro or in vivo. a, Gel permeation chromatography (GPC) elution profiles (measured by differential refractive index (dRI) over time) of polymers collected from pooled fecal samples of two mice treated with NtL-ButM at 4-6 hr (red) or 6-8 hr (blue) post-gavage. Black curve: polymer control. b, The table of molecular weight of digested polymer measured by GPC, including the number averaged molecular weight (Mn) and weight averaged molecular weight (Mw), with their polydispersity index (PDI) and the Mn loss compared to undigested polymer control. c, d, The Mn loss of pHPMA-b-pBMA polymer in 125 mM NaOH solution over 7 days (c), or the percentage of butyrate released from the polymer (d), measured by GPC, n=3, Data represent mean ± s.e.m.

FIGS. 32A-B. The biodistribution of NtL-ButM or Neg-ButM in the gastrointestinal (GI) tract a, and other major organs and serum b, measured by in vivo imaging system (IVIS). Both polymers were chemically modified with azide and labeled with dye IR750. After a single oral administration of NtL-ButM or Neg-ButM (one mouse per time point per treatment group), IVIS showed Neg-ButM retained in the stomach for more than 6 hr. while NtL-ButM moved to the cecum quickly after a single intragastric administration to mice. Both polymers were cleared from the GI tract after 24 hr., and there was no absorption of either butyrate micelle into the systemic circulation. Mesenteric LNs (d, duodenum-draining; j, jejunum-draining; I, ileum- draining; c, colon-draining).

FIG. 33. Differentially expressed genes (DEGs) in the ileum of GF mice that were treated with daily 0.8 mg/g NtL-ButM for one week, compared to untreated and inactive polymer controls as measured by RNA sequencing of isolated ileal epithelial cells (see FIG. 3). The unit of the value is TMM-normalized and log2-transformed read counts.

FIGS. 34A-F. Butyrate micelle treatment reduced the anaphylactic response to peanut challenge in a dose-dependent manner. a, b, Experimental schema and the dosing strategy. All of the mice were sensitized weekly by i.g. gavage of 6 mg of PN plus 10 □g of the mucosal adjuvant cholera toxin. c, A uniform allergic response was confirmed by challenging one group of mice (n=7) with 1 mg of PN i.p. and measuring reduction in core body temperature as an indication of anaphylaxis. QD: once a day. d-f, The rest of mice were randomized into three treatment groups and received either PBS (n=8), ButM at 0.4 mg/g (half dose) (n=11), or ButM at 0.8 mg/g (full dose) (n=9) twice daily. d, Change in core body temperature following challenge with peanut extract. The area under curve (AUC) was compared among groups. e, f, Serum mMCPT-1 (e) and peanut-specific IgE (f) from mice in d. Data represent mean ± s.e.m. Data analyzed using one-way ANOVA with Dunnett’s post-test. ^{∗}P<0.05.

FIGS. 35A-F. Butyrate micelle treatment in conjunction with low dose PN reduced the anaphylactic response to peanut challenge. a, b, Experimental schema and the dosing strategy. All of the mice were sensitized weekly by i.g. gavage of 6 mg of PN plus 10 □g of the mucosal adjuvant cholera toxin. c, A uniform allergic response was confirmed by challenging one group of mice (n=46) with 1 mg of PN i.p. and measuring reduction in core body temperature as an indication of anaphylaxis. QD: once a day. d, The rest of mice were randomized into two treatment groups. One group was treated with low dose PB2□ (200 µg, blue) once daily (n=26), and another group was treated with low dose PB2□ 200 µg once daily plus ButM at 0.8 mg/g twice daily (n=29). d, Change in core body temperature following challenge with peanut extract in low dose PN or low dose PN + ButM treated mice. The area under curve (AUC) was compared between two groups. e, f, Serum mMCPT-1 (e) and peanut-specific IgE (f) from mice in d. Data represent mean ± s.e.m. Data analyzed using Student’s t-test. ^{∗∗}P<0.01. ns, not significant.

FIGS. 36A-B. Differentially abundant taxa within each treatment group before and after two-week treatment with PBS (a) or ButM (b) as analyzed by LEfSe from FIG. 6.

FIGS. 37A-B. NtL-ButM showed no serological toxicity in mice. SPF C3H/HeJ mice were treated with PBS, sodium butyrate (NaBut), or NtL-ButM daily for 6 wk. Mouse serum samples were measured on a chemistry analyzer for six toxicity markers every week. Results of alanine aminotransferase (ALT) level on week 6 are shown in a, as an example. b, None of the markers showed a significant difference between NtL-ButM group and PBS group. Data represent mean ± s.e.m. Comparisons were made using one-way ANOVA with Dunnett’s post-test. n.s., not significant.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a block copolymer” is a reference to one or more block copolymers and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “short-chain fatty acid” (“SCFA”) refers to a carboxylic acid attached to an aliphatic chain, which is either saturated or unsaturated, the aliphatic chain being 12 carbons or less in length.

As used herein the term “fatty acid derivative” (specifically “SCFA derivative”) refers to a small molecular compounds that are obtained by making simple modifications (e.g., amidation, methylation, halogenation, etc.) to fatty acid molecules (e.g., SCFA molecules). For example, butyramide, D- or L-amino-n-butyric acid, alpha- or beta-amino-n-butyric acid, arginine butyrate, butyrin, phenyl butyrates (e.g., 4-, 3-, 2-), dimethylbutyrate, 4-halobutyrates (e.g., fluoro-, chloro-, bromo-, iodo-), 3-halobutyrates (e.g., fluoro-, chloro-, bromo-, iodo-), 2-halobutyrates (e.g., fluoro-, chloro-, bromo-, iodo-), oxybutyrate, and methylbutyrates are exemplary butyrate derivatives. Other butyrate derivatives and similar derivatives of other SCFAs are within the scope of the SCFA derivatives described herein.

As used herein, the term “copolymer” refers to a polymer formed from two or more different monomer subunits. Exemplary copolymers include alternating copolymers, random copolymers, block copolymers, etc.

As used herein, the term “block copolymer” refers to copolymers wherein the repeating subunits are polymeric blocks, i.e. a polymer of polymers. In a copolymer of blocks A and B, A and B each represent polymeric entities themselves, obtained by the polymerization of monomers. Exemplary configurations of such block copolymers include branched, star, di-block, tri-block and so on.

As used herein, the term “supramolecular” (e.g., “supramolecular assembly” (e.g., “micelle”)) refers to the non-covalent interactions between molecules and/or solution (e.g., polymers, macromolecules, etc.) and the multicomponent assemblies, complexes, systems, and/or fibers that form as a result. In some embodiments, a micelle is a supramolecular assembly resulting from non-covalent interactions between, for example, copolymers in a colloidal solution.

As used herein, the term “dysbiosis” refers to a reduction in microbial diversity, including a rise in pathogenic bacteria and/or the loss of beneficial bacteria such as Bacteroides strains, Enterococcus, Coprobacter, and Clostridium Cluster XIVa bacrteria, as well as other butyrate-producing bacteria such as Firmicutes.

As used herein, the term “abundance,” when used in reference to bacteria, refers to the amount of a type of bacteria present. The term “relative abundance,” when used in reference to bacteria, refers to the amount of the type of bacteria present as compared to the overall amount of bacteria present.

As used herein, the term “pharmaceutically acceptable carrier” refers to non-toxic solid, semisolid, or liquid filler, diluent, encapsulating material, formulation auxiliary, excipient, or carrier conventional in the art for use with a therapeutic agent for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. A “pharmaceutical composition” typically comprises at least one active agent (e.g., the copolymers described herein) and a pharmaceutically acceptable carrier.

As used herein, the term “effective amount” refers to the amount of a composition (e.g., pharmaceutical composition) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., pharmaceutical compositions of the present invention) to a subject or in vivo, in vitro, or ex vivo cells, tissues, and organs. Exemplary routes of administration to the human body can be through the eyes (e.g., intraocularly, intravitreally, periocularly, ophthalmic, etc.), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the terms “co-administration” and “co-administer” refer to the administration of at least two agent(s) or therapies to a subject. In some embodiments, the co-administration of two or more agents or therapies is concurrent (e.g., in the same or separate formulations). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “nanoparticles” refers to particles having mean dimensions (e.g., diameter, width, length, etc.) of less than 1 µm (e.g., <500 nm (“sub-500-nm nanoparticles”), <100 nm (“sub-100-nm nanoparticles”), <50 nm (“sub-50-nm nanoparticles”).

As used herein, the term “biocompatible” refers to materials, compounds, or compositions means that do not cause or elicit significant adverse effects when administered to a subject. Examples of possible adverse effects that limit biocompatibility include, but are not limited to, excessive inflammation, excessive or adverse immune response, and toxicity.

As used herein, the term “biostable” refers to compositions or materials that do not readily break-down or degrade in a physiological or similar aqueous environment. Conversely, the term “biodegradeable” refers herein to compositions or materials that readily decompose (e.g., depolymerize, hydrolyze, are enzymatically degraded, disassociate, etc.) in a physiological or other environment.

As used herein, the term “substituted” refers to a group (e.g., alkyl, etc.) that is modified with one or more additional group(s). Non-limiting examples of substituents include, for example: halogen, hydroxy, oxo (═O), thioxo (═S), cyano (—CN), nitro (—NO₂), imino (═N—H), oximo (═N—OH), hydrazino (═N—NH2), NH₂)—R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, each of which may be optionally substituted by halogen, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})2, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a} , —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), —R^{b}—S(O)ₜN(R^{a})₂ (where t is 1 or 2), carbocycle and heterocycle; wherein each R^{a} is independently selected from hydrogen, alkyl, alkenyl, alkynyl, carbocycle and heterocycle, wherein each R^{a}, valence permitting, may be optionally substituted with halogen, oxo (═O), thioxo (═S), cyano (—CN), nitro (—NO₂), imino (═N—H), oximo (═N—OH), hydrazine (═N—NH2), —R^{b}—OR^{a}, —R^{b}—OC(O)—R^{a}, —R^{b}—OC(O)—OR^{a}, —R^{b}—OC(O)—N(R^{a})2, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a} , —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain. Substituent groups may be selected from, but are not limited to: alkyl, alkenyl, alkynyl, cycloalkyl, aryl, heteroaryl, heterocycloalkyl, hydroxyl, alkoxy, mercaptyl, cyano, halo, carbonyl, thiocarbonyl, isocyanato, thiocyanato, isothiocyanato, nitro, perhaloalkyl, perfluoroalkyl, and amino, including mono- and di-substituted amino groups, and the protected derivatives thereof. A “substituted alkyl” encompasses alkynes and alkenes, in addition to alkanes displaying substituent moieties.

As used herein, the term “pseudo-random” refers to sequences or structures generated by processes in which no steps or measures have been taken to control the order of addition of monomers or components.

As used herein, the term “display” refers to the presentation of solvent-exposed functional group by a molecule, monomer, polymer, nanostructure or other chemical entity.

### DETAILED DESCRIPTION OF THE INVENTION

Provided herein are polymer materials that find use in, for example, delivery of short-chain fatty acids. In particular, polymers are provided that form stable nanoscale structures (e.g., micelles) and release their payload, for example, by cleavage of a covalent bond (e.g., via hydrolysis or enzymatic cleavage). The polymers are useful, for example, for delivery of payloads (e.g., SCFAs) to the intestine for applications in health and treatment of disease, and have broad applicability in diseases linked to changes in the human microbiota including inflammatory, autoimmune, allergic, metabolic, and central nervous system diseases, among others. In some embodiments, provided herein are prodrug polymeric micelles that find use in the delivery of short-chain fatty acids to the intestine for the promotion of gut health, establishment of healthy microbiota, treatment of immune and/or inflammatory conditions, such as inflammatory bowel disease and food allergies.

Experiments were conducted during development of embodiments herein to develop block copolymers that can water- suspensible micelles carrying a high content of butyrate in their core. These polymer formulationsmask the smell and taste of butyrate and act as carriers to release the active ingredient (e.g., SCFA (e.g., butyrate)) over time as the micelles transit the GI tract. Experiments conducted during development of embodiments herein show that these butyrate-conjugated polymer formulations up-regulate AMP gene expression in the ileal epithelium and modulate barrier integrity in antibiotic-treated mice and in mice treated with dextran sodium sulfate (DSS), a chemical perturbant that induces epithelial barrier dysfunction. Intragastric administration of our butyrate-prodrug micelles ameliorates an anaphylactic response to peanut challenge in a mousemodel of peanut allergy and increases the abundance of bacteria in a cluster (Clostridium ClusterXIVa) known to contain butyrate-producing taxa.

The prevalence of food allergy has increased dramatically over the past 20 years, particularly in developed countries (Refs. B7, B46; incorporated by reference in their entireties). Lifestyle changes such as reduced consumption of dietary fiber, increased antibiotic use (including in the food chain), and sanitation, have altered populations of commensal microbes. These alterations lead to several negative health effects, including impairment of intestinal barrier function. Modulating the gut microbiome to redirect immunity has become a substantial effort in both academia and industry. However, this has proven difficult: getting selected organisms, especially obligate anaerobes, to colonize the gut is far from straightforward.

Provided herein are polymeric nano-scale systems to deliver SCFAs (e.g., butyrate) to localized regionsalong the GI tract. The system was based on polymeric micelles formed by block copolymers, inwhich SCFAs (e.g., butyrate) is conjugated to the hydrophobic block by an ester bond and can be hydrolyzed by esterases in the GI tract for local release. The linked butyrate moieties drive hydrophobicity inthat block and, as release occurs, the remainder of the construct (an inert, water-soluble polymer) continues to transit through the lower GI tract until it is excreted. The butyrate-containing block, when forming the core of micelles, was resistant to the acidic environment found in the stomach,which might prevent a burst release there before the micelle’s transit into the intestine. The two butyrate-prodrug micelles, NtL-ButM and Neg-ButM, share similar structures but have corona charges of neutral and negative, respectively. This results in their distinct biodistribution in the lower GI tract, where they can release butyrate in the presence of enzymes. Experiments were conducted during development of embodiments herein to treat a mouse model of peanut allergy and to repair intestinal barrier dysfunction, using both theneutral and negatively charged micelles to deliver butyrate along the distal gut and showed successful preservation of barrier function and protection from severe anaphylactic responses with a short-term treatment. The butyrate micelles were not absorbed in the small intestine and could act locally by inducing a gene expression signature that is comprised almost entirely of AMPs. These AMPs, mainly expressed by specialized Paneth cells in the ileum, are essential formaintaining the balance of the ileal microbiome (Ref. B15; incorporated by reference in its entirety). In the mouse model of peanut allergy, where the mice were previously exposed to vancomycin to induce dysbiosis, ButM treatment favorably increased the relative abundance of protective bacteria, such as Clostridium Cluster XIVa. Bacteria in Clostridium Cluster XIVa are known to induce local Tregs inpreclinical models and may be critical to the success of fecal microbiota transplant for treatment of colitis (Refs. B43, B47; incorporated by reference in their entireties).

Investigations of the therapeutic potential of butyrate in animal models have supplemented butyrate in the drinking water or diet at a high dose for three or more weeks (Refs. B16-B18, B23-B25: incorporated by reference in their entireties). Such dosing to achieve therapeutic effects from sodium butyrate is challenging for clinical translation, due to the uncontrolled dosing regimen, difficulties to replicate in humans, and the unpleasant odor andtaste of butyrate as a sodium salt. The formulation use is the experiments conducted during development of embodiments herein incorporated butyrate in the polymeric micelles at a high load (28 wt%) and is able to deliver and release most of the butyrate in the lower GI tract, in a manner that masks butyrate’s taste and smell. A daily dose of 800 mg/kg of total ButM was used to treat peanut allergic mice for two weeks. This can be translatedto ~65 mg/kg of total ButM (or equivalent butyrate dose of 18.2 mg/kg) human dose given the differences in body surface area between rodents and the human (Ref. B48; incorporated by reference in its entirety). This butyrate dose in ButM micelles is comparable to other butyrate dosage forms that has been tested clinically (Refs, B49-B51; incorporated by reference in their entireties), however, through the local targeting and sustained release in the lower GI tract, we expect our ButM formulation to achieve higher therapeutic potential in food allergies and beyond.

The present approaches are not antigen-specific, and therefore can be readily extended to other food allergens, such as other nuts, milk, egg, soy and shellfish. Moreover, the platform can also be easily adapted to deliver other SCFAs or other microbiome-derived metabolites in a single form or in combination,providing a more controlled and accessible way to achieve potential therapeutic efficacy.

In a first aspect, provided herein are copolymers (and micelles thereof) comprising a methacrylic acid (MAA) monomer (or a block thereof) and a prodrug-containing monomer (e.g., with a SCFA sidechain).

In a second aspect, provided herein are compositions comprising a first copolymer assembly (e.g., first micelle) comprising a first copolymer comprising a methacrylic acid (MAA) monomer (or a block thereof) and a prodrug-containing monomer (e.g., with a SCFA sidechain), and a second copolymer assembly (e.g., second micelle) comprising a second copolymer comprising a N-(2-hydroxyethyl) methacrylamide (HPMA) monomer (or a block thereof) and a prodrug-containing monomer (e.g., with a SCFA sidechain).

In a third aspect, provided herein are pharmaceutical or nutraceutical compositions comprising the copolymers herein and noncovalent assemblies (e.g.. micelles) thereof (e.g., (1) MAA/prodrug copolymers and micelles, (2) micelles of MAA/prodrug copolymers and micelles of HPMA/prodrug copolymers, etc.) and methods of administering such pharmaceutical or nutraceutical compositions, for example for the treatment or prevention of inflammatory, autoimmune, allergic, metabolic, and central nervous system diseases or for the regulation of microbiota levels.

In a fourth aspect, provided herein are methods for detecting/monitoring metabolomic and/or microbiomic biomarkers in a subject and administering the compositions described herein to correct or regulate the levels thereof (e.g., for the treatment or prevention of inflammatory, autoimmune, allergic, metabolic, central nervous system diseases, etc.).

In some embodiments, provided herein are copolymers (e.g., block or random) comprising methacrylic acid (MAA) monomers (or a block thereof) and a prodrug monomer (e.g., comprising a SCFA sidechain). In some embodiments, methods are provided for the assembly of these copolymers into nanoparticles, micelles, or other delivery systems. In some embodiments, methods are provided for the administration of the copolymers, and delivery systems comprising such copolymers, for the treatment or prevention of various diseases and conditions. In particular, polymers are functionalized to deliver a pharmaceutically-relevant small molecule moiety (e.g., SCFA) relevant for treating human disease with a covalent bond that is broken (e.g., by hydrolysis or enzyme activity).

In some embodiments, copolymers (e.g., block or random) comprising methacrylic acid (MAA) monomers (or a block thereof) and a prodrug monomer (e.g., comprising a SCFA sidechain), or assemblies thereof (e.g., micelles thereof) are provided (e.g., as part of a composition or system) with copolymers (e.g., block or random) comprising N-(2-hydroxyethyl) methacrylamide (HPMA) monomers (or a block thereof) and the prodrug monomer (e.g., comprising a SCFA sidechain), or assemblies thereof (e.g., micelles thereof)

In some embodiments, copolymers herein are obtained using reversible addition-fragmentation chain-transfer (“RAFT”) polymerization of an appropriate monomer with an initiator.

In some embodiments, a free terminus of the polymer may be one of a number of chemical groups, including but not limited to hydroxyl, methoxy, benzyl, cyano, thiol, amine, maleimide, halogen, polymer chain transfer agents, protecting groups, drug, biomolecule, or tissue targeting moiety. Some or all polymers display a pharmaceutically-relevant small molecule covalently attached to a hydroxyethyl functional group. A preferred embodiment of the pharmaceutically-relevant small molecule is short- and medium-chain fatty acids (“SCFA”s) and their derivatives containing up to 12 carbon atoms in the chain, for example, between 3 and 10 carbon atoms in the chain. The chain may be linear or branched. Example SCFAs include, but are not limited to, acetate, propionate, iso-propionate, butyrate, iso-butyrate, and other SCFAs described herein, as well as derivatives thereof. A free SCFA terminus may be one of a number of chemical groups including but not limited to methyl, hydroxyl, methoxy, thiol, amine, N-alkyl amine, and others.

In some embodiments, an MAA or HPMA copolymer is a copolymer (e.g., random copolymer) of MAA or HPMA monomers and N-hydroxyethyl methacrylate monomers. A free MAA or HPMA terminus may be one of a number of chemical groups, including but not limited to hydroxyl, cyano, benzyl, methoxy, thiol, amine, maleimide, halogen, polymer chain transfer agents, protecting groups, drug, biomolecule, or tissue targeting moiety. Some or all N-hydroxyethyl methacrylate monomers display a pharmaceutically-relevant small molecule covalently attached to the hydroxyethyl functional group. A preferred embodiment of the pharmaceutically-relevant small molecule is short- and medium-chain fatty acids (“SCFA”s) and their derivatives containing, for example, between 3 and 12 (e.g., 3, 4, 5, 6, 7, 8, 9, 10, 11, 12, or ranges therebetween) carbon atoms in the chain. The chain may be linear or branched. Example SCFAs include, but are not limited to, acetate, propionate, iso-propionate, butyrate, iso-butyrate, and other SCFAs described herein, as well as derivatives thereof. A free SCFA terminus may be one of a number of chemical groups including but not limited to methyl, hydroxyl, methoxy, thiol, amine, N-alkyl amine, and others.

Blocks may vary in molecular weight and therefore size, the adjustment of which alters the ratio of inert, unfunctionalized, pharmaceutically inactive material and active, functionalized pharmaceutically-active material. Some embodiments are a linear MAA or HPMA block copolymer whose relative block sizes are between 0.25 and 3.5 (e.g., 0.25, 0.3, 0.35, 0.4, 0.45, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, and ranges therebetween (e.g., 0.7 -1.8)). Other embodiments are a linear MAA block copolymer whose relative block sizes are between 0.25 and 3.5 (e.g., 0.25, 0.3, 0.35, 0.4, 0.45, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, and ranges therebetween (e.g., 0.7 - 1.8)). A “relative block size” of 0.25 - 3.5 means that for every 1 mole of MAA or HPMA weight, the N-oxyethyl methacrylamide block (or a SCFA functionalized derivative) is 0.25 mole - 3.5 mole). In some embodiments, block copolymers described herein form nanoparticles or micelles of diameter 10-1000 nm (e.g., 10, 20, 50, 100, 200, 500, 1000 nm, or ranges therebetween (e.g., 50-500 nm) when dispersed (e.g., in a liquid). The nanoparticles or micelles thus formed can then be isolated as a solid (e.g., in a powder, by lyophilization, etc.) with or without stabilizers (e.g., surfactants).

The MAA or HPMA block may be present at a molecular weight of between 3000 and 50,000 Da (e.g., 3000, 4000, 5000 Da, 6000 Da, 7000 Da, 8000 Da, 9000 Da, 10000 Da, 11000 Da, 12000 Da, 13000 Da, 14000 Da, 15000 Da, 20000 Da, 25000 Da, 30000 Da, 35000 Da, 40000 Da, 45000 Da, 50000 Da, or ranges therebetween (e.g., 9000-14000 Da, 14000-30000)).

In some embodiments, provided herein are compositions comprising a copolymer of (i) a monomer comprising methacrylic acid (MAA) and (ii) a monomer of formula (I):

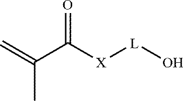

, wherein X is O, NH, or S; wherein L is a linker selected from an alkyl chain, an heteroalkyl chain, a substituted alkyl chain, or a substituted heteroalkyl chain; wherein the copolymer displays one or more short-chain fatty acid (SCFA) moieties.

In some embodiments, provided herein are compositions comprising a copolymer of (i) a monomer comprising methacrylic acid (MAA) and (ii) a monomer of formula (II):

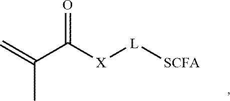

wherein X is O, NH, or S; wherein L is a linker selected from an alkyl chain, an heteroalkyl chain, a substituted alkyl chain, or a substituted heteroalkyl chain; and wherein SCFA is a short-chain fatty acid.

In some embodiments, L of formula (I) or formula (II) is (CH₂)ₙ, wherein n is 1-16 (e.g., 1, 2, 3, 4, 5, 6, 7, 8, 9, 10, 11, 12, 13, 14, 15, 16, or ranges therebetween). In some embodiments, L is (CH₂)ₙO(CO)-benzene. In some embodiments, the SCFA is covalently attached to the monomer of formula (I). In some embodiments, the SCFA attached to the monomer of formula (I) comprises formula (II):

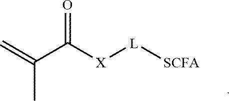

In some embodiments, the SCFA attached to the monomer of formula (I) or formula (II) comprises formula (III):

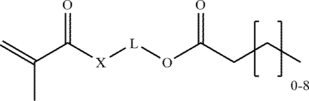

. In some embodiments, the SCFA is selected from the group consisting of acetic acid, propionic acid, isopropionic acid, butyric acid, isobutyric acid, valeric acid, isovaleric acid, caproic acid, caprylic acid, capric acid, lauric acid, branched versions thereof, and derivatives thereof. In some embodiments, the SCFA is butyric acid.

In some embodiments, the copolymer is a block copolymer comprising an MAA block and a block of formula (I) or formula (II). In some embodiments, the block copolymer

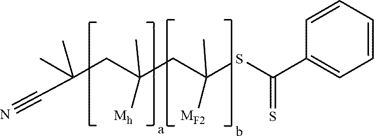

comprises the formula (IV) ; wherein Mₕ comprises MAA, M_{F2} is the side chain of the monomer of Formula (II):

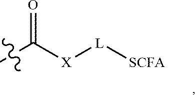

wherein a is 1-1000 (e.g., 10, 20, 30, 40, 50, 60, 70, 80, 90, 100, 125, 133, 150, 175, 200, 250, 300, 400, 500, 600, 700, 800, 900, 1000, or ranges therebetween) and b is 1-1000 (e.g., 10, 20, 30, 40, 50, 60, 70, 80, 90, 100, 125, 133, 150, 175, 200, 250, 300, 400, 500, 600, 700, 800, 900, 1000, or ranges therebetween).

In some embodiments, the copolymer is a random copolymer. In some embodiments, the random copolymer comprises formula (V):

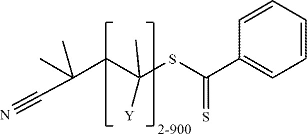

; wherein each Y is independently selected from the side chain of a polymer formed from formula (II):

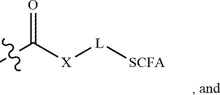

the side chain of MAA:

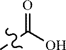

. In some embodiments, there are 2, 5, 10, 20, 30, 40, 50, 75, 100, 125, 150, 175, 200, 250, 300, 350, 400, 450, 500, 550, 600, 650, 700, 750, 800, 850, 900, or ranges therebetween of the repeated Y-displaying groups.

In some embodiments, the monomer of formula (II) comprises N-butanoyloxyalkyl methacrylamide. In some embodiments, the N-butanoyloxyalkyl methacrylamide monomer is 2-butanoyloxyethyl methacrylamide. In some embodiments, the copolymer is a block copolymer and comprises formula (VI):

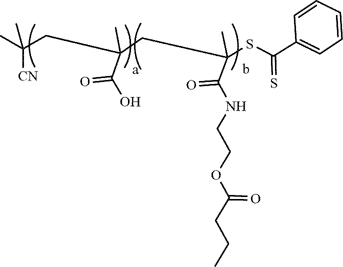

; wherein a and b are independently 1-1000 (e.g., 10, 20, 30, 40, 50, 60, 70, 80, 90, 100, 125, 133, 150, 175, 200, 250, 300, 400, 500, 600, 700, 800, 900, 1000, or ranges therebetween). In some embodiments, the copolymer is a random copolymer and comprises formula (V):

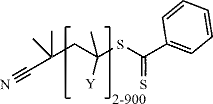

; wherein each Y is independently selected from (i) the side chain of MAA:

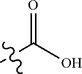

, and (ii) the side chain of poly(2-butanoyloxyethyl methacrylamide). In some embodiments, there are 2, 5, 10, 20, 30, 40, 50, 75, 100, 125, 150, 175 200, 250, 300, 350, 400, 450, 500, 550, 600, 650, 700, 750, 800, 850, 900, or ranges therebetween of the repeated Y-displaying groups.

In some embodiments, the monomer of formula (II) comprises an N-butanoyloxyalkyl methacrylate. In some embodiments, the N-butanoyloxyalkyl methacrylate monomer is an 2-butanoyloxyethyl methacrylate. In some embodiments, the copolymer is a block copolymer and comprises formula (VII):

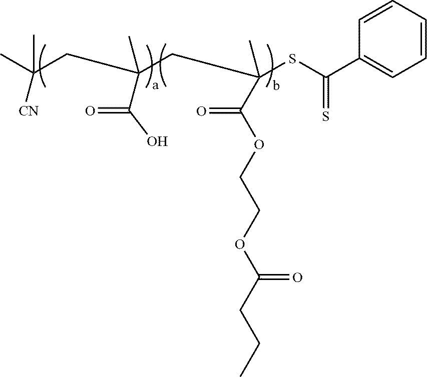

; wherein a and b are independently 1-1000 (e.g., 10, 20, 30, 40, 50, 60, 70, 80, 90, 100, 125, 133, 150, 175, 200, 250, 300, 400, 500, 600, 700, 800, 900, 1000, or ranges therebetween). In some embodiments, the copolymer is a random copolymer and comprises formula (V):

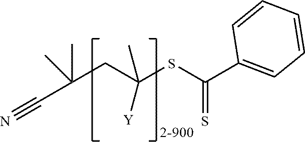

; wherein each Y is independently selected from (i) the side chain of MAA:

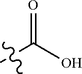

, and (ii) the side chain of poly(2-butanoyloxyethyl methacrylate). In some embodiments, there are 2, 5, 10, 20, 30, 40, 50, 75, 100, 125, 150, 175, 200, 250, 300, 350, 400, 450, 500, 550, 600, 650, 700, 750, 800, 850, 900, or ranges therebetween of the repeated Y-displaying groups.

In some embodiments, the monomer of formula (II) comprises an N-(4-butanoyloxybenzoyloxy)alkyl methacrylate. In some embodiments, the N-(4-butanoyloxybenzoyloxy)alkyl methacrylate monomer is 2-(4-butanoyloxybenzoyloxy)ethyl methacrylate. In some embodiments, the copolymer is a block copolymer and comprises formula (VIII):

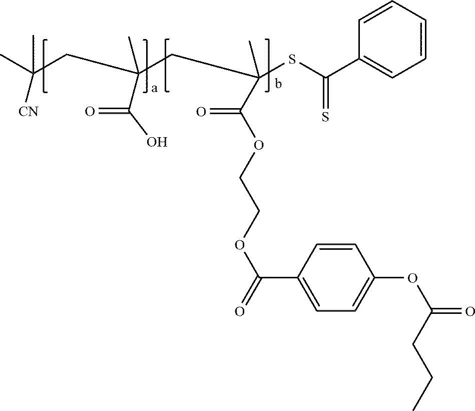

; wherein a and b are independently 1-1000 (e.g., 10, 20, 30, 40, 50, 60, 70, 80, 90, 100, 125, 133, 150, 175, 200, 250, 300, 400, 500, 600, 700, 800, 900, 1000, or ranges therebetween). In some embodiments, the copolymer is a random copolymer and comprises formula (V):

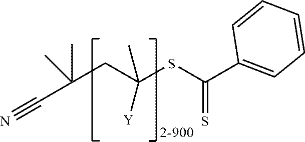

; wherein each Y is independently selected from (i) the side chain of MAA:

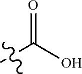

and (ii) the side chain of poly(2-(4-butanoyloxybenzoyloxy)ethyl methacrylate):

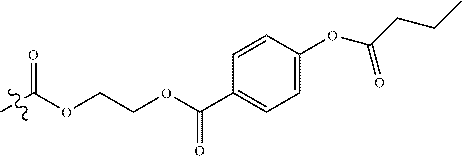

. In some embodiments, there are 2, 5, 10, 20, 30, 40, 50, 75, 100, 125, 150, 175, 200, 250, 300, 350, 400, 450, 500, 550, 600, 650, 700, 750, 800, 850, 900, or ranges therebetween of the repeated Y-displaying groups.

In some embodiments, the monomer of formula (II) comprises an N-(4-butanoyloxybenzoyloxy)alkyl methacrylamide. In some embodiments, the N-(4-butanoyloxybenzoyloxy)alkyl methacrylamide monomer is 2-(4-butanoyloxybenzoyloxy)ethyl methacrylamide. In some embodiments, the copolymer is a block copolymer and comprises formula (IX):

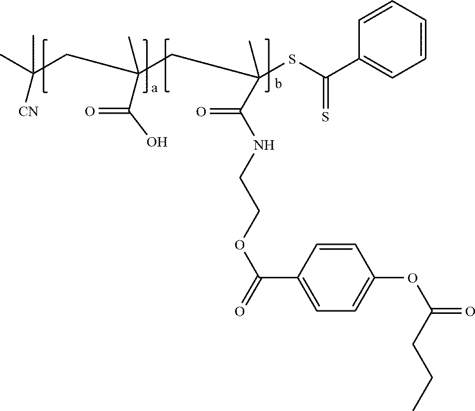

; wherein a and b are independently 1-1000 (e.g., 10, 20, 30, 40, 50, 60, 70, 80, 90, 100, 125, 133, 150, 175, 200, 250, 300, 400, 500, 600, 700, 800, 900, 1000, or ranges therebetween). In some embodiments, the copolymer is a random copolymer and comprises formula (V):

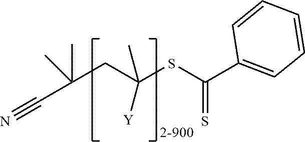

; wherein each Y is independently selected from (i) the side chain of MAA:

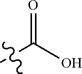

, and (ii) the side chain of poly(2-(4-butanoyloxybenzoyloxy)ethyl methacrylamide):

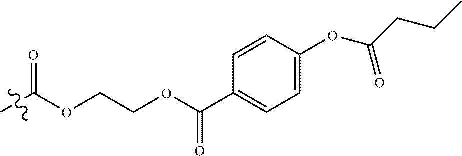

. In some embodiments, there are 2, 5, 10, 20, 30, 40, 50, 75, 100, 125, 150, 175, 200, 250, 300, 350, 400, 450, 500, 550, 600, 650, 700, 750, 800, 850, 900, or ranges therebetween of the repeated Y-displaying groups.

In certain embodiments, the copolymer compositions herein are administered in the form of a pharmaceutical composition, a dietary supplement, or a food or beverage. When the compositions herein are used as a food or beverage, the food or beverage can be, e.g., a health food, a functional food, a food for a specified health use, a dietary supplement, or a food for patients. The composition may be administered once or more than once. If administered more than once, it can be administered on a regular basis (e.g., two times per day, once a day, once every two days, once a week, once a month, once a year) or on as needed, or irregular basis. The frequency of administration of the composition can be determined empirically by those skilled in the art.

Release of the pharmaceutically-active small molecule (e.g., SCFA) is a necessarily important aspect of the copolymer performance for material processing or downstream biological applications. In some embodiments, the pharmaceutically-active small molecule may be cleaved from the polymer backbone under suitable biological conditions, including hydrolysis (e.g., at certain pH) and enzyme activity (e.g., an esterase). In this regard, the copolymer may be termed a prodrug. In various embodiments, the pharmaceutical composition includes about 10-80% (e.g., 10%, 11%, 12%, 13%, 14%, 15%, 20%, 25%, 30%, 35%, 40%, 45%, 50%, 60%, 70%, 80%, or ranges therebetween) of pharmaceutically-active small molecule, e.g., a SCFA or derivative thereof, by weight. Those skilled in the art of clinical pharmacology can readily arrive at dosing amounts using routine experimentation.

Release of the pharmaceutically-active small molecule necessarily has a therapeutic effect recapitulating the therapeutic effects of SCFAs, including targeting the barrier function of the intestine and the mucus layer of the gut and all diseases in which SCFAs have been implicated to have a therapeutic benefit, including increasing mucus layer thickness or barrier function are implicated may be treated. In some embodiments, the human diseases that are treatable include, but are not limited to, rheumatoid arthritis, celiac disease and other autoimmune diseases, food allergies of all types, eosinophilic esophagitis, allergic rhinitis, allergic asthma, pet allergies, drug allergies, and other allergic and atopic diseases, inflammatory bowel disease, ulcerative colitis, Crohn’s dieases, and additional inflammatory conditions, infectious diseases, metabolic disorders, multiple sclerosis, Alzheimer’s disease, Parkinson’s disease, dementia, and other diseases of the central nervous system, thalassemia and other blood disorders, colorectal cancer, diarrhea and related diseases effecting gut motility, Type I diabetes, and autism spectrum disorders, among others. This list is not exhaustive, and those skilled in the art can readily treat additional indications that have been shown to have therapeutic effect of SCFAs.

Pharmaceutical preparations can be formulated from the composition of the invention by drug formulation methods known to those skilled in the art. Formulations are prepared using a pharmaceutically acceptable “carrier” composed of materials that are considered safe and effective, without causing undesirable biological side effects or unwanted interactions. Suitable carriers include, but are not limited to, saline, buffered saline, dextrose, water, glycerol, ethanol, and combinations thereof. The composition can be adapted for the mode of administration and can be in the form of, e.g., a pill, tablet, capsule, spray, powder, or liquid. In some embodiments, the pharmaceutical composition contains one or more pharmaceutically acceptable additives suitable for the selected route and mode of administration, such as coatings, fillers, binders, lubricant, disintegrants, stabilizers, or surfactants. These compositions may be administered by, without limitation, any parenteral route, including intravenous, intra-arterial, intramuscular, subcutaneous, intradermal, intraperitoneal, intrathecal, as well as topically, orally, and by mucosal routes of delivery such as intranasal, inhalation, rectal, vaginal, buccal, and sublingual. In some embodiments, the pharmaceutical compositions of the invention are prepared for administration to vertebrate (e.g., mammalian) subjects in the form of liquids, including sterile, non-pyrogenic liquids for injection, emulsions, powders, aerosols, tablets, capsules, enteric coated tablets, or suppositories.

In some embodiments, compositions and methods are provided for the establishment (e.g., reestablishment (e.g., following medical treatment (e.g., chemotherapy, antibiotics, etc.), during or following a medical treatment, etc.)), of healthy gut microbiota in a subject. In some embodiments, compositions and methods are provided for the treatment of a condition or disease (e.g., autoimmune diseases (e.g., rheumatoid arthritis, celiac disease), allergic and atopic diseases (e.g., food allergies of all types, eosinophilic esophagitis, allergic rhinitis, allergic asthma, pet allergies, drug allergies), inflammatory conditions (e.g., inflammatory bowel disease, ulcerative colitis, Crohn’s disease), etc.) via the establishment of healthy gut microbiota. In some embodiments, administration of the compositions herein promotes growth of commensal gut bacteria (e.g., bacteria species of the family Lachnospiraceae (e.g., bacteria are of Clostridium Cluster XIVa, IV, and/or XVIII). In some embodiments, administration of the compositions herein inhibits growth of pathogenic bacteria. In some embodiments, methods herein comprise a step of assessing the levels of gut bacteria (e.g., commensal bacteria, pathogenic bacteria, etc.) in a subject (e.g., in the stool of a subject). In some embodiments, levels of gut bacterial are assessed before treatment with the compositions herein and/or after treatment with the compositions herein.

In some embodiments, compositions and methods are provided for the establishment (e.g., reestablishment (e.g., following medical treatment (e.g., chemotherapy, antibiotics, etc.), during or following a medical treatment, etc.)), of healthy levels of gut metabolites in a subject. In some embodiments, compositions and methods are provided for the treatment of a condition or disease (e.g., autoimmune diseases (e.g., rheumatoid arthritis, celiac disease), allergic and atopic diseases (e.g., food allergies of all types, eosinophilic esophagitis, allergic rhinitis, allergic asthma, pet allergies, drug allergies), inflammatory conditions (e.g., inflammatory bowel disease, ulcerative colitis, Crohn’s disease), etc.) via the establishment of healthy levels of gut metabolites. In some embodiments, administration of the compositions herein provides beneficial metabolites (e.g., SCFAs (e.g., butyrate)) and promotes the anabolism of beneficial metabolites within a subject. In some embodiments, methods herein comprise a step of assessing the levels of metabolites (e.g., SCFAs (e.g., butyrate)) in a subject (e.g., in the stool of a subject). In some embodiments, levels of gut metabolites are assessed before treatment with the compositions herein and/or after treatment with the compositions herein.

### Experimental

### Example 1 pMAA-b-pBMA

To deliver SFCAs (e.g., butyrate) into the gastrointestinal (GI) tract, block copolymers that can form water-suspendible micelles carrying a high content of butyrate in their core. pHPMA-b-pBMA was previously described (U.S. Pub. No. 2020/0048390; incorporated by reference in its entirety). Provided herein is an exemplary copolymer, pMAA-b-pBMA, which has an anionic block made of hydrophilic methacrylic acid (MAA), and spontaneously forms negatively-charged micelles (Neg-ButM) in an alkaline aqueous solution. The negative surface charge affects distribution and absorption when administered intragastrically. In particular, Neg-ButM showed slower release kinetics in the simulated gastric fluid, longer retention time in the GI tract, and more butyrate release in the mouse cecum, as compared to the neutral charge NtL-ButM.

Experiments were conducted during development of embodiments herein to investigate lymph node targeting of the negatively-charged Neg-ButM when injected subcutaneously (SC). The Neg-ButM showed superior accumulation and long-term retention in the draining LNs after SC administration, leading to substantial regulatory T cell (Tregs) induction. This affect may be useful in a number of inflammatory and immunological medical conditions.

### Synthesis of the pMAA-b-BMA

### Synthesis of N-(2-Hydroxyethyl) Methacrylamide (2)

To synthesize N-(2-hydroxyethyl) methacrylamide (HEMA, 2), ethanolamine (3.70 mL, 61.4 mmol, 2.0 eq), triethylamine (4.72 mL, 33.8 mmol, 1.1 eq) and 50 mL DCM were added into a 250 mL flask. After the system was cooled down by an ice bath, methacryloyl chloride (1, 3.00 mL, 30.7 mmol, 1.0 eq) was added dropwise under the protection of nitrogen. The reaction was allowed to warm up to room temperature and reacted overnight. Then the reaction mixture was concentrated by rotary evaporation and purified on a silica column using DCM/MeOH (MeOH fraction v/v from 0% to 5%). The product was obtained as colorless oil (3.42 g, 86.3%). MS (ESI). C6H11NO2, m/z calculated for [M+H]+: 129.08, found: 129.0. 1H-NMR (500 MHz, CDCl3) δ 1.93 (s, 3H), 3.43 (m, 2H), 3.71 (m, 2H), 5.32 (s, 1H), 5.70 (s, 1H), 6.44 (br s, 1H)

### Synthesis of N-(2-Butanoyloxyethyl) Methacrylamide (3)

To synthesize N-(2-butanoyloxyethyl) methacrylamide (BMA, 3), N-(2-hydroxyethyl) methacrylamide (3.30 mL, 25.6 mmol, 1.0 eq), triethylamine (7.15 mL, 51.2 mmol, 2.0 eq) and 50 mL DCM were added into a 250 mL flask. After the reaction system was cooled down by an ice bath, butyric anhydride (5.00 mL, 30.7 mmol, 1.2 eq) was added dropwise under the protection of nitrogen. The system was allowed to react overnight. The reaction mixture was filtered and washed by NH4Cl solution, NaHCO3 solution, and water. After dried by anhydrous MgSO4, the organic layer was concentrated by rotary evaporation and purified on a silica column using DCM/MeOH (MeOH fraction v/v from 0% to 5%). The product was obtained as pale yellow oil (4.56 g, 89.6%). MS (ESI). C10H17NO3, m/z calculated for [M+H]+: 199.12, found: 199.1. 1H-NMR (500 MHz, CDCl3) δ 0.95 (t, 3H), 1.66 (m, 2H), 1.97 (s, 3H), 2.32 (t, 2H), 3.59 (dt, 2H), 4.23 (t, 2H), 5.35 (s, 1H), 5.71 (s, 1H), 6.19 (br s, 1H)

### Synthesis of pMAA (7) and pMAA-b-pBMA (8)

pMAA was prepared using 2-cyano-2-propyl benzodithioate as the RAFT chain transfer agent and 2,2′-Azobis(2-methylpropionitrile) (AIBN) as the initiator. Briefly, methacrylic acid (MAA) (4.0 mL, 47.2 mmol, 1.0 eq), 2-cyano-2-propyl benzodithioate (104.4 mg, 0.472 mmol, 1/100 eq), and AIBN (19.4 mg, 0.118 mmol, 1/400 eq) were dissolved in 20 mL MeOH in a 50 mL Schlenk tube. The reaction mixture was subjected to four freeze-pump-thaw cycles. The polymerization was conducted at 70° C. for 24 h. The polymer was precipitated in hexanes and dried in the vacuum oven overnight. The product was obtained as light pink solid (4.0 g, 100 %). 1H-NMR (500 MHz, DMSO-d6) δ 0.8-1.2 (m, 3H, backbone CH3), 1.5-1.8 (m, 2H, backbone CH2), 7.4-7.8 (three peaks, 5H, aromatic H), 12.3 (m, 1H, CO-OH)

The block copolymer pMAA-b-pBMA (8) was prepared using (7) pMAA as the macro-RAFT chain transfer agent and (3) N-(2-butanoyloxyethyl) methacrylamide (BMA) as the monomer of the second RAFT polymerization. Briefly, pMAA (0.50 g, 0.058 mmol, 1.0 eq), N-(2-butanoyloxyethyl) methacrylamide (1.47 g, 7.38 mmol, 127 eq), and AIBN (2.4 mg, 0.015 mmol, 0.25 eq) were dissolved in 10 mL MeOH in a 25 mL Schlenk tube. The reaction mixture was subjected to four freeze-pump-thaw cycles. The polymerization was conducted at 70° C. for 24 h. The polymer was precipitated in hexanes and dried in the vacuum oven overnight. The product was obtained as light pink solid (1.5 g, 70%). 1H-NMR (500 MHz, DMSO-d6) δ 0.8-1.1 (m, 6H, CH2—CH3 (BMA), and backbone CH3), 1.5-1.7 (m, 4H, CH2—CH2 (BMA) and backbone CH2), 2.3 (m, 2H, CO—CH2 (BMA)), 3.2 (m, 2H, NH—CH2 (BMA)), 4.0 (m, 2H, O—CH2 (BMA)), 7.4 (m, 1H, NH), 12.3 (m, 1H, CO—OH)

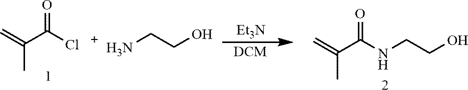

Scheme 1 Synthesis of N-(2-hydroxyethyl) methacrylamide (HEMA, 2)

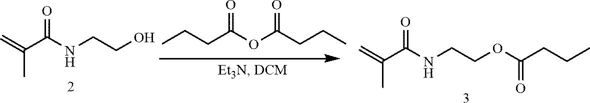

Scheme 2 Synthesis of N-(2-butanoyloxyethyl) methacrylamide (BMA, 3)

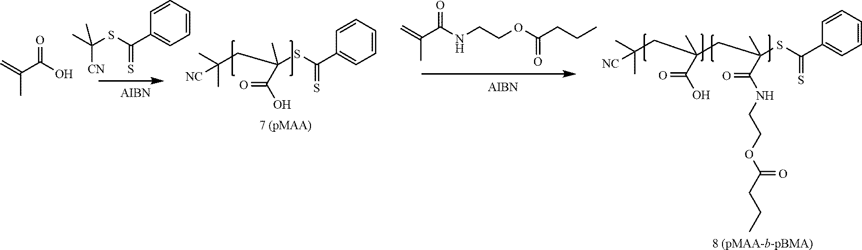

Scheme 3 Synthetic route of pMAA-b-pBMA (8)

### Fabrication of Neg-ButM

Neg-ButM micelle was prepared by base titration (Refs. A8,A9; incorporated by reference in their entireties). 60 mg of pMAA-b-pBMA polymer was added to 8 mL of 1 × PBS under vigorous stirring. Sodium hydroxide solution in equivalent to methacrylic acid was added to the polymer solution in three portions during 2 h. After adding base solution, the polymer solution was allowed stirring at room temperature overnight. After that time, 1 × PBS was added to reach the target volume and the solution was filtered through 0.22 µm filter and the pH of the solution was checked to make sure it was neutral. The size of the micelle was measured by dynamic light scattering (DLS).

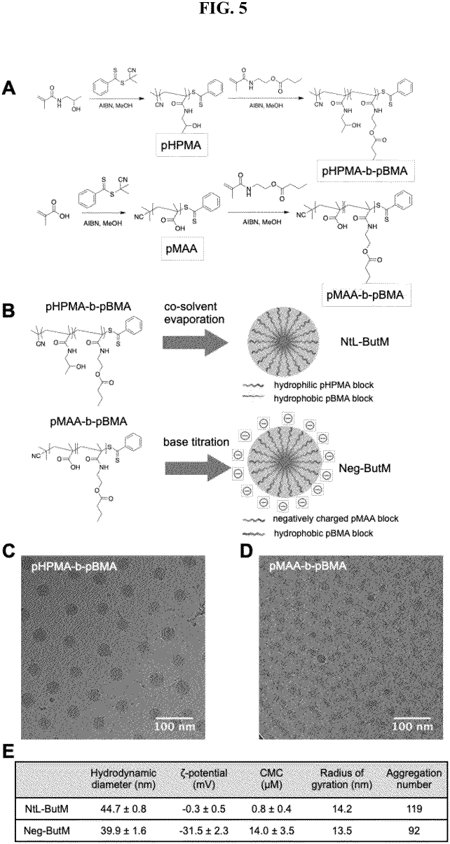

pMAA-b-pBMA cannot be formulated into micelles by this method because of the formation of intramolecular hydrogen bonds between pMAA chains (Ref. A10; incorporated by reference in its entirety). Such bonding can, however, be disrupted when a strong base, here NaOH, is titrated into the mixture of pMAA-b-pBMA polymer to change methacrylic acid into ionized methacrylate (Refs. A8, A9, A11; incorporated by reference in their entireties). Upon base titration, pMAA-b-pBMA polymer can then self-assembled into negatively charged micelles (Neg-ButM) that have size of 39.9 ± 1.6 nm, measured by dynamic light scattering (DLS). Their low polydispersity index below 0.1 indicated the monodispersity of those micelles (FIG. 5). The Neg-ButM has a ζ-potential of -31.5 ± 2.3 mV due to the ionization of methacrylic acid. It was reported that negative charged nanoparticles could stay in the GI tract for a longer time due to the stronger adherent effect to the gut mucosa (Refs. A10, A12-A13; incorporated by reference in their entireties). In addition, cryogenic electron microscopy (CryoEM) revealed the detailed structure of micelles, especially the core structure as made of pBMA, which were more condensed with higher contrast. CryoEM images indicated the diameter of the core of NtL-ButM was 30 nm, while Neg-ButM had a smaller core diameter of 15 nm (FIGS. 5C, D). To obtain the critical micelle concentrations (CMC) of NtL-ButM and Neg-ButM, which indicates the likelihood of formation and dissociation of micelles in aqueous solutions, pyrene was added during the formulation and the fluorescence intensity ratio between the first and third vibronic bands of pyrene was plotted to calculate the CMC (Refs. A14-A15; incorporated by reference in their entireties). Results showed that Neg-ButM had a higher CMC of 14.0 ± 3.5 µM, compared to the CMC of NtL-ButM, which was was 0.8 ± 0.4 µM (FIG. 5E). The higher CMC indicated that Neg-ButM micelles would be easier to dissociate in solution, possibly because the surface charge made the micellar structure unstable compared to neutral micelle NtL-ButM. In addition, the inventors did small angel X-ray scattering (SAXS) analysis on both micelles in order to obtain the aggregation number, which were 119 for NtL-ButM, and 92 for Neg-ButM (FIG. 5E).

### In Vitro Release Kinetics

Simulated gastric fluid and simulated intestinal fluid were as described before (Refs. A16-A17; incorporated by reference in their entireties). For ex vivo hydrolysis study, NtL-ButM or Neg-ButM was added to simulated gastric fluid, or simulated intestinal fluid at a final concentration of 2 mg/mL at 37° C. At pre-determined time points, 20 µL of the solution was transferred into 500 µL of water:acetonitrile 1:1 v/v. The sample was centrifuged using Amicon Ultra (Merck, 3 kDa molecular mass cutoff) at 13,000 × g for 15 min, to remove polymers. The filtrate was stored at -80° C. before derivatization.

Samples were prepared and derivatized as describe in the literature (Refs. A18-A19; incorporated by reference in their entireties). 3-nitrophenylhydrazine (NPH) stock solution was prepared at 0.02 M in water:acetonitrile 1:1 v/v. 1-Ethyl-3-(3-dimethylaminopropyl)carbodiimide (EDC) stock solution was prepared at 0.25 M in water:acetonitrile 1:1 v/v. 4-methylvaleric acid was added as internal standard. Samples were mixed with NPH stock and EDC stock at 1:1:1 ratio by volume. The mixture was heated by heating block at 60° C. for 30 min. Samples were transferred into HPLC vials and stored at 4° C. before analysis.

LC conditions: The instrument used for quantification of butyrate was Agilent 1290 UHPLC. Column: Thermo Scientific C18 4.6 × 50 mm, 1.8 m particle size, at room temperature. Mobile phase A: water with 0.1% v/v formic acid. Mobile phase B: acetonitrile with 0.1% v/v formic acid. Injection volume: 5.0 µL. Flow rate: 0.5 mL/min. Gradient of solvent: 15% mobile phase B at 0.0 min; 100% mobile phase B at 3.5 min; 100% mobile phase B at 6.0 min; 15% mobile phase B at 6.5 min.

Liquid chromatography with tandem mass spectrometry (LC-MS/MS) method: The instrument used to detect butyrate was Agilent 6460 Triple Quad MS-MS. Both derivatized butyrate-NPH and 4-methylvaleric-NPH were detected in negative mode. The MS conditions were optimized on pure butyrate-NPH or 4-methylvaleric-NPH at 1 mM. The fragment voltage was 135 V and collision energy was set to 18 V. Multiple reaction monitoring (MRM) of 222 → 137 was assigned to butyrate, and MRM of 250 → 137 was assigned to 4-methylvaleric acid as internal standard. The ratio between MRM of butyrate and 4-methylvaleric acid was used to quantify the concentration of butyrate.

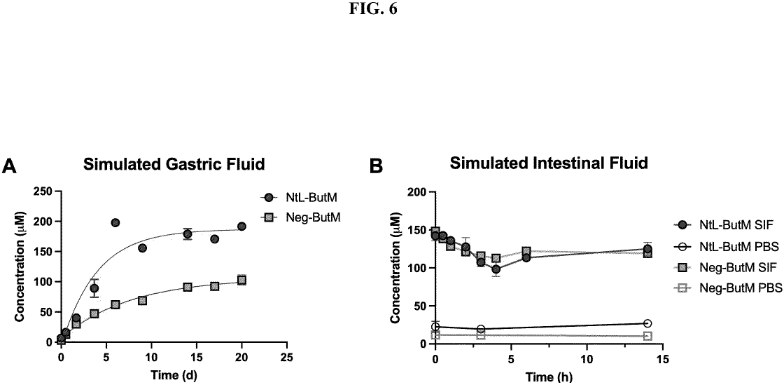

In the simulated gastric fluid, both Neg-ButM and NtL-ButM showed negligible release of butyrate within hours, and sustained slow release over 3 weeks, while Neg-ButM had even slower release rate than NtL-ButM (FIG. 6A). The anionic surface of Neg-ButM in the acidic environment is likely responsible for the resistance to the hydrolysis of the BMA core. By contrast, in simulated intestinal fluid, both micelles released the most of their butyrate within minutes in the presence of a high concentration of the esterase pancreatin (FIG. 6B).

### In Vivo Biodistribution and Pharmacokinetics

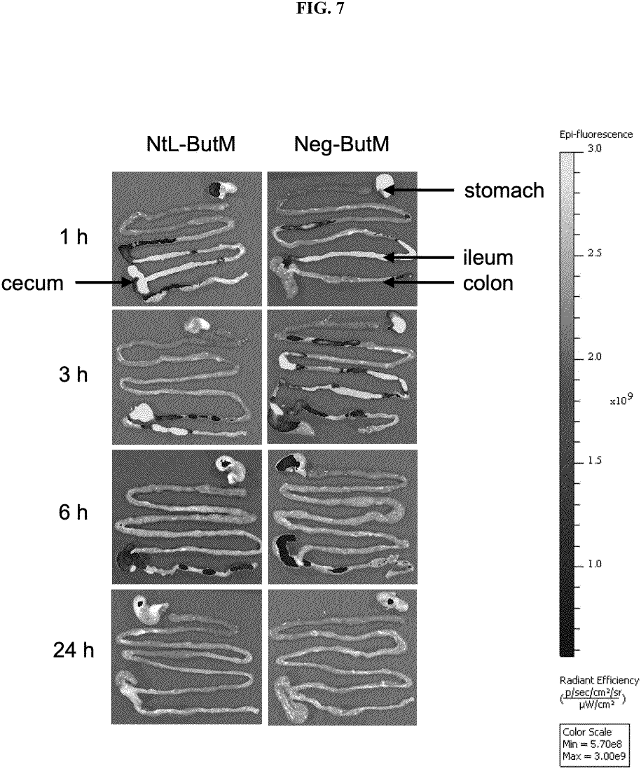

To investigate how the butyrate would be delivered from these micelles when administered orally, the inventors first studied the biodistribution of fluorescently-labeled NtL-ButM and Neg-ButM on mice via In Vivo Imaging System (IVIS) (FIG. 7). The IVIS results validated that the polymeric micelles were retained in the mouse GI tract for more than 6 hr after gavage. The neutral micelle NtL-ButM passed through the stomach and small intestine within 2 hr and accumulated in the cecum. However, negatively charged Neg-ButM accumulated in the stomach first and then gradually traveled through the small intestine to the cecum. Overall, Neg-ButM had a longer retention time in the stomach and small intestine. Both micelles were cleared from the GI tract within 24 hr after administration. In addition, we measured the fluorescence signal from other major organs and plasma by IVIS, as well as the butyrate concentration in the plasma by LC-MS/MS. The signals were all below the detection limit from both methods, suggesting that there was negligible absorption of these butyrate micelles into the blood circulation from the intestine, consistent with our desire to deliver butyrate to the gut and to avoid any complexities of systemic absorption of the polymer or micelles.

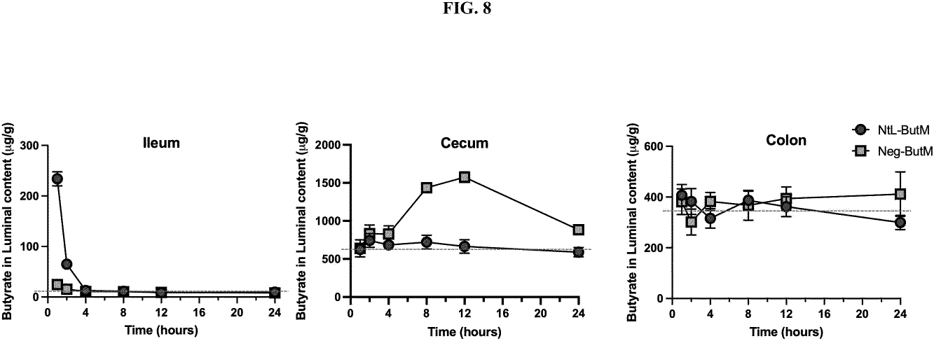

The inventors also measured the butyrate levels in the mouse GI tract affected by polymeric micelles. Both LC-UV and LC-MS/MS methods were used to measure the butyrate concentrations in the fecal contents of ileum, cecum, or colon of mice orally administered with either NtL-ButM or Neg-ButM (Refs. A18-A19; incorporated by reference in their entireties), while LC-MS/MS method was mainly used to measure the butyrate concentration in ileum because the baseline concentration in ileum was too low for UV detector. The results showed NtL-ButM dramatically increased the butyrate concentration in the ileum for up to 2 hr after gavage, this was short lived and butyrate concentration did not increase in either the cecum or colon (FIG. 8). Interestingly, Neg-ButM raised butyrate concentrations by 3-fold in the cecum starting from 4 hr after gavage and lasting for at least another 8 hr but not in the ileum or colon. Due to the different butyrate release behavior in vivo from the two butyrate micelles, the combined dosage of NtL-ButM and Neg-ButM could cover the most section of GI tract and last for longer time when applying on the animal disease model.

### Neg-ButM Accumulated in the Draining LNs After Subcutaneous (SC) Injections

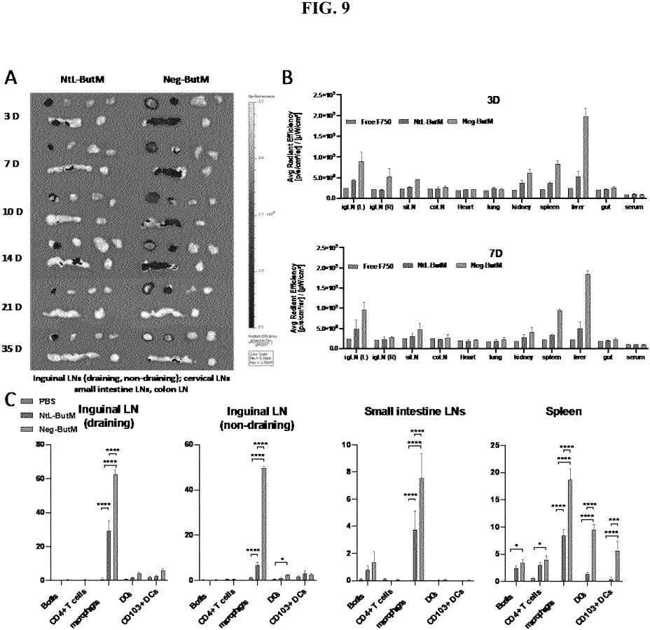

The lymphatic vessels exhibit wider inter-endothelial junctions than vascular capillaries, allowing larger carriers (10-100 nm) to enter more efficiently from interstitium (Ref. A20; incorporated by reference in its entirety). In addition, the neutral or positively charged vehicles are more likely to get trapped in the extracellular matrix of negatively-charged interstitium (Ref. A21; incorporated by reference in its entirety). Herein, the investors demonstrated using the Neg-ButM as the novel platform to target butyrate to the LNs through SC administration. To track the micelles’ biodistribution in vivo, the investors fluorescently labeled polymers and administered either NtL-ButM, Neg-ButM, or equivalent free dyes subcutaneously into the abdomen of specific-pathogen-free (SPF) C3H/HeJ mice. At various time points following injection, they collected blood and tissues, and quantified the fluorescent signals using an In Vivo Imaging System (IVIS). The investors also digested LNs and spleen into single cell suspensions and analyzed cellular biodistribution using flow cytometry. It was observed that the Neg-ButM accumulated and was retained in the draining inguinal LNs for a remarkably long time (over 35 days), which was not observed for the NtL-ButM (FIGS. 9A, B). At the cellular level, both micelles were mostly taken up by the macrophages in the LNs, while Neg-ButM was taken up by more cells compared to NtL-ButM (FIG. 9C).

### Neg-ButM Inhibited LPS-Induced Activation of APCs in the dLNs.

In order to assess the ability of lymph node targeted Neg-ButM in suppressing the activation of APCs, the investors tested on a mouse model of subcutaneous lipopolysaccharide (LPS) stimulation and evaluated on the activation marker including CD40 and CD86 on the major APCs in the draining LNs. Mice were injected with either PBS, sodium butyrate (NaBut), NtL-ButM, or Neg-ButM subcutaneously at the abdomen site. On Day 6, Mice were challenged with LPS on the same injection site, and sacrificed the next day for the cellular analysis.

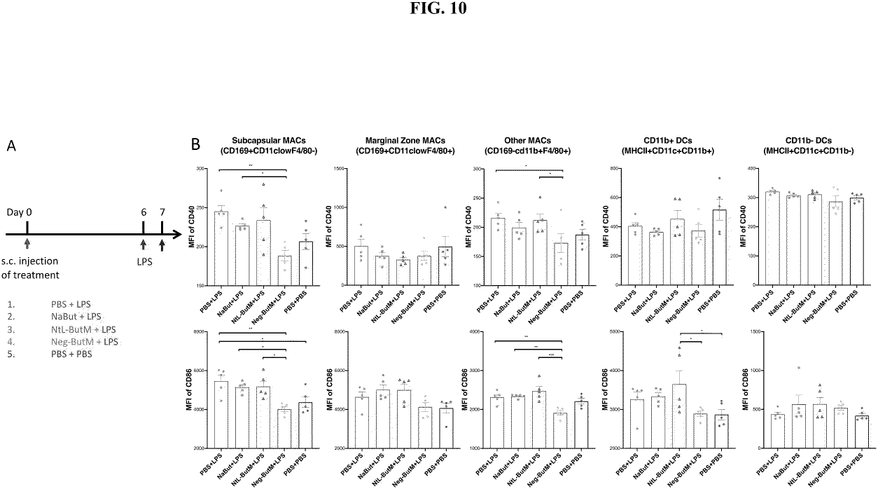

The Neg-ButM treatment significantly inhibited the over expression of CD40 and CD86 on the subcapsular macrophage and the CD169-CD1 1b+F4/80+ macrophages in the draining LNs upon LPS stimulation (FIG. 10). These macrophages were also the major uptaker of the Neg-ButM from the previous cellular biodistribution study. The Neg-ButM also reduced CD86 expression on the CD11b+ dendritic cells. In contract, neither the NtL-ButM, nor sodium butyrate showed any significant suppression on the APC activations.

### Neg-ButM Induced Suppressive Tregs in the Draining LNs.

It has been shown that microbiome-derived short-chain fatty acids (SCFAs) facilitate extrathymic differentiation of Tregs (Refs. A5-A6; incorporated by reference in their entireties). In particular, butyrate has long been considered a promising therapeutic candidate due to its function of inducing gut-localized Tregs (Refs. A5-A6; incorporated by reference in their entireties).

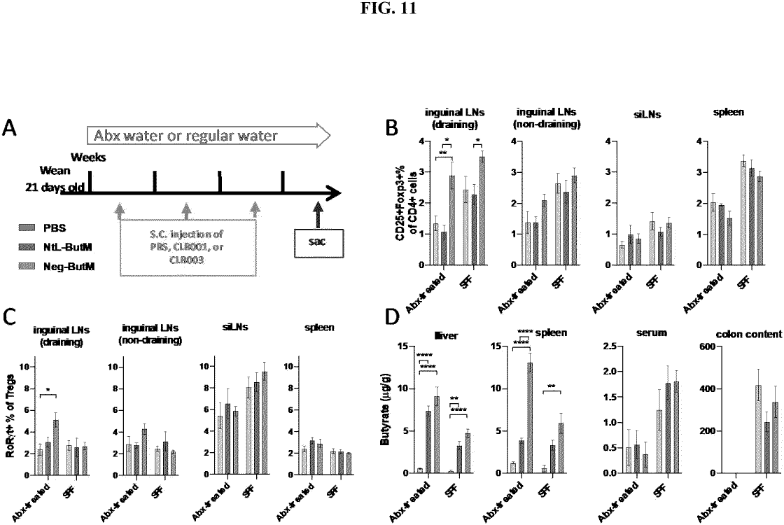

Peripherally-derived Tregs are induced most efficiently in the lymph nodes (LNs), yet current therapies do not efficiently target the LN. Here, the investors demonstrated the Treg induction in the draining LNs through SC administration of Neg-ButM. To investigate the potential of Treg induction in vivo, the investors administered PBS, NtL-ButM, or Neg-ButM to SPF C57BL/6 Foxp3^{GFP+} mice by s.c. injection weekly for 3 weeks. A week after the final dose, they sacrificed mice and isolated cells from relevant LNs and spleen to analyze Treg populations by flow cytometry (FIG. 11A). The investors also measured the butyrate concentrations in liver, spleen, serum, and colon content through LC-MS/MS. The investors demonstrated that antibiotic-treated mice have a lower frequency of Tregs in both LNs and the spleen than SPF mice. However, the Neg-ButM treatment significantly increased and restored Treg population in the draining inguinal LNs compared to PBS or NtL-ButM treated mice. In the SPF mice, Neg-ButM further increased Treg populations in the draining LNs (FIG. 11B). a substantial increase of RORγt+ Tregs by Neg-ButM in the draining LNs was also observed (FIG. 11C), which is a subset of Tregs induced by microbes or SCFAs in the gut and suppresses the Th2 response (Ref. A22; incorporated by reference in its entirety). The micelles also release butyrate in the liver and spleen (FIG. 11D), suggesting their potential entrance into systemic circulation.

### Example 2 Combined pHPMA-b-pBMA and pMAA-b-pBMA micelles

### I. Materials and Methods

### Materials for Polymer Synthesis

N-hydroxyethyl) methacrylamide (HPMA) monomer was obtained from Sigma-Aldrich or Polysciences, Inc. Solvents including dichloromethane, methanol, hexanes, and ethanol were ACS reagent grade and were obtained from Fisher Scientific. All other chemicals were obtained from Sigma-Aldrich.

### Synthesis of N-(2-Hydroxyethyl) Methacrylamide (2)

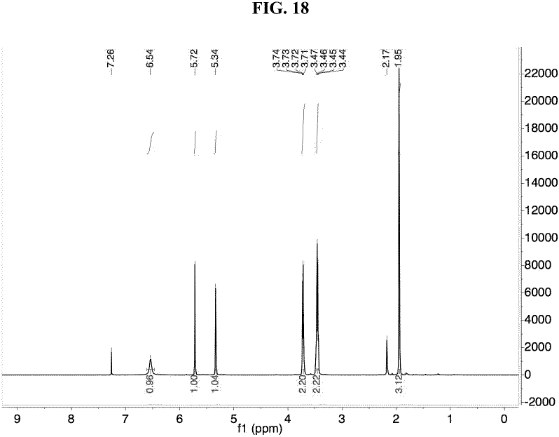

To synthesize N-(2-hydroxyethyl) methacrylamide (HEMA, 2), ethanolamine (3.70 mL, 61.4 mmol, 2.0 eq), triethylamine (4.72 mL, 33.8 mmol, 1.1 eq) and 50 mL DCM were added into a 250 mL flask. After the system was cooled by an ice bath, and methacryloyl chloride (1, 3.00 mL, 30.7 mmol, 1.0 efq) was added dropwise under the protection of nitrogen. The reaction was allowed towarm to room temperature and reacted overnight. Then the reaction mixture was concentrated by rotary evaporation and purified on a silica column using DCM/MeOH (MeOH fraction v/v from0% to 5%). The product was obtained as a colorless oil (3.42 g, 86.3%). MS (ESI). C6H11NO2, m/zcalculated for [M+H]⁺: 129.08, found: 129.0. ¹H-NMR (500 MHz, CDCl3) δ 1.93 (s, 3H), 3.43 (m, 2H), 3.71 (m, 2H), 5.32 (s, 1H), 5.70 (s, 1H), 6.44 (br s, 1H) (FIG. 18).

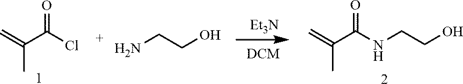

Scheme 1. Synthesis of N-(2-hydroxyethyl) methacrylamide (HEMA, 2)

### Synthesis of N-(2-Butanoyloxyethyl) Methacrylamide (3)

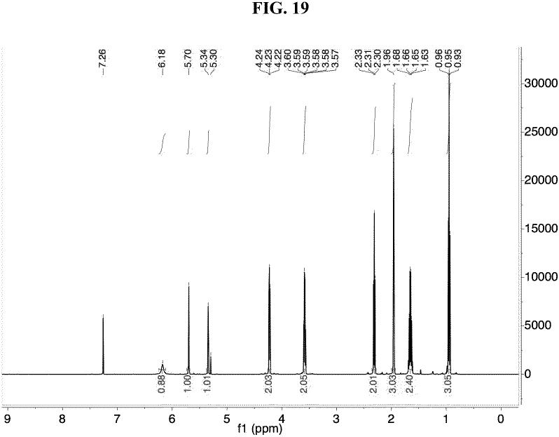

To synthesize N-(2-butanoyloxyethyl) methacrylamide (BMA, 3), N-(2-hydroxyethyl) methacrylamide (3.30 mL, 25.6 mmol, 1.0 eq), triethylamine (7.15 mL, 51.2 mmol, 2.0 eq) and 50 mL DCM were added into a 250 mL flask. After the reaction system was cooled by an ice bath, butyric anhydride (5.00 mL, 30.7 mmol, 1.2 eq) was added dropwise under the protection of nitrogen. The system was allowed to react overnight. The reaction mixture was filtered and washed by NH₄Cl solution, NaHCO3 solution, and water. After drying by anhydrous MgSO4, the organic layer was concentrated by rotary evaporation and purified on a silica column using DCM/MeOH (MeOH fraction v/v from 0% to 5%). The product was obtained as a pale-yellow oil (4.56 g, 89.6%). MS (ESI). C10H17NO3, m/z calculated for [M+H]⁺: 199.12, found: 199.1. ¹H-NMR(500 MHz, CDCl3) δ 0.95 (t, 3H), 1.66 (m, 2H), 1.97 (s, 3H), 2.32 (t, 2H), 3.59 (dt, 2H), 4.23 (t, 2H), 5.35 (s, 1H), 5.71 (s, 1H), 6.19 (br s, 1H) (FIG. 19).

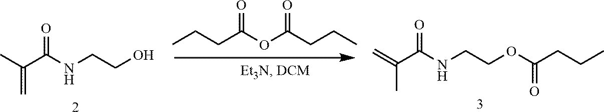

Scheme 2. Synthesis of N-(2-butanoyloxyethyl) methacrylamide (BMA, 3)

### Synthesis of Poly(2-Hydroxypropyl Methacrylamide) (pHPMA, 5)

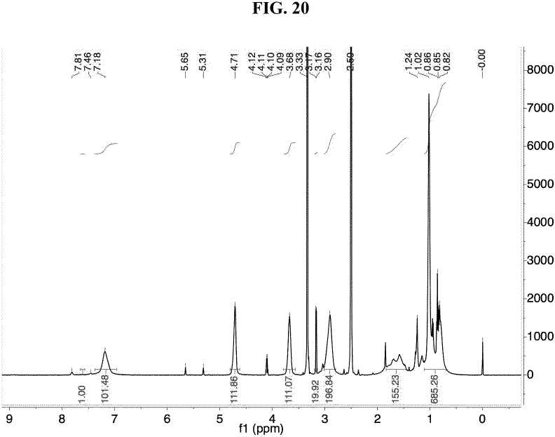

pHPMA was prepared using 2-cyano-2-propyl benzodithioate as the RAFT chain transfer agent and 2,2′-Azobis(2-methylpropionitrile) (AIBN) as the initiator. Briefly, HPMA (4, 3.0 g, 20.9 mmol, 1.0 eq), 2-cyano-2-propyl benzodithioate (28.3 mg, 0.128 mmol, 1/164 eq), and AIBN (5.25 mg, 0.032 mmol, 1/656 eq) were dissolved in 10 mL MeOH in a 25 mL Schlenk tube. The reaction mixture was subjected to four freeze-pump-thaw cycles. The polymerization was conducted at 70° C. for 30 hr. The polymer was precipitated in a large volume of petroleum ether and dried in the vacuum chamber overnight. The product obtained was a light pink solid (1.8 g, 60 %). ¹H- NMR (500 MHz, DMSO-d6) δ 0.8-1.2 (m, 6H, CH(OH)—CH3 and backbone CH3), 1.5-1.8 (m, 2H, backbone CH2), 2.91 (m, 2H, NH-CH2), 3.68 (m, 1H, C(OH)—H), 4.70 (m, 1H, CH-OH), 7.18 (m, 1H, NH) (FIG. 20).

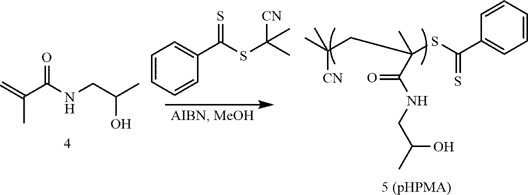

Scheme 3. Synthesis of poly(2-hydroxypropyl methacrylamide) (pHPMA, 5)

### Synthesis of pHPMA-b-pBMA (6)

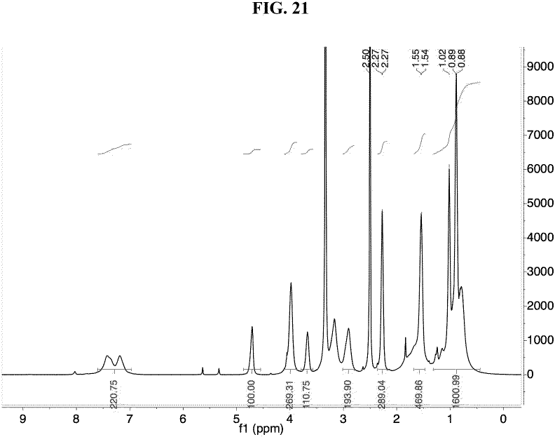

The block copolymer pHPMA-b-pBMA was prepared using pHPMA (5) as the macro-RAFT chaintransfer agent and N-(2-butanoyloxyethyl) methacrylamide (3) as the monomer of the second RAFT polymerization. Briefly, pHPMA (1.50 g, 0.105 mmol, 1.0 eq), N-(2-butanoyloxyethyl) methacrylamide (4.18 g, 21.0 mmol, 200 eq), and AIBN (8.3 mg, 0.050 mmol, 0.50 eq) were dissolved in 10 mL MeOH in a 50 mL Schlenk tube. The reaction mixture was subjected to four freeze-pump-thaw cycles. The polymerization was conducted at 70° C. for 20 hr. The polymer wasprecipitated in petroleum ether and dried in the vacuum chamber overnight. The product obtained was a light pink solid (4.22 g, 74%). ¹H-NMR (500 MHz, DMSO-d6) δ 0.80-1.1 (m, 9H, CH(OH)— CH3 (HPMA), CH2—CH3 (BMA), and backbone CH3), 1.55 (m, 4H, CH2—CH2 (BMA) and backbone CH2), 2.28 (m, 2H, CO—CH2 (BMA)), 2.91 (m, 2H, NH—CH2 (HPMA)), 3.16 (m, 2H, NH—CH2 (BMA)), 3.67 (m, 1H, CH(OH)—H), 3.98 (m, 2H, O—CH2 (BMA)), 4.71 (m, 1H, CH—OH (HPMA)), 7.19 (m, 1H, NH), 7.44 (m, 1H, NH) (FIG. 21).

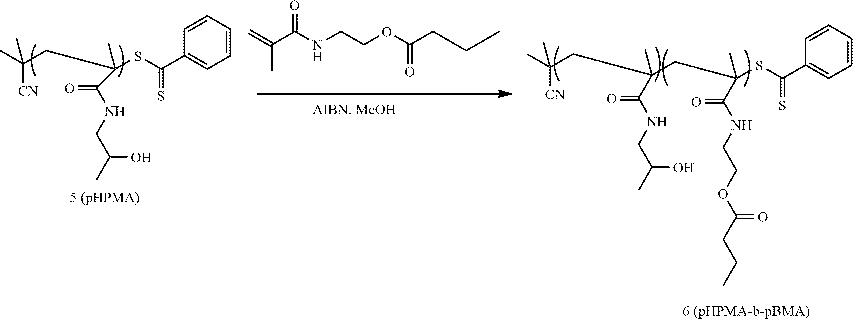

Scheme 4. Synthesis of pHPMA-b-pBMA (6)

### Synthesis of pMAA (7) and pMAA-b-pBMA (8)

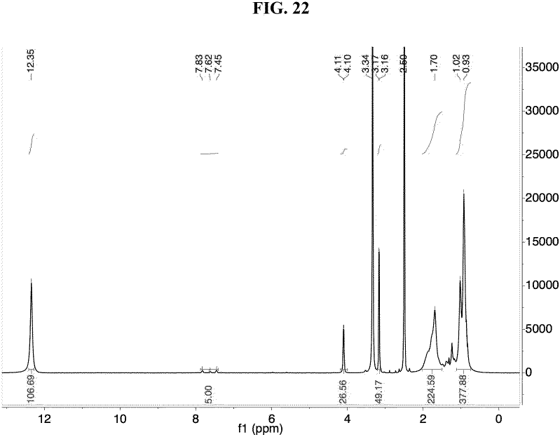

pMAA was prepared using 2-cyano-2-propyl benzodithioate as the RAFT chain transfer agentand AIBN as the initiator. Briefly, methacrylic acid (MAA) (4.0 mL, 47.2 mmol, 1.0 eq), 2-cyano- 2-propyl benzodithioate (104.4 mg, 0.472 mmol, 1/100 eq), and AIBN (19.4 mg, 0.118 mmol, 1/400 eq) were dissolved in 20 mL MeOH in a 50 mL Schlenk tube. The reaction mixture was subjected to four freeze-pump-thaw cycles. The polymerization was conducted at 70° C. for 24 hr.The polymer was precipitated in hexanes and dried in the vacuum oven overnight. The product obtained was a light pink solid (4.0 g, 100 %). ¹H-NMR (500 MHz, DMSO-d6) δ 0.8-1.2 (m, 3H, backbone CH₃), 1.5-1.8 (m, 2H, backbone CH2), 7.4-7.8 (three peaks, 5H, aromatic H), 12.3 (m, 1H, CO—OH) (FIG. 22).

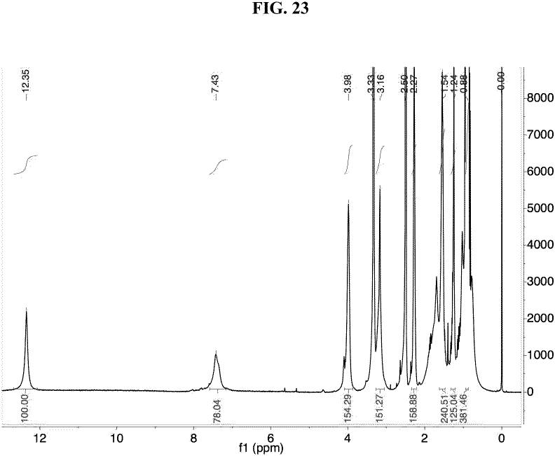

The block copolymer pMAA-b-pBMA (8) was prepared using (7) pMAA as the macro-RAFT chaintransfer agent and (3) N-(2-butanoyloxyethyl) methacrylamide (BMA) as the monomer of the second RAFT polymerization. Briefly, pMAA (0.50 g, 0.058 mmol, 1.0 eq), N-(2-butanoyloxyethyl)methacrylamide (1.47 g, 7.38 mmol, 127 eq), and AIBN (2.4 mg, 0.015 mmol, 0.25 eq) were dissolved in 10 mL MeOH in a 25 mL Schlenk tube. The reaction mixture was subjected to four freeze-pump-thaw cycles. The polymerization was conducted at 70° C. for 24 hr. The polymer wasprecipitated in hexanes and dried in the vacuum oven overnight. The product obtained was a lightpink solid (1.5 g, 70%). ¹H-NMR (500 MHz, DMSO-d6) δ 0.8-1.1 (m, 6H, CH2—CH3 (BMA), and backbone CH3), 1.5-1.7 (m, 4H, CH2—CH2 (BMA) and backbone CH2), 2.3 (m, 2H, CO—CH2 (BMA)), 3.2 (m, 2H, NH—CH2 (BMA)), 4.0 (m, 2H, O—CH2 (BMA)), 7.4 (m, 1H, NH), 12.3 (m, 1H, CO—OH) (FIG. 23).

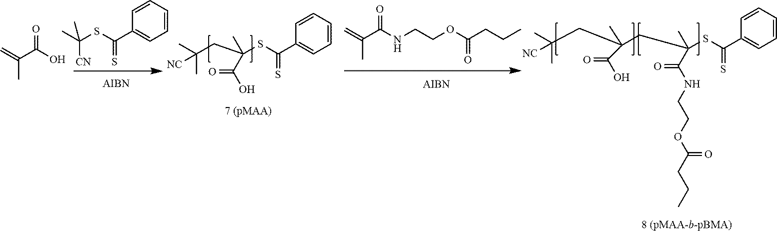

Scheme 5. Synthetic route of pMAA-b-pBMA (8)

### Synthesis of N₃-PEG₄-MA (9) and Azide-PEG Polymer

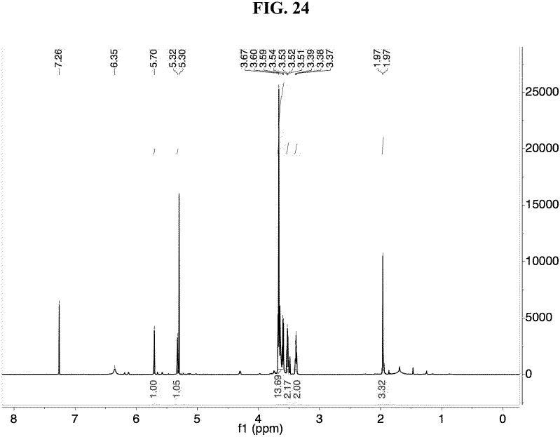

In order to include an azide group into pHPMA-b-pBMA or pMAA-b-pBMA polymers, monomer N-(2-(2-(2-(2-azidoethoxy)ethoxy)ethoxy)ethyl) methacrylamide (9) was synthesized and used inthe copolymerization with HPMA or MAA to obtain the hydrophilic block with azide function. N3—PEG4—NH2 (0.5 g, 2.14 mmol, 1.0 eq) and triethylamine (0.60 mL, 4.3 mmol, 2.0 eq) were dissolved in anhydrous DCM. After the reaction system was cooled by an ice bath, methacrylic chloride (0.42 mL, 2.6 mmol, 1.2 eq) was added dropwise under the protection of nitrogen. The system was allowed to react overnight. The reaction mixture was filtered and washed by NH4Cl solution, NaHCO3 solution, and water. After being dried by anhydrous MgSO4,the organic layer was concentrated by rotary evaporation and purified on a silica column using DCM/MeOH (MeOH fraction v/v from 0% to 5%). The product obtained was a pale-yellow oil (0.47 g, 73 %). MS (ESI). C₁₂H₂₂ N₄O₄, m/z calculated for [M+H]⁺: 287.16, found: 287.2. ¹H-NMR (500 MHz, CDCl₃) δ 6.35 (br, 1H), 5.70 (s, 1H), 5.32 (s, 1H), 3.55-3.67 (m, 12H), 3.52 (m, 2H), 3.38 (t, 2H), 1.97 (s, 3H) (FIG. 24). Monomer N3-PEG4-MA was mixed with HPMA or MAA in a 2:98 wt:wtratio during the RAFT polymerization to obtain N3-pHPMA or N3-pMAA. Then, the second block of BMA was added to the macro initiator to obtain N3-pHPMA-b-pBMA or N3-pMAA-b-pBMA, respectively. The synthesis procedures were the same as the previous description.

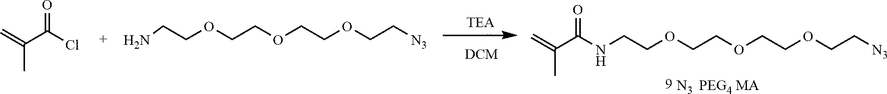

Scheme 6. Synthesis of hydrophilic azide monomer N3-PEG4-MA (9)

### Synthesis of N-Hexyl Methacrylamide (10) and Control Polymer

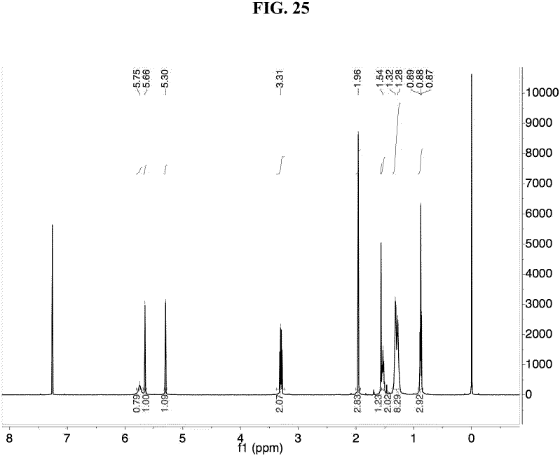

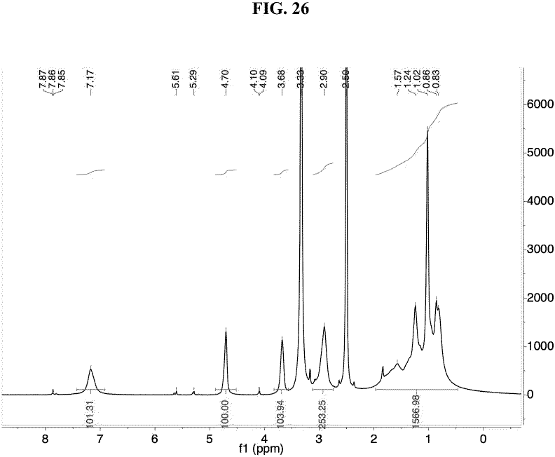

In order to synthesize a control polymer that did not contain butyrate ester, monomer N-hexyl methacrylamide (11) was synthesized and used in the polymerization of hydrophobic block. Hexanamine (5.8 mL, 46.0 mmol, 1.5 eq), triethylamine (4.7 mL, 33.8 mmol, 1.1 eq) and 50 mL DCM were added into a 250 mL flask. After the system was cooled by an ice bath, methacryloyl chloride (3.0 mL, 30.7 mmol, 1.0 eq) was added dropwise under the protection of nitrogen. The reaction was allowed to warm to room temperature and reacted overnight. Then thereaction mixture was concentrated by rotary evaporation and purified on a silica column using DCM/MeOH (MeOH fraction v/v from 0% to 5%). The product obtained was a colorless oil (4.6 g,88%). MS (ESI). C₁₁H₂₁NO, m/z calculated for [M+H]⁺: 184.16, found: 184.2. ¹H-NMR (500 MHz,CDCl₃) δ 5.75 (br, 1H), 5.66 (s, 1H), 5.30 (s, 1H), 3.31 (t, 2H), 1.96 (s, 3H), 1.54 (m, 2H), 1.28-1.32 (m, 8H), 0.88 (t, 3H) (FIG. 25). After the synthesis of pHPMA or pMAA, monomer N-hexyl methacrylamide (11) was used in the polymerization of second block instead of N-(2-butanoyloxyethyl) methacrylamide to obtain control polymers as pHPMA-b-pHMA or pMAA-b- pHMA, respectively (FIG. 26). The synthesis procedures were the same as described above.

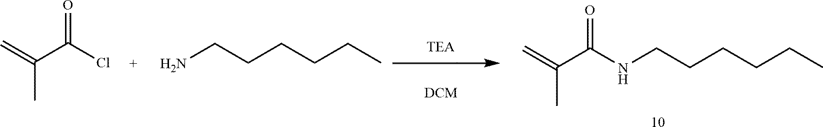

Scheme 7. Synthesis of hydrophobic control monomer N-hexyl methacrylamide (10)

### Formulation of Polymeric Micelles

NtL-ButM micelle was formulated by cosolvent evaporation method. 80 mg of pHPMA-b-pBMA polymer was dissolved in 10 mL of ethanol under stirring. After the polymer was completely dissolved, the same volume of 1 × PBS was added slowly to the solution. The solution was allowed to evaporate at room temperature for at least 6 hr until ethanol was removed. After the evaporation, the NtL-ButM solution was filtered through a 0.22 µm filter and stored at 4° C. The size of the micelles was measured by DLS.

Neg-ButM micelle was prepared by base titration (Refs. B27, B28; incorporated by reference in their entireties). 60 mg of pMAA-b-pBMA polymer was added to 8 mL of 1 × PBS under vigorous stirring. Sodium hydroxide solution in molar equivalentto methacrylic acid was added to the polymer solution in three portions over the course of 2 hr. After adding base solution, the polymer solution was stirred at room temperature overnight. 1 × PBS was then added to reach the target volume and the solution was filtered through a 0.22 µm filter. The pH of the solution was checked to confirm it was neutral, and the size of the micelles was measured by DLS.

### Dynamic Light Scattering (DLS) Characterizations of Micelles

DLS data was obtained from a Zetasizer Nano ZS90 (Malvern Instruments). Samples were diluted400 times in 1 × PBS and 700 µL was transferred to a DLS cuvette for data acquisition. The intensity distributions of DLS were used to determine the hydrodynamic diameter of micelles. Forzeta-potential data, micelles were diluted 100 times in 0.1 × PBS (1:10 of 1 × PBS to MilliQ water)and transferred to disposable folded capillary zeta cells for data acquisition.

### Cryogenic Electron Microscope Imaging of Micelles

CryoEM images were acquired on a FEI Talos 200 kV FEG electron microscope. Polymeric nanoparticle samples were prepared in 1 × PBS and diluted to 2 mg/mL with MilliQ water. 2 µL sample solution was applied to electron microscopy grid (Agar Scientific) with holey carbon film. Sample grids were blotted, and flash vitrified in liquid ethane using an automatic plunge freezingapparatus (Vitrbot) to control humidity (100%) and temperature (20° C.). Analysis was performed at -170° C. using the Gatan 626 cry-specimen holder (120,000× magnification; -5 µm defocus). Digital images were recorded on an in-line Eagle CCD camera and processed by ImageJ.

### Measurement of Critical Micelle Concentration

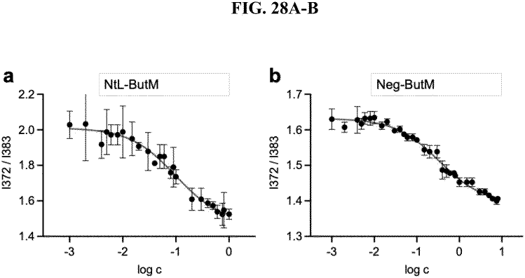

The critical micelle concentrations of NtL-ButM and Neg-ButM were determined by a fluorescence spectroscopic method using pyrene as a hydrophobic fluorescent probe (Refs. B30, B52; incorporated by reference in their entireties). A series of polymersolutions with concentration ranging from 1.0 × 10⁻⁴ to 2.0 mg mL⁻¹ were mixed with pyrene solution with a concentration of 1.2 ×10⁻³ mg mL⁻¹. The emission spectra of samples were recorded on a fluorescence spectrophotometer (HORIBA Fluorolog-3) at 20° C. using 335 nm as excitation wavelength. The ratio between the first (372 nm) and the third (383 nm) vibronic band of pyrene was used to plot against the concentration of the polymer. The data were processed on Prism software and fitted using Sigmoidal model (FIG. 28).

### Small Angle X-Ray Scattering Analysis of Micelles

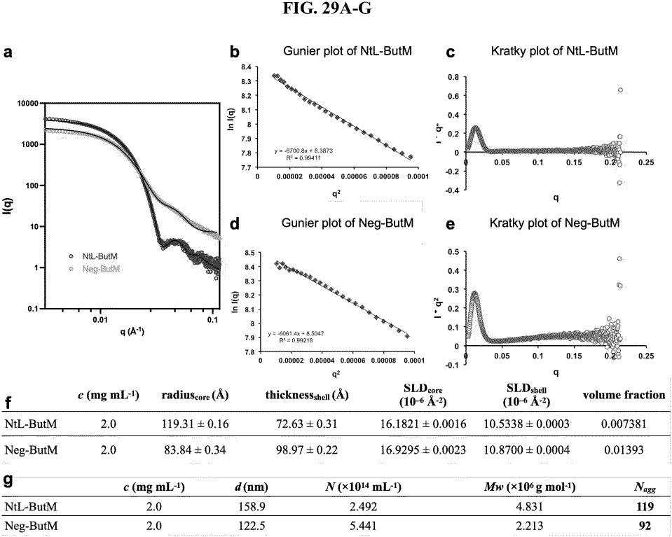

SAXS samples were made in 1 × PBS and filtered through 0.2 µm filters. All samples were acquired at Stanford Synchrotron Radiation Lightsource, SLAC National Accelerator Laboratory. SAXS data were analyzed by Igor Pro 8 software (FIG. 29). To acquire radius of gyration (Rg), data were plotted as ln(intensity) vs. q² at low q range. Then Rg were calculated from the slope of the linear fitting as shown in the equation (1).

$$l n \left(I \left(q\right)\right) = l n \left(I \left(0\right)\right) - \frac{{q}^{2} {R}_{g}^{2}}{3}$$

Kratky plot of the data were plotted from I q² vs. q to show the structure of the particles. Moreover,the data were fitted using polydispersed core-shell sphere model (FIGS. 29f,g)³¹. From the fitting, the radius of the core, thickness of the shell, and volume fraction of the micelle were derived andused to calculate the molecular weight of micelle and the mean distance between micelles usingflowing equations:

$$N = \frac{{ϕ}_{m i c e l l e}}{{v}_{m i c e l l e}}$$

$${M}_{w} = \frac{c {N}_{A}}{N}$$

$$d = {N}^{- 1 / 3} \times {10}^{7}$$

where N is the number of micelles per unit volume. ϕmicelle is the volume fraction of micelles derivedfrom fitting. vmicelle is the volume of a single micelle, which is calculated from 4/3 πR³, where R is the sum of radius of core and thickness of shell. Mw is the molecular weight of micelle, c is the polymer concentration. NA is Avogadro constant. d is the mean distance between the micelles inthe unit of nm. The aggregation number of micelles were calculated from dividing the molecular weight of micelle by the molecular weight of polymer.

### Mice

C3H/HeN and C3H/HeJ mice were maintained in a Helicobacter, Pasteurella and murine norovirus free, specific pathogen-free (SPF) facility at the University of Chicago. Breeding pairs of C3H/HeJ mice were originally purchased from the Jackson Laboratory. Breeding pairs of C3H/HeN mice were transferred from the germ-free (GF) facility. All experimental mice were bredin house and weaned at 3 weeks of age onto a plant-based mouse chow (Purina Lab Diet 5K67®)and autoclaved sterile water. Mice were maintained on a 12 h light/dark cycle at a roomtemperature of 20-24° C. GF C3H/HeN or C57BL/6 mice were bred and housed in the GnotobioticResearch Animal Facility (GRAF) at the University of Chicago. GF mice were maintained in Trexler-style flexible film isolator housing units (Class Biologically Clean) with Ancare polycarbonate mouse cages (catalog number N10HT) and Teklad Pine Shavings (7088; sterilizedby autoclave) on a 12 h light/dark cycle at a room temperature of 20-24° C. All experiments werelittermate controlled. All protocols used in this study were approved by the Institutional Animal Care and Use Committee of the University of Chicago. The FITC-dextran intestinal permeability assay in DSS treated mice was performed by Inotiv (Boulder, CO); SPF C57BL/6 mice were obtained from Taconic and housed in the Inotiv animal facility. The study was conducted in accordance with The Guide for the Care & Use of Laboratory Animals (8th Edition) and thereforein accordance with all Inotiv IACUC approved policies and procedures.

### Biodistribution Study Using In Vivo Imaging System (IVIS)

SPF C3H/HeJ mice were used for biodistribution studies. Azide labeled pHPMA-b-pBMA or pMAA-b-pBMA polymer was reacted with IR 750-DBCO (Thermo Fisher) and purified by hexaneprecipitation. After formulation into micelles, the fluorescently labeled NtL-ButM, or Neg-ButM wasadministered to mice by i.g. gavage. After 1 hr, 3 hr, 6 hr, or 24 hr, mice were euthanized, the major organs were collected from the mice and whole-organ fluorescence was measured via an IVIS Spectrum in vivo imaging system (Perkin Elmer). Images were processed and analyzed by Living Imaging 4.5.5 (Perkin Elmer).

### Butyrate Derivatization and Quantification Using LC-UV or LC-MS/MS

Simulated gastric fluid and simulated intestinal fluid (Fisher Scientific) were used for in vitro release analysis as described previously (Refs. B53-B54; incorporated by reference in their entireties). NtL-ButM or Neg-ButM were added to simulated gastric fluid or simulated intestinal fluid at a final concentration of 2 mg/mL at 37° C. At pre- determined time points, 20 µL of the solution was transferred into 500 µL of water: acetonitrile 1:1v/v. The sample was centrifuged using Amicon Ultra filters (Merck, 3 kDa molecular mass cutoff)at 13,000 × g for 15 min to remove polymers. The filtrate was stored at -80° C. before derivatization. For the in vivo release study in mouse GI tract, NtL-ButM or Neg-ButM micelle solutions were i.g.administered to SPF C3H/HeJ mice at 0.8 mg per g of body weight. Mice were euthanized at 1 hr, 2 hr, 4 hr, 8 hr, 12 hr, and 24 hr after the gavage. Luminal contents from the ileum, cecum, orcolon were collected in an EP tube. After adding 500 µL of 1 × PBS, the mixture was vortexed and sonicated for 10 min, and then centrifuged at 13,000 × g for 10 min. The supernatant was transferred and filtered through 0.45 m filter. The filtered solution was stored at -80° C. before derivatization.

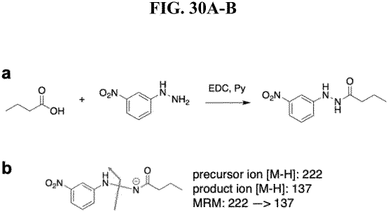

Sample derivatization (FIG. 30a): Samples were prepared and derivatized as described in the literature (Ref. B32; incorporated by reference in its entirety). 3-nitrophenylhydrazine (NPH) stock solution was prepared at 0.02 M in water:acetonitrile 1:1 v/v. EDC stock solution was prepared at 0.25 M in water:acetonitrile 1:1 v/v. 4-methylvaleric acid was added as internal standard. Samples were mixed with NPH stock and EDC stock at 1:1:1 ratio by volume. The mixture was heated by heating block at 60° C. for 30 min. Samples were filtered through 0.22 µm filters and transferred into HPLC vials and stored at 4° C. before analysis.

LC conditions: The instrument used for quantification of butyrate was Agilent 1290 UHPLC. Column: ThermoScientific C18 4.6 × 50 mm, 1.8 µm particle size, at room temperature. Mobile phase A: water with 0.1% v/v formic acid. Mobile phase B: acetonitrile with 0.1% v/v formic acid. Injection volume: 5.0 µL. Flow rate: 0.5 mL/min. Gradient of solvent: 15% mobile phase B at 0.0 min; 100% mobile phase B at 3.5 min; 100% mobile phase B at 6.0 min; 15% mobile phase B at 6.5 min.

ESI-MS/MS method: The instrument used to detect butyrate was an Agilent 6460 Triple Quad MS-MS. Both derivatized butyrate-NPH and 4-methylvaleric-NPH were detected in negative mode. The MS conditions were optimized on pure butyrate-NPH or 4-methylvaleric-NPH at 1 mM.The fragment voltage was 135 V and collision energy was set to 18 V. Multiple reaction monitoring(MRM) of 222 → 137 was assigned to butyrate (FIG. 30b), and MRM of 250 → 137 was assignedto 4-methylvaleric acid as internal standard. The ratio between MRM of butyrate and 4- methylvaleric acid was used to quantify the concentration of butyrate.

### RNA Sequencing and Data Analysis

Starting at the time of weaning, GF C3H/HeN mice were i.g. administered with PBS, NtL-ButM, or control polymer at 0.8 mg/g of body weight once daily for one week. After that time, mice wereeuthanized, and the ileum tissue was collected and washed thoroughly. The ileal epithelial cells (IECs) were separated from intestinal tissue by inverting ileal tissue in 0.30 mM EDTA, incubatingon ice for 30 min with agitation every 5 min. RNA was extracted from the IECs using an RNA isolation kit (Thermo Fisher Scientific) according to manufacturer’s instruction. RNA samples were submitted to the University of Chicago Functional Genomics Core for library preparation and sequencing on a HiSeq2500 instrument (Illumina, Inc.). 50 bp single-end (SE) reads weregenerated. The quality of raw sequencing reads was assessed by FastQC (v0.11.5). Transcript abundance was quantified by Kallisto (v0.45.0) with Gencode gene annotation (release M18, GRCm38.p6), summarized to gene level by tximport (v1.12.3), Trimmed Mean of M-values (TMM) normalized, and log2 transformed. Lowly expressed genes were removed (defined as, counts permillion reads mapped [CPM] <3). Differentially expressed genes (DEGs) between groups of interest were detected using limma voom with precision weights (v3.40.6) (Ref. B55; incorporated by reference in its entirety). Experimental batch and gender were included as covariates for the model fitting. Significance level and fold changeswere computed using empirical Bayes moderated t-statistics test implemented in limma. Significant DEGs were filtered by FDR-adjusted P<0.05 and fold change ≥ 1.5 or ≤ -1.5. A morestringent P-value cutoff (e.g., FDR-adjusted P<0.005) may be used for visualization of a select number of genes on expression heatmaps.

### Intelectin Stain and Microscope Imaging

GF C57BL/6 mice were i.g. administered NtL-ButM at 0.8 mg/g of body weight or PBS once dailyfor one week beginning at weaning. After that time, the mice were euthanized and perfused, smallintestine tissue was obtained, rolled into Swiss-rolls, and prepared into tissue section slides. Thetissue section slides were fixed and stained with fluorescent anti-intelectin antibody (R&DSystems, Clone 746420) and DAPI (ProLong antifade reagent with DAPI). The slides were imaged using a Leica fluorescence microscope. Images were processed by ImageJ software anddata were plotted and analyzed by Prism software.

### In Vivo FITC-Dextran Permeability Assay

SPF C57BL/6 8-10 wks old female mice were treated with 2.5% DSS in their drinking water for 7 days. The mice received intragastric administration twice daily, at approximately 10-12 hrintervals, of either PBS, or ButM (800, 400 or 200 mg/kg), or once daily with CsA at 75 mg/kg as the positive treatment control. On day 7, DSS was removed from the drinking water for the remainder of the study. On day 10, mice were fasted for 3 hr and dosed with 0.1 mL of FITC- dextran 4 kDa (at 100 mg/mL). 4 hr post dose mice were anesthetized with isoflurane and bled toexsanguination followed by cervical dislocation. The concentration of FITC in the serum was determined by spectrofluorometry using as standard serially diluted FITC-dextran. Serum from mice not administered FITC-dextran was used to determine the background. A similar permeability assay was also performed in the antibiotic-depletion model as previously described (Ref. B19; incorporated by reference in its entirety). Littermate-controlled SPF C57BL/6 mice at 2 wks of age were gavaged daily with a mixture of antibiotics (0.4 mg kanamycin sulfate, 0.035 mg gentamycin sulfate, 850 U colistin sulfate, 0.215 mg metronidazole, and 0.045 mg vancomycin hydrochloride in 100 µL PBS) for 7 days until weaning. At weaning, mice were then treated with either PBS or ButM (0.8 mg/g) twicedaily for 7 days. After the final treatment, the mice were fasted for 3 hr. and dosed with 50 mg/kg body weight of FITC-dextran 4 kDa (at 50 mg/mL). Blood was collected at 1.5 hr. post- administration via cheek bleed and the concentration of FITC in the serum was measured as described above.

### Peanut Sensitization, ButM Treatment and Challenge

SPF C3H/HeN mice were treated with 0.45 mg of vancomycin in 0.1 mL by intragastric gavage for 7 days pre-weaning and then with 200 mg/L vancomycin in the drinking water throughout theremainder of the sensitization protocol. Age- and sex-matched 3-wk-old littermates were sensitized weekly by intragastric gavage with defatted, in-house made peanut extract prepared from unsalted roasted peanuts (Hampton Farms, Severn, NC) and cholera toxin (CT) (List Biologicals, Campbell, CA) as previously described (Refs. B19, B39; incorporated by reference in their entireties). Sensitization began at weaning and continued for 4 weeks. Prior to each sensitization the mice were fasted for 4-5 hr and then given200 µl of 0.2 M sodium bicarbonate to neutralize stomach acids. 30 min later the mice received 6 mg of peanut extract and 10 µg of cholera toxin (CT) in 150 µl of PBS by intragastric gavage.

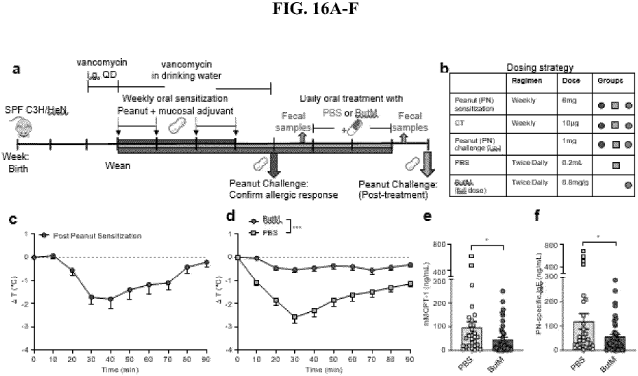

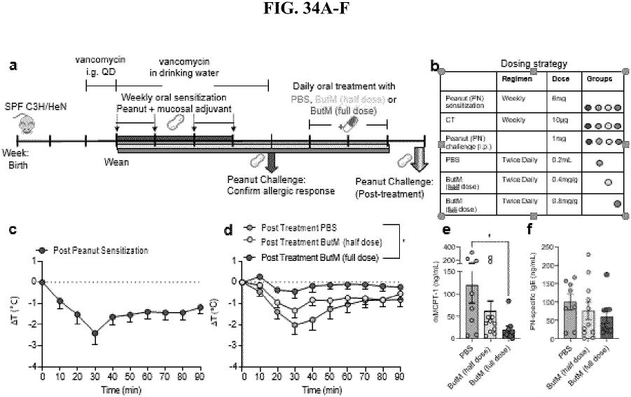

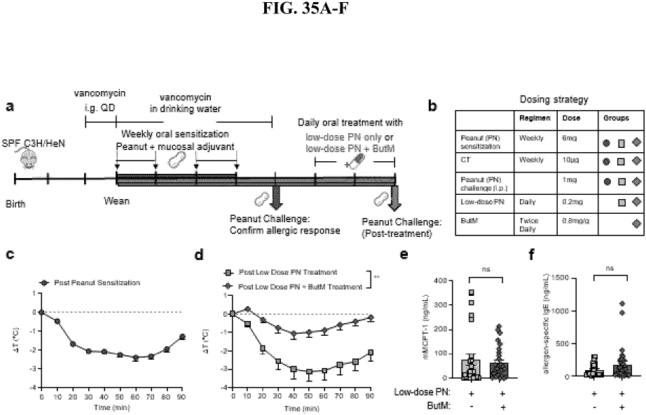

After 4 weeks of sensitization, mice were permitted to rest for 1 wk before a subset of mice was challenged by intraperitoneal (i.p.) administration of 1 mg peanut extract in 200 µl of PBS to confirm that the sensitization protocol induced a uniform allergic response. Rectal temperature was measured immediately following challenge every 10 minutes for up to 90 min using an intrarectal probe, and the change in core body temperature of each mouse was recorded. The remaining mice were not challenged and were randomly assigned into experimental groups. In the monotherapy experiment (FIG. 16), one group of mice was treated with ButM twice daily by intragastric gavage at 0.8 mg of total polymer per gram of mouse body weight (0.8 mg/g) for twoweeks, and another group of mice received PBS. In the dose-dependent study (FIG. 34), mice were treated with either PBS, ButM at 0.8 mg/g (full dose), or ButM at 0.4 mg/g (half dose) twicedaily. Additionally, in the experiment where ButM was delivered synchronously with low dose exposure to allergen (FIG. 35), one group of mice was treated daily for two weeks with low dose(200 µg) of peanut powder (PB2™ (PB2 Foods, Tifton, GA), and another group of mice receivedboth PB2™ (200 µg) daily and ButM at 0.8 mg/g twice daily. After the treatment window, mice were challenged with i.p. administration of 1 mg peanut extract and core body temperature was measured for 90 min. Serum was collected from mice 90 minutes after challenge for measurementof mMCPT-1 and additionally at 24 hr after challenge for measurement of peanut-specific IgE. Collected blood was incubated at room temperature for 1 hour and centrifuged for 7 minutes at 12,000 g at room temperature, and sera were collected and stored at ⁻80° C. before analysis. Serum antibodies and mMCPT-1 were measured by ELISA.

### Measurement of Mouse Mast Cell Protease 1 (mMCPT-1) and Serum Peanut-Specific IgE Antibodies Using ELISA

mMCPT-1 was detected using the MCPT-1 mouse uncoated ELISA kit (ThermoFisher) followingthe protocol provided by manufacturer. For the peanut-specific IgE ELISA, sera from individual mice were added to peanut coated Maxisorp Immunoplates (Nalge Nunc International, Naperville, IL). Peanut-specific IgE Abs were detected with goat anti-mouse IgE-unlabeled (SouthernBiotechnology Associates, Birmingham, AL) and rabbit anti-goat IgG-alkaline phosphatase (Invitrogen, Eugene, Oregon) and developed with p-nitrophenyl phosphate “PNNP” (SeraCare Life Sciences, Inc. Milford, MA). OD values were converted to nanograms per milliliter of IgE by comparison with standard curves of purified IgE by linear regression analysis and are expressedas the mean concentration for each group of mice ± s.e.m. Statistical differences in serum Ab levels were determined using a two-tailed Student’s t test. A P value < 0.05 was considered significant.

### 16S rRNA Targeted Sequencing

Bacterial DNA was extracted using the QIAamp PowerFecal Pro DNA kit (Qiagen). The V4-V5 hypervariable region of the 16S rRNA gene from the purified DNA was amplified using universal bacterial primers - 563F (5′-nnnnnnnn-NNNNNNNNNNNN-AYTGGGYDTAAA-GNG-3′) and 926R (5′-nnnnnnnn-NNNNNNNNNNNN-CCGTCAATTYHT- TTRAGT-3′), where ‘N’ represents the barcodes, ‘n’ are additional nucleotides added to offset primer sequencing. Illumina sequencing-compatible Unique Dual Index (UDI) adapters were ligated onto pools using the QIAsep 1-step amplicon library kit (Qiagen). Library QC was performed using Qubit and Tapestation before sequencing on an Illumina MiSeq platform at the Duchossois Family InstituteMicrobiome Metagenomics Facility at the University of Chicago. This platform generates forwardand reverse reads of 250 bp which were analyzed for amplicon sequence variants (ASVs) using the Divisive Amplicon Denoising Algorithm (DADA2 v1.14) structure (Ref. B56; incorporated by reference in its entirety).. Taxonomy was assigned to the resulting ASVs using the Ribosomal Database Project (RDP) database with a minimum bootstrapscore of 50 (Ref. B57; incorporated by reference in its entirety). The ASV tables, taxonomic classification, and sample metadata were compiled using the phyloseq data structure (Ref. B58; incorporated by reference in its entirety). Subsequent 16S rRNA relative abundance analyses and visualizations were performed using R version 4.1.1 (R Development Core Team, Vienna, Austria).

### Microbiome Analysis

To identify changes in the microbiome across conditions, a linear discriminant analysis effect size(LEfSe) analysis was performed in R using the microbiomeMarker package and the run_lefse function (Refs. B59-B60; incorporated by reference in their entireties). Features, specifically taxa, can be associated with or without a given condition (e.g.,ButM post-treatment vs PBS post-treatment) and an effect size can be ascribed to that differencein taxa at a selected taxonomic level (LDA score). For the LEfSe analysis, genera were comparedas the main group, a significance level of 0.05 was chosen for both the Kruskall-Wallis and Wilcoxon tests and a linear discriminant analysis cutoff of 1.0 was implemented. The abundanceof Clostridium Cluster XIVA in post-treatment samples was also determined by quantitative PCR(qPCR) using the same DNA analyzed by 16S rRNA targeted sequencing. Commonly used primers 8F⁶¹ and 338R⁶² were used to quantify total copies of the 16S rRNA gene for normalization purposes. Primers specific for Clostridium Cluster XIVa⁶³ were validated by PCR and qPCR. Primer sequences are listed in Table 1. qPCR was performed usingPowerUp SYBR green master mix (Applied Bioystems) according to manufacturer’s instructions. The abundance of Clostridium Cluster XIVa is calculated by 2^{-CT}, multiplied by a constant to bringall values above 1 (1 x 10¹⁶), and expressed as a ratio to total copies 16S per gram of raw fecal content.

TABLE 1

Primer sequences for qPCR.

| Primers to quantify total bacterial load | Primer sequence (5′->3′) | Reference |
|---|---|---|
| | 8F: 5′AGAGTTTGATCCTGGCTCAG | Ref. 61; Turner S et al, 1999 |
| | 338R: 5′-TGCTGCCTGCCGTAGGAGT | Ref. 62; Amann RI et al, 1995 |
| Target | Primer sequence (5′>3′) | Reference |
| Clostridium Cluster XIVa | Forward: 5′-AAATGACGGTACCTGACTAA-3′ | Ref. 63; Matsuki T et al, 2002 |
| | Reverse: 5′-CTTTGAGTTTCATTCTTGCGAA-3′ | |

### Toxicity Study

The toxicity effect of pHPMA-b-pBMA on SPF C3H/HeJ mice was measured by hematological analysis on Vet Axcel Chemistry Analyzer. Mice were treated with NtL-ButM at 0.8 mg/g of bodyweight daily by intragastric gavage for 6 weeks. Every week, a blood sample of each mouse wasobtained and analyzed by the chemistry analyzer according to manufacturer’s instruction.

### II. Results

### Copolymers Formulate Butyrate Into Water-Suspensible Micelles

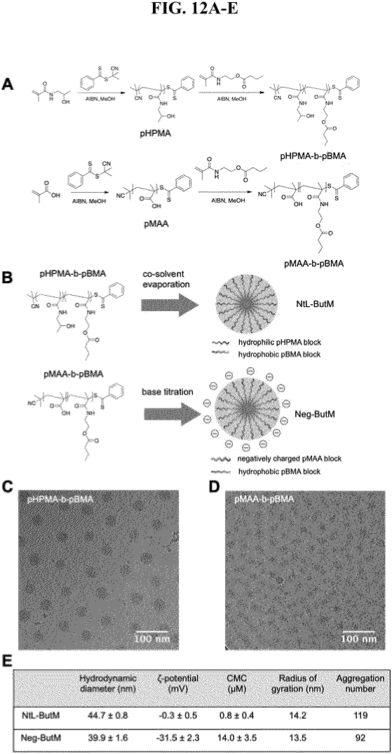

The block copolymer amphiphile pHPMA-b-pBMA was synthesized through two steps of reversible addition-fragmentation chain-transfer (RAFT) polymerization (FIG. 12a). The hydrophilic block was formed from N-(2-hydroxypropyl) methacrylamide (HPMA), while the hydrophobic block was from N-(2-butanoyloxyethyl) methacrylamide (BMA), thus connecting a backbone sidechain to butyrate with an ester bond. This ester bond can be hydrolyzed in the presence of esterase and releases butyrate in the GI tract, resulting in a water-soluble polymer as a final product. In addition to pHPMA-b-pBMA, we also synthesized pMAA-b-pBMA, which has an anionic hydrophilic block formed from methacrylic acid (MAA) (FIG. 12a). At the block size ratios used herein, both pHPMA-b-pBMA and pMAA-b-pBMA contain 28% of butyrate by weight.

These block copolymers can be then formulated into nanoscale micelles to achieve high suspensibility in aqueous solutions as well as controlled release of butyrate from the core. The pHPMA-b-pBMA was self-assembled into neutral micelles (NtL-ButM) through a cosolvent evaporation method (FIG. 12b). The hydrophobic pBMA block forms the core, while the hydrophilic pHPMA forms the corona. In contrast, pMAA-b-pBMA cannot be formulated into micelles by this method because of the formation of intramolecular hydrogen bonds between pMAA chains (Ref. B26; incorporated by reference in its entirety). Such bonding can, however, be disrupted when a strong base, here NaOH, is titrated into the mixture of pMAA-b-pBMA polymer to change methacrylic acid into ionized methacrylate (Refs. B27-B29; incorporated by reference in their entireties). Upon base titration, pMAA-b-pBMA polymer can then self-assemble into negatively charged micelles (Neg-ButM) (FIG. 12b). Cryogenic electron microscopy (CryoEM) revealed the detailed structure of the micelles, especially the core structure made of pBMA, which was more condensed with higher contrast. CryoEM images indicated that the diameter of the core of NtL-ButM was 30 nm, while Neg-ButM had a smaller core diameter of 15 nm (FIGS. 12c, d). Both NtL-ButM and Neg-ButM have similar sizes of 44.7 ± 0.8 nm and 39.9 ± 1.6 nm, respectively, measured by dynamic light scattering (DLS) (FIG. 12e). Their low polydispersity index below 0.1 indicated the monodispersity of those micelles. NtL-ButM has a near-zero ç-potential of -0.3 ± 0.5 mV, while Neg-ButM’s is -31.5 ± 2.3 mV due to the ionization of methacrylic acid (FIG. 12e).To obtain the critical micelle concentration (CMC) of NtL-ButM and Neg-ButM, which indicates the likelihood of formation and dissociation of micelles in aqueous solutions, pyrene was added during the formulation and the fluorescence intensity ratio between the first and third vibronic bands of pyrene was plotted to calculate the CMC. Results showed that Neg-ButM had a higher CMC of 14.0 ± 3.5 µM, compared to the CMC of NtL-ButM, which was 0.8 ± 0.3 µM (FIG. 12e). The higher CMC indicated that Neg-ButM micelles would be easier to dissociate in solution, possibly because the surface charge made the micellar structure less stable compared to the neutral micelle NtL-ButM. In addition, we conducted small angle X-ray scattering (SAXS) analysis on both micelles to obtain the aggregation number (FIG. 29). As indicated from Guinier plots, radii of gyration for NtL-ButM and Neg-ButM were 14.2 nm and 13.5 nm (FIG. 12e), respectively, and the structures of micelles were confirmed to be spheres from Kratky plots of SAXS data (FIG. 29). The SAXS data was then fitted with a polydispersity core-shell sphere model with the assumptions that the micelle has a spherical core with a higher scattering length densities (SLD) and a shell with a lower SLD (Ref. B31; incorporated by reference in its entirety). The model provided the volume fraction of the micelles, the radius of the core, and the thickness of the shell, allowing calculation of the aggregation number and mean distance between micelles. According to the fitting results, aggregation numbers for NtL- ButM and Neg-ButM were 119 and 92, respectively (FIG. 12e).

### Butyrate Micelles Release Butyrate in the Lower GI Tract

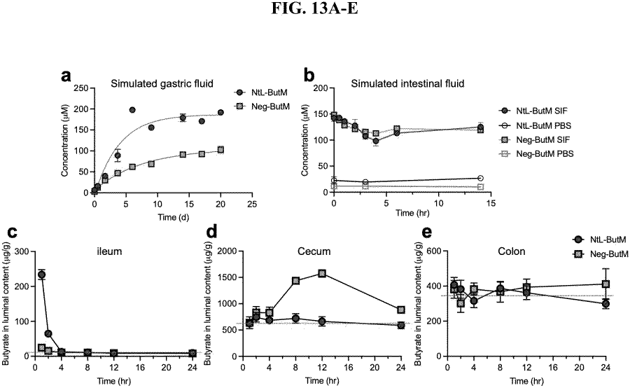

Given that butyrate is linked to the micelle-forming chain via ester bonds, the release of butyrate was validated in ex vivo conditions, including in simulated gastric fluid and simulated intestinal fluid that mimic those biological environments. In the simulated gastric fluid, both Neg-ButM and NtL- ButM showed negligible release of butyrate within hours, and sustained slow release over 3 weeks, while Neg-ButM had even slower release rate than NtL-ButM (FIG. 13a). The anionic surface of Neg-ButM in the acidic environment is likely responsible for the resistance to the hydrolysis of the BMA core. By contrast, in simulated intestinal fluid, both micelles released the most of their butyrate within minutes in the presence of a high concentration of the esterase pancreatin (FIG. 13b).

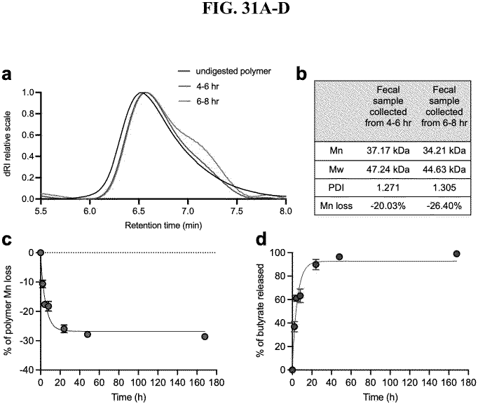

Butyrate levels were measured in the mouse GI tract after administering a single dose of NtL- ButM or Neg-ButM by intragastric gavage (i.g.). Both LC-UV and LC-MS/MS methods have been used to measure butyrate concentrations in the luminal contents of the ileum, cecum, and colon, the sites where butyrate producing bacteria normally reside (Refs. B32-B33; incorporated by reference in their entireties). However, because the baseline concentration in the ileum was too low for the UV detector, LC-MS/MS was used to measure the butyrate concentration in that GI tract segment. NtL-ButM dramatically increased the butyrate concentration in the ileum for up to 2 hr after gavage (FIG. 13c), but this was short lived, and the butyrate concentration did not increase in either the cecum or colon (FIG. 13d, e). Neg-ButM raised butyrate concentrations by 3-fold in the cecum starting from 4 hr after gavage and lasting for at least another 8 hr but not in the ileum or colon (FIGS. 13c-e). It is possible that the butyrate released in the cecum will continuously flow into the colon; inability to detect increased concentrations of butyrate in the colon is likely due to its rapid absorption and metabolism by the colonic epithelium. In addition, the polymer backbone of the micelles remained intact when passing through the GI tract. Less than 28% molecular weight loss was observed — the percentage of butyrate content — of the polymer in fecal samples collected from 4-8 hr after oral administration (FIG. 31a, b). Moreover, when incubated in a hydrolytic environment in vitro, the polymer backbone remained intact after releasing most of the butyrate over 7 days in 125 mM sodium hydroxide solution.

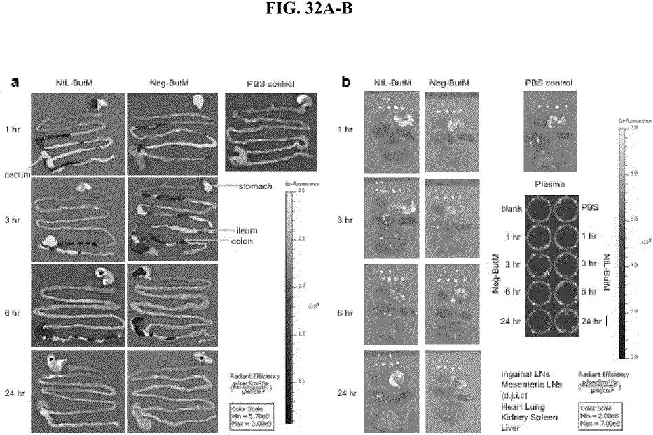

Transit of the micelles through the GI tract was monitored by administering fluorescently labeled NtL-ButM or Neg-ButM to mice i.g. and visualizing their biodistribution via an In Vivo Imaging System (IVIS) (FIG. 32). The fluorescent marker was conjugated to the polymer chain, allowing visualization of the transit of the polymer backbone itself. The IVIS results validated that the polymeric micelles were retained in the mouse GI tract for more than 6 hr. after gavage. The neutral micelle NtL-ButM passed through the stomach and small intestine within 2 hr. and accumulated in the cecum. However, negatively charged Neg-ButM accumulated in the stomach first and then gradually traveled through the small intestine to the cecum. Overall, Neg-ButM had a longer retention time in the stomach and small intestine, which is possibly due to the stronger adhesive effect to the gut mucosa (Refs. B26, B34-B35; incorporated by reference in their entireties). Both micelles were cleared from the GI tract within 24 hr. after administration. In addition, the fluorescence signal was measured in other major organs and plasma by IVIS (FIG. 32b), as well as the butyrate concentration in the plasma by LC-MS/MS. The signals were all below the detection limit from both methods, indicating that there was negligible absorption of these butyrate micelles into the blood circulation from the intestine, consistent with the desire to deliver butyrate to the lower GI tract and to avoid any complexities of systemic absorption of the polymer or micelles.

### Ileum-Targeting Butyrate Micelles Up-Regulate AMP Genes in the Ileal Epithelium

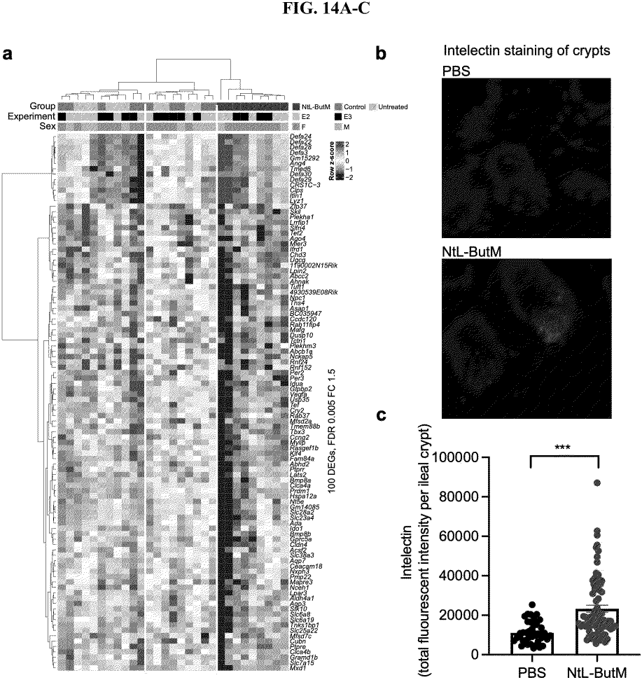

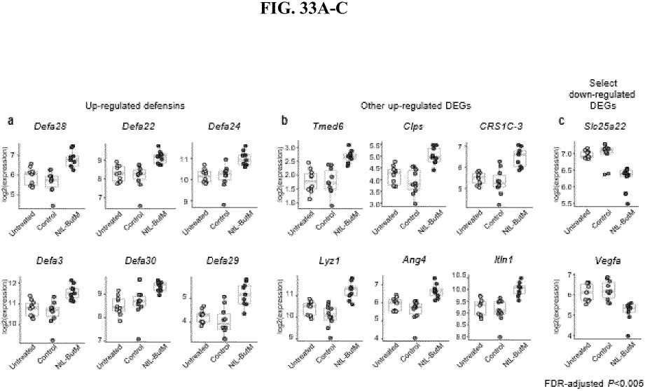

Delivery of butyrate to the lower GI tract could affect the host immune response by interacting with the intestinal epithelium. To investigate whether and how our butyrate micelles regulate gene expression in the distal small intestine, RNA sequencing of the ileal epithelial cell compartment was performed (FIG. 14a). Germ-free (and thus butyrate-depleted) C3H/HeN mice were treated daily with NtL-ButM i.g. for one week and ileal epithelial cells were collected for RNA isolation and sequencing. Because only NtL-ButM (and not Neg-ButM) released butyrate in the ileum, only NtL- ButM was used for this experiment to examine local effects. NtL-ButM-treated mice had unique gene expression signatures compared to those treated with PBS or control polymer, which consists of the same polymeric structure but does not contain butyrate. Such differences showed no dependence on sex. Most genes upregulated by NtL-ButM treatment were Paneth cell derived antimicrobial peptides (AMPs), including angiogenin 4 (Ang4), lysozyme-1 (Lyz1), intelectin (Itln1) and several defensins (Defa3, Defa22, Defa24 etc.) (FIG. 14a, FIG. 33). The protein level of intelectin, one of the up-regulated AMPs, was quantified (FIG. 14b, c). Intelectin is known to be expressed by Paneth cells which reside in small intestinal crypts and can recognize the carbohydrate chains of the bacterial cell wall (Ref. B36; incorporated by reference in its entirety). Paneth cell AMPs have largely been characterized in C57BL/6 mice and specific reagents are available for their detection in that strain (Ref. B37; incorporated by reference in its entirety). GF C57BL/6 mice were gavaged daily with NtL-ButM or PBS for one week. Immunofluorescence microscopy of ileal sections revealed that the NtL-ButM treated group expressed a large amount of intelectin in the crypts of the ileal tissue. However, images from the PBS group showed limited intelectin signal (FIG. 14b). Quantification using ImageJ of relative fluorescence intensity per ileal crypt also showed that the NtL-ButM group had significantly higher expression of intelectin compared to the PBS control (FIG. 14c). The intelectin staining thus further supported the pharmacological effects of NtL-ButM; up-regulation of intelectin induced by NtL- ButM was not only demonstrated on the transcriptional level by RNAseq but was also validated at the protein level.

### Butyrate Micelles Repair Intestinal Barrier Function

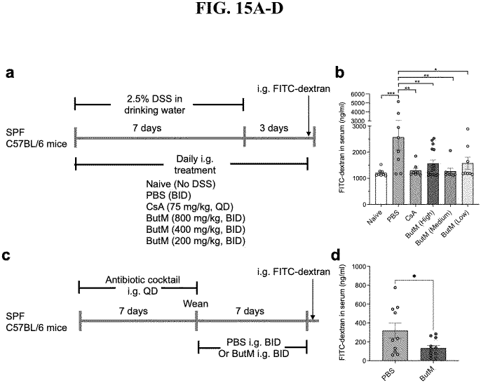

Butyrate-producing bacteria play an important role in the maintenance of the intestinal barrier. To assess the effects of locally-delivered butyrate on intestinal barrier integrity, mice were treated with the chemical perturbant DSS for 7 days to induce epithelial barrier dysfunction (Ref. B38; incorporated by reference in its entirety). Due to the different biodistribution and butyrate release behaviors in vivo from the two butyrate micelles, it was reasoned that the combined dosing of NtL-ButM and Neg-ButM would cover the longest section of the lower GI tract and last for a longer time; thus, a 1:1 combination of NtL-ButM and Neg-ButM (abbreviated as ButM) was selected for study. Throughout DSS treatment, and for three days after DSS administration was terminated, mice were orally gavaged twice daily with either PBS or ButM at three different concentrations, or once daily with cyclosporin A (CsA) as the positive therapeutic control (as outlined in FIG. 15a). Intragastric gavage of 4 kDa FITC-dextran was used to evaluate intestinal barrier permeability. A significantly higher concentration of FITC-dextran was detected in the serum of DSS-treated mice gavaged only with PBS, demonstrating an impaired intestinal barrier. Naive mice (without DSS exposure), or the DSS- treated mice that also received either CsA or ButM at all three concentrations had similar serum levels of FITC-dextran, indicating that treatment with ButM successfully repaired the DSS- induced injury to the barrier (FIG. 15b). Additionally, neonatal antibiotic treatment impairs homeostatic epithelial barrier function and increases permeability to food antigens (ref. B19; incorporated by reference in its entirety). Thus, it was further evaluated whether the ButM treatment can reduce intestinal barrier permeability in antibiotic-treated mice (FIG. 15c). Similar to what was observed in the DSS-induced model, the mice treated with ButM had significantly lower FITC-dextran levels in the serum compared to mice that received PBS (FIG. 15d), demonstrating that ButM effectively rescued both DSS-induced and antibiotic-induced intestinal barrier dysfunction.

### Butyrate Micelles Ameliorated Anaphylactic Responses in Peanut Allergic Mice

To evaluate the efficacy of the butyrate-containing micelles in treating food allergy, ButM was tested in a well-established murine model of peanut-induced anaphylaxis (Refs. B19, B39; incorporated by reference in their entireties). All of the mice were treated with vancomycin to induce dysbiosis. Beginning at weaning, vancomycin-treated SPF C3H/HeN mice were intragastrically sensitized weekly for 4 weeks with peanut extract (PN) plus the mucosal adjuvant cholera toxin (CT) (FIGS. 16a, b), as previously described (Refs. B19, B39; incorporated by reference in their entireties). Following sensitization, some of the mice were challenged with intraperitoneal (i.p.) PN and their change in core body temperature was monitored to ensure that the mice were uniformly sensitized; a decrease in core body temperature is indicative of anaphylaxis (FIG. 16c). The rest of the sensitized mice were then treated i.g. twice daily for 2 weeks with either PBS or the combined micelle formulation ButM. After 2 weeks of therapy, the mice were challenged by i.p. injection of PN and their core body temperature was assessed to evaluate the response to allergen challenge. Compared with PBS-treated mice, allergic mice that were treated with ButM experienced a significantly reduced anaphylactic drop in core body temperature (FIG. 16d). In addition, ButM- treated mice also had significantly reduced concentrations of mouse mast cell protease-1 (mMCPT-1) and peanut-specific IgE detected in the serum (FIGS. 16e, f). mMCPT-1 is a chmyase expressed by intestinal mucosal mast cells; elevated concentrations of mMCPT-1 increase intestinal barrier permeability during allergic hypersensitivity responses (Refs. B40-B41: incorporated by reference in their entireties). Furthermore, these effects of ButM on the peanut allergic mice were dose-dependent, as we observed that reducing the dose of ButM by half was not as effective as the full dose in protecting mice from an anaphylactic response. Together, these results demonstrate that ButM as a monotherapy can effectively prevent allergic responses to food in sensitized mice.

Because OIT is the only FDA-approved treatment for peanut allergy, it was tested whether ButM would be an effective treatment when delivered synchronously with low dose exposure to allergen in sensitized mice (FIGS. 35a, b). Low-dose PN treatment alone in this regimen had no therapeutic effect, possibly due to insufficient length of treatment to achieve functional OIT, as low-dose PN-treated mice had a comparable drop in core body temperature to those that received no treatment (FIGS. 35c, d). However, mice treated with low-dose PN plus ButM exhibited a significantly reduced drop in core body temperature indicative of a substantially decreased anaphylactic response. This suggests a potential clinical use of butyrate micelles for patients undergoing OIT. However, the treatment did not reduce serum peanut-specific IgE, as has been observed in several clinical studies of OIT (Refs. B8, B43; incorporated by reference in their entireties) (FIG. 35f).

### Butyrate Micelles After Fecal Microbiota and Promote Recovery of Clostridia After Antibiotic Exposure

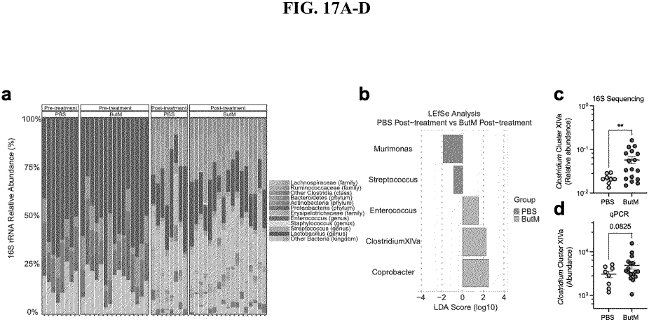

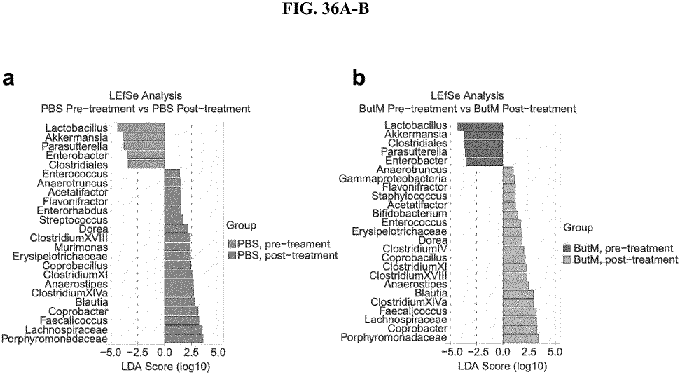

Given that ButM induces AMPs and may alter gut metabolism, it was examined whether treatment altered the fecal microbiome. In the mouse model of peanut allergy described above, dysbiosis was induced by treating mice with vancomycin one week before the start of allergen sensitization and throughout the sensitization regimen. Vancomycin depletes Gram positive bacteria, including Clostridial species (Ref. B43: incorporated by reference in its entirety). After sensitization, vancomycin was removed from the drinking water and the fecal microbial composition of the allergic mice was compared before and after treatment with PBS or ButM (see timepoints collected in FIG. 16a). 16S rRNA targeted sequencing confirmed depletion of Clostridia in vancomycin-treated mice; the fecal microbiota was instead dominated by Lactobacillus and Proteobacteria (FIG. 17a, left). After halting vancomycin administration, regrowth of Clostridia (including Lachnospiraceae and others) and Bacteroidetes was observed in both the PBS and ButM treated groups (FIG. 17a, right, FIG. 36). When comparing differentially abundant taxa between treatment groups by LEfSe analysis, Murimonas and Streptococcus were significantly higher in relative abundance in the PBS post-treatment group when compared to the ButM post-treatment group (FIG. 17b). ButM treatment significantly increased the relative abundance of Enterococcus, Coprobacter, and Clostridium Cluster XIVa (FIG. 17b). Clostridium Cluster XIVa is a numerically predominant group of bacteria (in both mice and humans) that is known to produce butyrate, modulate host immunity, and induce Tregs (Refs. B43-B44; incorporated by reference in their entireties). The relative abundance of Clostridium Cluster XIVa in mice treated with ButM was significantly increased in the 16S data set (FIG. 17c); the enriched abundance of this taxa was quantified by qPCR (FIG. 17d). The finding of increased abundance of Clostridium Cluster XIVA after treatment with ButM is in keeping with earlier work which showed that butyrate sensing by peroxisome proliferator-activated receptor (PPAR-γ) shunts colonocyte metabolism toward β-oxidation, creating a local hypoxic niche for these oxygen sensitive anaerobes (Ref. B45; incorporated by reference in its entirety).

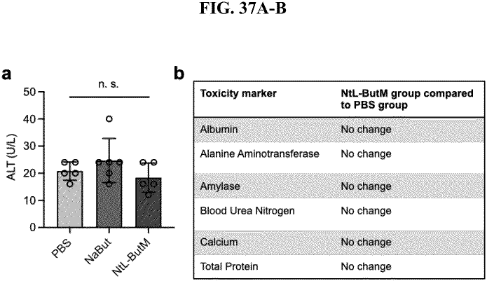

Characterization of toxicity of the butyrate micelles was conducted. It was demonstrated that treatment induced no changes among the serological toxicity markers tested, including serum albumin, alanine aminotransferase, amylase, blood urea nitrogen, calcium, and total protein, over a 6-week course of daily treatment (FIG. 37).

### REFERENCES

The following references, some of which are cited above, are herein incorporated by reference in their entireties.

A1. Chang, P. V.; Hao, L.; Offermanns, S.; Medzhitov, R., The microbial metabolite butyrate regulates intestinal macrophage function via histone deacetylase inhibition. Proceedings of the National Academy of Sciences 2014, 111 (6), 2247.

A2. Mathewson, N. D.; Jenq, R.; Mathew, A. V.; Koenigsknecht, M.; Hanash, A.; Toubai, T.; Oravecz-Wilson, K.; Wu, S.-R.; Sun, Y.; Rossi, C.; Fujiwara, H.; Byun, J.; Shono, Y.; Lindemans, C.; Calafiore, M.; Schmidt, T. M.; Honda, K.; Young, V. B.; Pennathur, S.; van den Brink, M.; Reddy, P., Gut microbiome-derived metabolites modulate intestinal epithelial cell damage and mitigate graft-versus-host disease. Nature Immunology 2016, 17 (5), 505-513.

A3. Fachi, J. L.; Felipe, J. S.; Pral, L. P.; da Silva, B. K.; Corrêa, R. O.; de Andrade, M. C. P.; da Fonseca, D. M.; Basso, P. J.; Câmara, N. O. S.; de Sales, E. S. É.; Dos Santos Martins, F.; Guima, S. E. S.; Thomas, A. M.; Setubal, J. C.; Magalhães, Y. T.; Forti, F. L.; Candreva, T.; Rodrigues, H. G.; de Jesus, M. B.; Consonni, S. R.; Farias, A. D. S.; Varga-Weisz, P.; Vinolo, M. A. R., Butyrate Protects Mice from Clostridium difficile-Induced Colitis through an HIF-1-Dependent Mechanism. Cell Rep 2019, 27 (3), 750-761.e7.

A4. Arpaia, N.; Campbell, C.; Fan, X.; Dikiy, S.; van der Veeken, J.; deRoos, P.; Liu, H.; Cross, J. R.; Pfeffer, K.; Coffer, P. J.; Rudensky, A. Y., Metabolites produced by commensal bacteria promote peripheral regulatory T-cell generation. Nature 2013, 504 (7480), 451-455.

A5. Furusawa, Y.; Obata, Y.; Fukuda, S.; Endo, T. A.; Nakato, G.; Takahashi, D.; Nakanishi, Y.; Uetake, C.; Kato, K.; Kato, T.; Takahashi, M.; Fukuda, N. N.; Murakami, S.; Miyauchi, E.; Hino, S.; Atarashi, K.; Onawa, S.; Fujimura, Y.; Lockett, T.; Clarke, J. M.; Topping, D. L.; Tomita, M.; Hori, S.; Ohara, O.; Morita, T.; Koseki, H.; Kikuchi, J.; Honda, K.; Hase, K.; Ohno, H., Commensal microbe-derived butyrate induces the differentiation of colonic regulatory T cells. Nature 2013, 504, 446.

A6. Smith, P. M.; Howitt, M. R.; Panikov, N.; Michaud, M.; Gallini, C. A.; Bohlooly-Y, M.; Glickman, J. N.; Garrett, W. S., The microbial metabolites, short-chain fatty acids, regulate colonic Treg cell homeostasis. Science (New York, N.Y.) 2013, 341 (6145), 569-573.

A7. Liu, H.; Wang, J.; He, T.; Becker, S.; Zhang, G.; Li, D.; Ma, X., Butyrate: A Double-Edged Sword for Health? Adv Nutr 2018, 9 (1), 21-29.

A8. Colombani, O.; Ruppel, M.; Burkhardt, M.; Drechsler, M.; Schumacher, M.; Gradzielski, M.; Schweins, R.; Muller, A. H. E., Structure of Micelles of Poly(n-butyl acrylate)-block-poly(acrylic acid) Diblock Copolymers in Aqueous Solution. Macromolecules 2007, 40 (12), 4351-4362.

A9. Colombani, O.; Ruppel, M.; Schubert, F.; Zettl, H.; Pergushov, D. V.; Muller, A. H. E., Synthesis of Poly(n-butyl acrylate)-block-poly(acrylic acid) Diblock Copolymers by ATRP and Their Micellization in Water. Macromolecules 2007, 40 (12), 4338-4350.

A10. Xu, F.; Xu, J. W.; Luo, Y. L., Impact of hydrogenation on physicochemical and biomedical properties of pH-sensitive PMAA-b-HTPB-b-PMAA triblock copolymer drug carriers. J Biomater Appl 2016, 30 (10), 1473-84.

A11. Felber, A. E.; Dufresne, M.-H.; Leroux, J.-C., pH-sensitive vesicles, polymeric micelles, and nanospheres prepared with polycarboxylates. Advanced Drug Delivery Reviews 2012, 64 (11), 979-992.

A12. Lele, B. S.; Hoffman, A. S., Mucoadhesive drug carriers based on complexes of poly(acrylic acid) and PEGylated drugs having hydrolysable PEG-anhydride-drug linkages. J Control Release 2000, 69 (2), 237-48.

A13. Serra, L.; Domenech, J.; Peppas, N. A., Engineering design and molecular dynamics of mucoadhesive drug delivery systems as targeting agents. European Journal of Pharmaceutics and Biopharmaceutics 2009, 71 (3), 519-528.

A14. Aguiar, J.; Carpena, P.; Molina-Bolivar, J. A.; Carnero Ruiz, C., On the determination of the critical micelle concentration by the pyrene 1:3 ratio method. Journal of Colloid and Interface Science 2003, 258 (1), 116-122.

A15. Cai, R.; Li, R.; Qian, J.; Xie, A.; Nie, K., The morphology and fabrication of nanostructured micelle by a novel block copolymer with linear-dendritic structure. Materials Science and Engineering: C 2013, 33 (4), 2070-2077.

A16. Megías, C.; Pedroche, J.; Yust, M. d. M.; Alaiz, M.; Giron-Calle, J.; Millán, F.; Vioque, J., Stability of sunflower protein hydrolysates in simulated gastric and intestinal fluids and Caco-2 cell extracts. LWT - Food Science and Technology 2009, 42 (9), 1496-1500.

A17. Fu, T. J.; Abbott, U. R.; Hatzos, C., Digestibility of food allergens and nonallergenic proteins in simulated gastric fluid and simulated intestinal fluid-a comparative study. J Agric Food Chem 2002, 50 (24), 7154-60.

A18. Torii, T.; Kanemitsu, K.; Wada, T.; Itoh, S.; Kinugawa, K.; Hagiwara, A., Measurement of short-chain fatty acids in human faeces using high-performance liquid chromatography: specimen stability. Ann Clin Biochem 2010, 47 (Pt 5), 447-52.

A19. Tyagi, A. M.; Yu, M.; Darby, T. M.; Vaccaro, C.; Li, J. Y.; Owens, J. A.; Hsu, E.; Adams, J.; Weitzmann, M. N.; Jones, R. M.; Pacifici, R., The Microbial Metabolite Butyrate Stimulates Bone Formation via T Regulatory Cell-Mediated Regulation of WNT10B Expression. Immunity 2018, 49 (6), 1116-1131.e7.

A20. Jiang, H.; Wang, Q.; Sun, X., Lymph node targeting strategies to improve vaccination efficacy. Journal of Controlled Release 2017, 267, 47-56.

A21. Wiig, H.; Swartz, M. A., Interstitial Fluid and Lymph Formation and Transport: Physiological Regulation and Roles in Inflammation and Cancer. Physiological Reviews 2012, 92 (3), 1005-1060.

A22. Ohnmacht, C.; Park, J.-H.; Cording, S.; Wing, J. B.; Atarashi, K.; Obata, Y.; Gaboriau-Routhiau, V.; Marques, R.; Dulauroy, S.; Fedoseeva, M.; Busslinger, M.; Cerf-Bensussan, N.; Boneca, I. G.; Voehringer, D.; Hase, K.; Honda, K.; Sakaguchi, S.; Eberl, G., The microbiota regulates type 2 immunity through RORyt+ T cells. Science 2015, 349 (6251), 989.

B1. Iweala, O.I. & Nagler, C.R. The Microbiome and Food Allergy. Annual Review of Immunology 37, 377-403 (2019).

B2. Bao, R., et al. Fecal microbiome and metabolome differ in healthy and food-allergic twins. J Clin Invest 131(2021).

B3. Feehley, T., et al. Healthy infants harbor intestinal bacteria that protect against food allergy. Nature Medicine (2019).

B4. Kim, C.H. Control of lymphocyte functions by gut microbiota-derived short-chain fatty acids. Cell Mol Immunol 18, 1161-1171 (2021).

B5. Tan, J., et al. The role of short-chain fatty acids in health and disease. Adv Immunol 121, 91-119 (2014).

B6. Zheng, L., et al. Microbial-Derived Butyrate Promotes Epithelial Barrier Function through IL-10 Receptor-Dependent Repression of Claudin-2. J Immunol 199, 2976-2984 (2017).

B7. Gupta, R.S., et al. Prevalence and Severity of Food Allergies Among US Adults. JAMA Netw Open 2, e185630-e185630 (2019).

B8. Chinthrajah, R.S., et al. Sustained outcomes in oral immunotherapy for peanut allergy (POISED study): a large, randomised, double-blind, placebo-controlled, phase 2 study. The Lancet 394, 1437-1449 (2019).

B9. Honda, K. & Littman, D.R. The microbiota in adaptive immune homeostasis and disease. Nature 535, 75-84 (2016).

B10. Belkaid, Y. & Harrison, O.J. Homeostatic Immunity and the Microbiota. Immunity 46, 562-576 (2017).

B11. Wells, J.M., et al. Homeostasis of the gut barrier and potential biomarkers. Am J Physiol Gastrointest Liver Physiol 312, G171-g193 (2017).

B12. Donohoe, D.R., et al. The microbiome and butyrate regulate energy metabolism and autophagy in the mammalian colon. Cell metabolism 13, 517-526 (2011).

B13. Koh, A., De Vadder, F., Kovatcheva-Datchary, P. & Bäckhed, F. From Dietary Fiber to Host Physiology: Short-Chain Fatty Acids as Key Bacterial Metabolites. Cell 165, 1332-1345 (2016).

B14. Kelly, C.J., et al. Crosstalk between Microbiota-Derived Short-Chain Fatty Acids and Intestinal Epithelial HIF Augments Tissue Barrier Function. Cell Host Microbe 17, 662- 671 (2015).

B15. Bevins, C.L. & Salzman, N.H. Paneth cells, antimicrobial peptides and maintenance of intestinal homeostasis. Nature Reviews Microbiology 9, 356-368 (2011).

B16. Arpaia, N., et al. Metabolites produced by commensal bacteria promote peripheral regulatory T-cell generation. Nature 504, 451-455 (2013).

B17. Furusawa, Y., et al. Commensal microbe-derived butyrate induces the differentiation of colonic regulatory T cells. Nature 504, 446 (2013).

B18. Smith, P.M., et al. The microbial metabolites, short-chain fatty acids, regulate colonic Treg cell homeostasis. Science 341, 569-573 (2013).

B19. Stefka, A.T., et al. Commensal bacteria protect against food allergen sensitization. Proc Natl Acad Sci U S A 111, 13145-13150 (2014).

B20. Nagler, C.R. Drugging the microbiome. J Exp Med 217, e20191642 (2020).

B21. Jimenez, M., Langer, R. & Traverso, G. Microbial therapeutics: New opportunities for drug delivery. J Exp Med 216, 1005-1009 (2019).

B22. Liu, H., et al. Butyrate: A Double-Edged Sword for Health? Adv Nutr 9, 21-29 (2018).

B23. Tan, J., et al. Dietary Fiber and Bacterial SCFA Enhance Oral Tolerance and Protect against Food Allergy through Diverse Cellular Pathways. Cell Reports 15, 2809-2824 (2016).

B24. Cait, A., et al. Microbiome-driven allergic lung inflammation is ameliorated by short-chain fatty acids. Mucosal Immunology 11, 785 (2017).

B25. Sun, M., et al. Microbiota-derived short-chain fatty acids promote Th1 cell IL-10 production to maintain intestinal homeostasis. Nature Communications 9, 3555 (2018).

B26. Xu, F., Xu, J.W. & Luo, Y.L. Impact of hydrogenation on physicochemical and biomedical properties of pH-sensitive PMAA-b-HTPB-b-PMAA triblock copolymer drug carriers. J Biomater Appl 30, 1473-1484 (2016).

B27. Colombani, O., et al. Synthesis of Poly(n-butyl acrylate)-block-poly(acrylic acid) Diblock Copolymers by ATRP and Their Micellization in Water. Macromolecules 40, 4338-4350 (2007).

B28. Colombani, O., et al. Structure of Micelles of Poly(n-butyl acrylate)-block-poly(acrylic acid) Diblock Copolymers in Aqueous Solution. Macromolecules 40, 4351-4362 (2007).

B29. Felber, A.E., Dufresne, M.-H. & Leroux, J.-C. pH-sensitive vesicles, polymeric micelles, and nanospheres prepared with polycarboxylates. Advanced Drug Delivery Reviews 64, 979-992 (2012).

B30. Aguiar, J., Carpena, P., Molina-Bolivar, J.A. & Camero Ruiz, C. On the determination of the critical micelle concentration by the pyrene 1:3 ratio method. Journal of Colloid and Interface Science 258, 116-122 (2003).

B31. Wu, H., Ting, J.M., Weiss, T.M. & Tirrell, M.V. Interparticle Interactions in Dilute Solutions of Polyelectrolyte Complex Micelles. ACS Macro Letters 8, 819-825 (2019).

B32. Torii, T., et al. Measurement of short-chain fatty acids in human faeces using high-performance liquid chromatography: specimen stability. Ann Clin Biochem 47, 447-452 (2010).

B33. Tyagi, A.M., et al. The Microbial Metabolite Butyrate Stimulates Bone Formation via T Regulatory Cell-Mediated Regulation of WNT10B Expression. Immunity 49, 1116-1131.e1117 (2018).

B34. Lele, B.S. & Hoffman, A.S. Mucoadhesive drug carriers based on complexes of poly(acrylic acid) and PEGylated drugs having hydrolysable PEG-anhydride-drug linkages. J Control Release 69, 237-248 (2000).

B35. Serra, L., Domenech, J. & Peppas, N.A. Engineering design and molecular dynamics of mucoadhesive drug delivery systems as targeting agents. European Journal of Pharmaceutics and Biopharmaceutics 71, 519-528 (2009).

B36. Tsuji, S., et al. Human intelectin is a novel soluble lectin that recognizes galactofuranose in carbohydrate chains of bacterial cell wall. J Biol Chem 276, 23456-23463 (2001).

B37. Castillo, P.A., et al. An Experimental Approach to Rigorously Assess Paneth Cell α-Defensin (Defa) mRNA Expression in C57BL/6 Mice. Scientific Reports 9, 13115 (2019).

B38. Cochran, K.E., Lamson, N.G. & Whitehead, K.A. Expanding the utility of the dextran sulfate sodium (DSS) mouse model to induce a clinically relevant loss of intestinal barrier function. PeerJ 8, e8681-e8681 (2020).

B39. Bashir, M.E.H., Louie, S., Shi, H.N. & Nagler-Anderson, C. Toll-Like Receptor 4 Signaling by Intestinal Microbes Influences Susceptibility to Food Allergy. The Journal of Immunology 172, 6978 (2004).

B40. Sorobetea, D., Holm, J.B., Henningsson, H., Kristiansen, K. & Svensson-Frej, M. Acute infection with the intestinal parasite Trichuris muris has long-term consequences on mucosal mast cell homeostasis and epithelial integrity. Eur J Immunol 47, 257-268 (2017).

B41. Bramhall, M. & Zaph, C. Mastering gut permeability: New roles for old friends. Eur J Immunol 47, 236-239 (2017).

B42. PALISADE Group of Clinical Investigators. AR101 oral immunotherapy for peanut allergy. New England Journal of Medicine 379, 1991-2001 (2018).

B43. Atarashi, K., et al. Induction of colonic regulatory T cells by indigenous Clostridium species. Science 331, 337-341 (2011).

B44. Lopetuso, L.R., Scaldaferri, F., Petito, V. & Gasbarrini, A. Commensal Clostridia: leading players in the maintenance of gut homeostasis. Gut Pathogens 5, 23 (2013).

B45. Byndloss, M.X., et al. Microbiota-activated PPAR-γ signaling inhibits dysbiotic Enterobacteriaceae expansion. Science 357, 570-575 (2017).

B46. Gupta, R.S., et al. The Public Health Impact of Parent-Reported Childhood Food Allergies in the United States. Pediatrics 142, e20181235 (2018).

B47. Rossen, N.G., et al. Findings From a Randomized Controlled Trial of Fecal Transplantation for Patients With Ulcerative Colitis. Gastroenterology 149, 110-118.e114 (2015).

B48. Nair, A.B. & Jacob, S. A simple practice guide for dose conversion between animals and human. J Basic Clin Pharm 7, 27-31 (2016).

B49. Vernero, M., et al. The Usefulness of Microencapsulated Sodium Butyrate Add-On Therapy in Maintaining Remission in Patients with Ulcerative Colitis: A Prospective Observational Study. Journal of clinical medicine 9, 3941 (2020).

B50. Luceri, C., et al. Effect of butyrate enemas on gene expression profiles and endoscopic/histopathological scores of diverted colorectal mucosa: A randomized trial. Digestive and Liver Disease 48, 27-33 (2016).

B51. Facchin, S., et al. Microbiota changes induced by microencapsulated sodium butyrate in patients with inflammatory bowel disease. Neurogastroenterology & Motility 32, e13914 (2020).

B52. Cai, R., Li, R., Qian, J., Xie, A. & Nie, K. The morphology and fabrication of nanostructured micelle by a novel block copolymer with linear-dendritic structure. Materials Science and Engineering: C 33, 2070-2077 (2013).

B53. Megías, C., et al. Stability of sunflower protein hydrolysates in simulated gastric and intestinal fluids and Caco-2 cell extracts. LWT - Food Science and Technology 42, 1496-1500 (2009).

B54. Fu, T.J., Abbott, U.R. & Hatzos, C. Digestibility of food allergens and nonallergenic proteins in simulated gastric fluid and simulated intestinal fluid-a comparative study. J Agric Food Chem 50, 7154-7160 (2002).

B55. Law, C.W., Chen, Y., Shi, W. & Smyth, G.K. voom: precision weights unlock linear model analysis tools for RNA-seq read counts. Genome Biology 15, R29 (2014).

B56. Callahan, B.J., et al. DADA2: High-resolution sample inference from Illumina amplicon data. Nature Methods 13, 581-583 (2016).

B57. Wang, Q., Garrity George, M., Tiedje James, M. & Cole James, R. Naive Bayesian Classifier for Rapid Assignment of rRNA Sequences into the New Bacterial Taxonomy. Applied and Environmental Microbiology 73, 5261-5267 (2007).

B58. McMurdie, P.J. & Holmes, S. phyloseq: An R Package for Reproducible Interactive Analysis and Graphics of Microbiome Census Data. PLOS ONE 8, e61217 (2013).

B59. Segata, N., et al. Metagenomic biomarker discovery and explanation. Genome Biology 12, R60 (2011).

B60. Yang, C. Microbiome R package: microbiome biomarker analysis toolkit. R package version 0.99.0. (2020).

B61. Turner, S., Pryer, K.M., Miao, V.P.W. & Palmer, J.D. Investigating Deep Phylogenetic Relationships among Cyanobacteria and Plastids by Small Subunit rRNA Sequence Analysis1. Journal of Eukaryotic Microbiology 46, 327-338 (1999).

B62. Amann, R.I., Ludwig, W. & Schleifer, K.H. Phylogenetic identification and in situ detection of individual microbial cells without cultivation. Microbiological Reviews 59, 143-169 (1995).

B63. Matsuki, T., et al. Development of 16S rRNA-gene-targeted group-specific primers for the detection and identification of predominant bacteria in human feces. Applied and environmental microbiology 68, 5445-5451 (2002).

What is claimed is:

1. A composition comprising a micelle of a copolymer of methacrylic acid (MAA) and N-(2-alkanoyloxyethyl) methacrylamide (AMA).

2. The composition of claim 1, wherein the copolymer is a block copolymer having the structure: 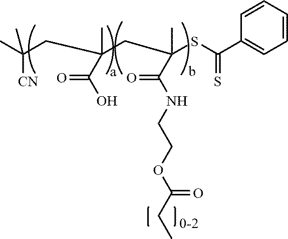
; wherein a and b are independently 1-1000.

3. The composition of claim 1, wherein the copolymer is a random copolymer having the structure: 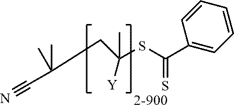
; wherein each Y is independently selected from: 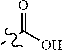
, and 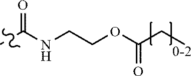
.

4. The composition of claim 1, wherein the copolymer is a block copolymer having the structure: 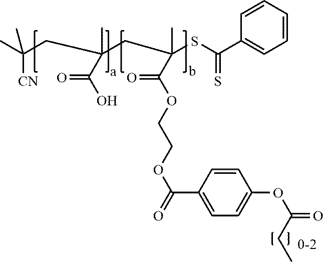
; wherein a and b are independently 1-1000.

5. The composition of claim 1, wherein the copolymer is a random copolymer having the structure: 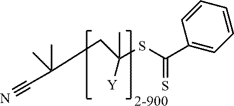
; wherein each Y is independently selected from: 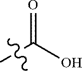
, and 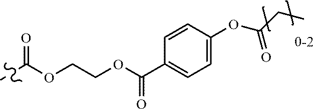
.

6. The composition of claim 1, wherein the copolymer is a block copolymer having the structure: 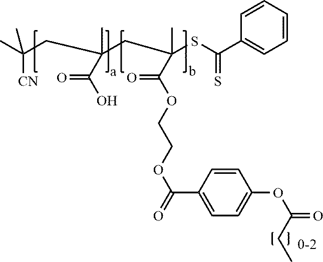
; wherein a and b are independently 1-1000.

7. The composition of claim 1, wherein the copolymer is a random copolymer having the structure: 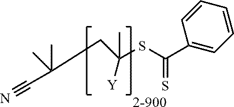
; wherein each Y is independently selected from: 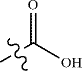
, and 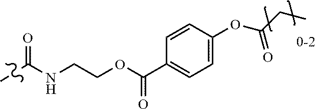
.

8. The composition of claim 1, wherein the copolymer is a block copolymer and has the structure: 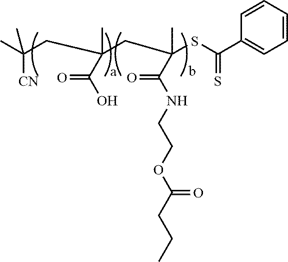
; wherein a and b are independently 1-1000.

9. The composition of claim 1, wherein the copolymer is a random copolymer and has the structure 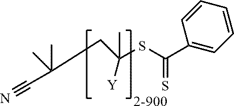
; wherein each Y is independently selected from: 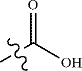
, and 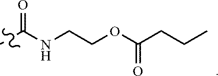
.

10. The composition of claim 1, wherein the copolymer is a block copolymer having the structure: 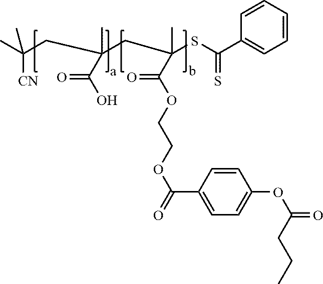
; wherein a and b are independently 1-1000.

11. The composition of claim 1, wherein the copolymer is a random copolymer and has the structure: 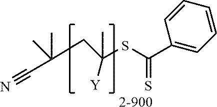
; wherein each Y is independently selected from: 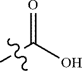
, and 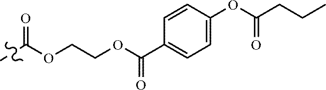
.

12. The composition of claim 1, wherein the copolymer is a block copolymer having the structure: 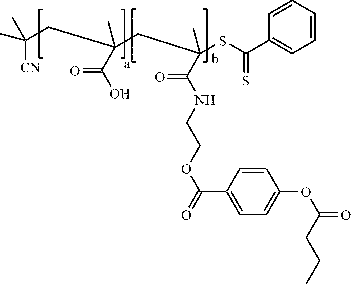
; wherein a and b are independently 1-1000.

13. The composition of claim 1, wherein the copolymer is a random copolymer having the structure: 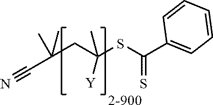
; wherein each Y is independently selected from: 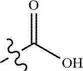
, and 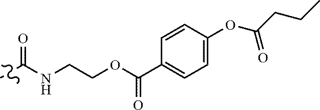
.

14. The composition of claim 1, further comprising a second micelle of a second copolymer of 2-hydroxypropyl methacrylamide (HPMA) and N-(2-alkanoyloxyethyl) methacrylamide (AMA).

15. The composition of claim 14, wherein the second copolymer is a block copolymer having the structure: 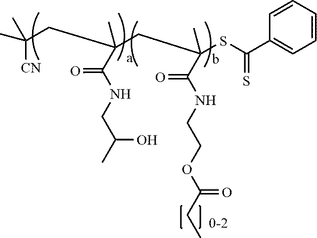
; wherein a and b are independently 1-1000.

16. The composition of claim 14, wherein the second copolymer is a random copolymer having the structure: 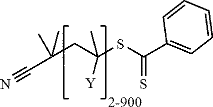
; wherein each Y is independently selected from: 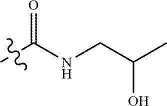
, and 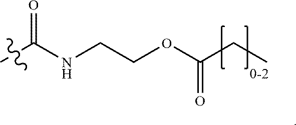
.

17. The composition of claim 14, wherein the second copolymer is a block copolymer having the structure: 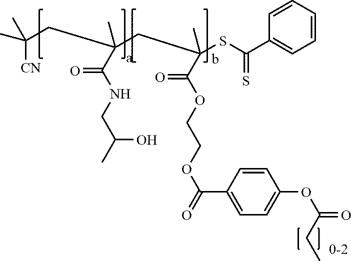
; wherein a and b are independently 1-1000.

18. The composition of claim 14, wherein the second copolymer is a random copolymer having the structure: 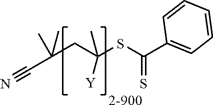
; wherein each Y is independently selected from: 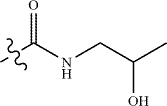
, and 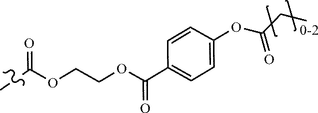
.

19. The composition of claim 14, wherein the second copolymer is a block copolymer having the structure: 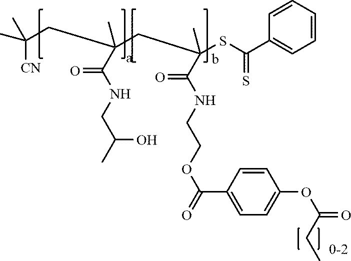
; wherein a and b are independently 1-1000.

20. The composition of claim 14, wherein the second copolymer is a random copolymer having the structure: 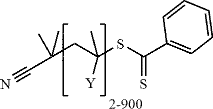
; wherein each Y is independently selected from: 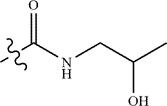
, and 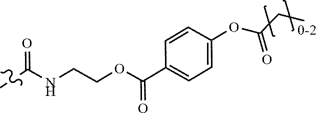
.

21. The composition of claim 14, wherein the second copolymer is a block copolymer and has the structure: 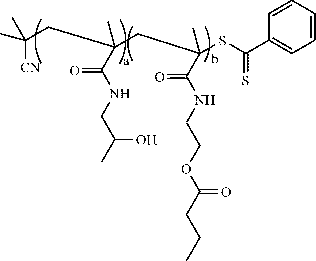
; wherein a and b are independently 1-1000.

22. The composition of claim 14, wherein the second copolymer is a random copolymer and has the structure 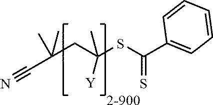
; wherein each Y is independently selected from: 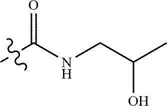
, and 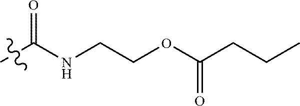
.

23. The composition of claim 14, wherein the second copolymer is a block copolymer having the structure: 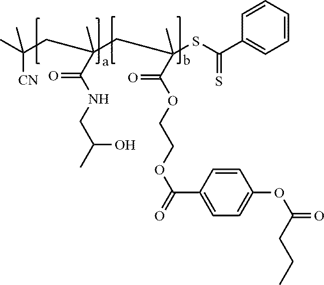
; wherein a and b are independently 1-1000.

24. The composition of claim 14, wherein the second copolymer is a random copolymer and has the structure: 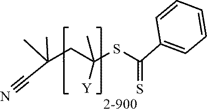
; wherein each Y is independently selected from: 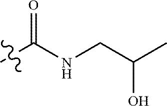
, and 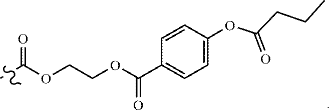
.

25. The composition of claim 14, wherein the second copolymer is a block copolymer having the structure: 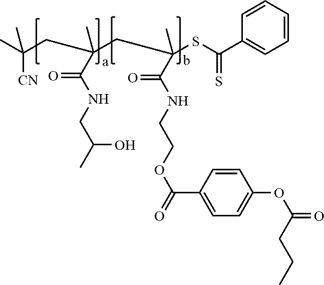
; wherein a and b are independently 1-1000.

26. The composition of claim 14, wherein the second copolymer is a random copolymer having the structure: 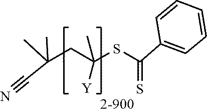
; wherein each Y is independently selected from: 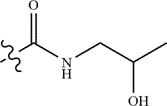
, and 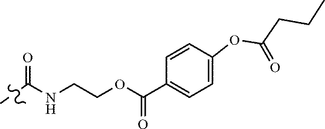
.
